(19) United States
(12) Patent Application Publication
(10) Pub. No.: US 2012/0220590 A1
(43) Pub. Date: Aug. 30, 2012

(54) NOVEL COMPOUNDS AS MODULATORS OF GLUCOCORTICOID RECEPTORS

(76) Inventors: Pravin S. Thombare, Gujarat (IN); Amitgiri Goswami, Gujarat (IN); Edouard Zamaratski, Huddinge (SE); Tomas Hansson, Huddinge (SE); Thomas Norin, Huddinge (SE)

(21) Appl. No.: 13/387,844

(22) PCT Filed: Jul. 30, 2010
(86) PCT No.: PCT/IN2010/000502

§ 371 Date: Mar. 14, 2012

(30) Foreign Application Priority Data
Jul. 31, 2009 (IN) 1769/MUM/2009

(51) Int. Cl. A61K 31/5377; A61K 31/34; C07D 333/20; A61K 31/381; C07D 213/57; A61K 31/44; C07D 401/12; A61K 31/4439; C07D 309/10; A61K 31/351; C07D 277/28; A61K 31/426; C07D 413/12; A61K 31/444; A61P 29/00; A61P 37/00; A61P 37/08; C07D 307/06

(52) U.S. Cl. 514/235.5; 549/491; 514/471; 549/75; 514/438; 546/330; 514/357; 546/279.1; 514/343; 549/416; 514/459; 548/205; 514/365; 544/131; 546/265; 514/332

(57) ABSTRACT

Compounds of formula (I) and pharmaceutical compositions containing them are described. The use of the compounds and compositions are also described.

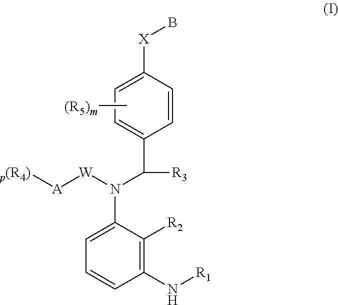

### FIELD OF INVENTION

The present invention relates to novel non-steroidal glucocorticoid ligands, their pharmaceutically acceptable salts, their pharmaceutically acceptable solvates and pharmaceutically acceptable compositions containing them and their use in modulating the glucocorticoid receptor function, treating disease-states or conditions mediated by physiological processes that can be modulated by the glucocorticoid receptor function in a patient in need of such treatment. Specifically are disclosed herein, a class of compounds of formula (I) which are dissociated glucocorticoid ligands and therefore are devoid or have minimum side effects which restrict the use of classical known glucocorticoid modulators. More particularly, the present invention relates to novel compounds of the general formula (I), their derivatives, their analogs, their tautomeric forms, their stereoisomers, their pharmaceutically acceptable salts, their pharmaceutically acceptable solvates, pharmaceutical compositions containing them, use of these compounds in medicine and the intermediates involved in their preparation.

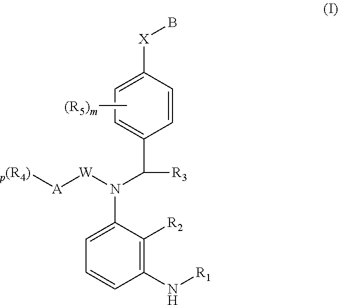

The present invention also relates to a process for the preparation of the compounds of formula (I), their derivatives, their analogs, their tautomeric forms, their stereoisomers, their pharmaceutically acceptable salts, their pharmaceutically acceptable solvates, and pharmaceutical compositions containing them.

The compounds of the general formula (I) suppress a variety of immune and inflammatory functions by inhibition of inflammatory cytokines such as IL-1, IL-6, and TNF-α, through glucocorticoid receptor mediated pathways and hence are useful in combating different medical conditions, where such inhibition of pro-inflammatory cytokines is beneficial. Thus, it could be used in the treatment of inflammatory, immune, and allergic disorders including rheumatic diseases such as rheumatoid arthritis, juvenile arthritis, Sjögren's syndrome, and ankylosing spondylitis, dermatological diseases including psoriasis and pemphigus, allergic disorders including allergic rhinitis, atopic dermatitis, and contact dermatitis, pulmonary conditions including asthma and chronic obstructive pulmonary disease (COPD), and other immune and inflammatory diseases including Crohn disease, ulcerative colitis, systemic lupus erythematosus, autoimmune chronic active hepatitis, and many other related conditions, as well as in suppression of organ transplant rejection and in treatment of cancers, both as antiproliferative agents in glucocorticoid-sensitive cancers including leukemias, lymphomas and multiple myeloma and for relief of cancer-induced swelling in various cancers.

As the compounds of the invention modulate the glucocorticoid receptor function, they have very useful anti-inflammatory and anti-allergic, immune-suppressive, and anti-proliferative activity and they can be used in patients as drugs, particularly in the form of pharmaceutical compositions as set forth below, for the treatment of disease-states and conditions.

### BACKGROUND OF THE INVENTION

Glucocorticoids, a class of corticosteroids, are endogenous hormones with profound effects on the immune system and multiple organ systems. They suppress a variety of immune and inflammatory functions by inhibition of inflammatory cytokines such as IL-1, IL-2, IL-6, and TNF-α inhibition of arachidonic acid metabolites including prostaglandins and leukotrienes, depletion of T-lymphocytes and reduction of the expression of adhesion molecules on endothelial cells (P. J. Barnes, Clin. Sci., 1998, 94, pp. 557-572; P. J. Barnes et al., Trends Pharmacol. Sci., 1993, 14, pp. 436-441). In addition to these effects, glucocorticoids stimulate glucose production in the liver and catabolism of proteins, play a role in electrolyte and water balance, reduce calcium absorption, and inhibit osteoblast function.

The anti-inflammatory and immune suppressive activities of endogenous glucocorticoids have stimulated the development of synthetic glucocorticoid derivatives including dexamethasone, prednisone, and prednisolone (L. Parente, Glucocorticoids, N. J. Goulding and R. J. Flowers (eds.), Boston: Birkhauser, 2001, pp. 35-54). These have found wide use in the treatment of inflammatory, immune, and allergic disorders including rheumatic diseases such as rheumatoid arthritis, juvenile arthritis, and ankylosing spondylitis, dermatological diseases including psoriasis and pemphigus, allergic disorders including allergic rhinitis, atopic dermatitis, and contact dermatitis, pulmonary conditions including asthma and chronic obstructive pulmonary disease (COPD), and other immune and inflammatory diseases including Crohn disease, ulcerative colitis, systemic lupus erythematosus, autoimmune chronic active hepatitis, osteoarthritis, tendonitis, and bursitis (J. Toogood, Glucocorticoids, N. J. Goulding and R. J. Flowers (eds.), Boston: Birkhauser, 2001, pp. 161-174). They have also been used to help prevent and/or suppress rejection in organ transplantation and as treatment of various cancer forms.

Unfortunately, in addition to the desired therapeutic effects of glucocorticoids, their use is associated with a number of adverse side effects, some of which can be severe and life-threatening. These include alterations in fluid and electrolyte balance, edema, weight gain, hypertension, muscle weakness, development or aggravation of diabetes mellitus, and osteoporosis. Such effects are seen with most known steroidal glucocorticoids. Therefore, a non-steroidal compound that exhibited a reduced side effect profile while maintaining the potent anti-inflammatory effects would be particularly desirable, especially when treating a chronic disease.

The effects of glucocorticoids are mediated at the cellular level by the glucocorticoid receptor (R. H. Oakley and J. Cidlowski, Glucocorticoids, N. J. Goulding and R. J. Flowers (eds.), Boston: Birkhauser, 2001, pp. 55-80). The glucocorticoid receptor is a member of a class of structurally related intracellular receptors that when coupled with a ligand can function as a transcription factor that affects gene expression (R. M. Evans, Science, 1988, 240, pp. 889-895). Several other members of this family of steroid receptors are known such as mineralocorticoid, progesterone, estrogen, and androgen receptors.

One of the most frequent undesirable actions of a glucocorticoid therapy is the so-called “steroid diabetes” [Hatz, H. J., Glucocorticoide: Immunologische Grundlagen, Pharmakologie and Therapierichtlinien (Glucocorticoids: Immunological Bases, Pharmacology and Therapy Guidelines), Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). Thus, it has been proposed to develop compounds which are glucocorticoid modulators having desired anti-inflammatory effects, but which does not show the above undesirable side effects. A molecular mechanism which can explain the beneficial anti-inflammatory effects and the undesired side effects has been proposed (e.g., S. Heck et al., EMBO J, 1994, 17, pp. 4087-4095; H. M. Reichardt et al., Cell, 1998, 93, pp. 531-541 etc.). Many of the metabolic and cardiovascular side effects are thought to be the result of a process called transactivation. In transactivation, the translocation of the ligand-bound glucocorticoid receptor to the nucleus is followed by binding to glucocorticoid response elements (GREs) in the promoter region of side effect-associated genes, for example, phosphoenolpyruvate carboxy kinase (PEPCK) in the case of increased glucose production. The result is an increased transcription rate of these genes, which is believed to result, ultimately, in the observed side effects. The anti-inflammatory effects are thought to be predominantly due to a process called transrepression. In general, transrepression is a process independent of DNA binding that results from inhibition by the ligand-bound glucocorticoid receptor of NF-κB and AP-1-mediated pathways, leading to down regulation of many inflammatory and immune mediators.

Additionally, it is believed that a number of the observed side effects may be due to the cross-reactivity of the currently available glucocorticoids with other steroid receptors, particularly the mineralocorticoid and progesterone receptors.

Thus, several ligands for the glucocorticoid receptor have been proposed that claim to be highly selective and show dissociation in the transactivation and transrepression pathways, providing therapeutic agents with a reduced side effect profile.

Novel non-steroidal ligands for the glucocorticoid receptor have been described in the scientific and patent literature. Several of them also claim to disclose compounds which show dissociation between anti-inflammatory and metabolic actions and thereby are expected to show the desired beneficial profile of dissociated glucocorticoid ligands. Such compounds are disclosed in for example, WO 99/33786, WO 99/41256; WO 99/63976, WO 00/66522, WO 00/032584, WO 02/10143, WO 02/010143, WO 03/082827, WO 2005/034939, WO2006/100100, WO2006/108712, WO2006/108699, WO 2006/046916, WO2007/000334, WO 2007/123465, WO 2007/114763, WO 2008/98798, WO 2008/075005, U.S. Pat. No. 5,688,810, U.S. Pat. No. 6,903,215, U.S. Pat. No. 6,323,199, DE 100 38 639.

WO 02/064550, discloses a large number of compounds as glucocorticoid receptor modulators. It discloses that several of the compounds disclosed therein show good binding to glucocorticoid receptors in vitro. However, no data is provided for the inflammatory potential of these compounds nor does it state whether the compounds disclosed show any favourable dissociation profile which is essential in order to overcome the side effects associated with known glucocorticoid compounds.

Given the efficacy demonstrated by available glucocorticoid drugs in inflammatory and immune diseases and their adverse side effects, there remains a need for novel glucocorticoid receptor agonists with selectivity over other members of the steroid receptor family and dissociation between the transactivation and transrepression activities. Though several compounds having such beneficial properties have been proposed in the literature, however, none of these compounds are yet to reach the market and so therefore there remains a need to develop newer medicines, which have the desired binding to the glucocorticoid receptor and exhibits the desired anti-inflammatory activities but have reduced or negligible side effects. We herein disclose such novel compounds.

### SUMMARY OF INVENTION

The objective of this invention is to develop novel compounds, which selectively bind to the glucocorticoid receptor and show anti-inflammatory activities but have reduced or negligible side effects. Thus, the present invention discloses novel compounds which show dissociation in the transactivation and transrepression pathways, and therefore are expected to show a reduced side effect profile.

The invention further provides a method of treating a disease characterized by inflammatory, allergic, or proliferative processes, in a patient in need of such treatment, the method comprising administering to the patient an effective amount of a pharmaceutically acceptable compound of formula (I), according to the invention or a prodrug, solvate, or salt thereof.

### OBJECTS OF THE INVENTION

An important object of the present invention is to provide novel compounds represented by the general formula (I), their pharmaceutically acceptable salts, their pharmaceutically acceptable solvates, and pharmaceutical compositions containing them or mixtures thereof which selectively bind to the glucocorticoid receptor and also shows anti-inflammatory activities but have reduced or negligible side effects.

In one embodiment, the present invention provides novel compounds represented by the general formula (I), their pharmaceutically acceptable salts, their pharmaceutically acceptable solvates, and pharmaceutical compositions containing them or their mixtures thereof having enhanced activities, without side effects or with reduced side effects.

In another embodiment, the present invention provides a process for the preparation of novel compounds represented by the general formula (I), their stereoisomers, their pharmaceutically acceptable salts, their pharmaceutically acceptable solvates.

In a still further embodiment, the present invention provides pharmaceutical compositions containing compounds of the general formula (I), their pharmaceutically acceptable salts, their pharmaceutically acceptable solvates or their mixtures in combination with suitable carriers, solvents, diluents and other media normally employed in preparing such compositions.

### DETAILED DESCRIPTION OF THE INVENTION

Accordingly, in one aspect of the invention there is provided a compound of the general formula (I)

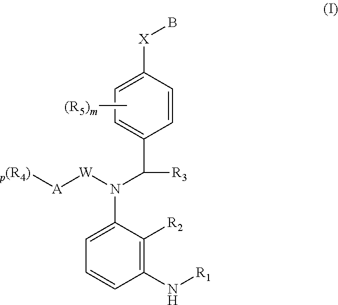

wherein, R₁ represents hydrogen or optionally substituted groups selected from linear or branched —(CH₂)rSO₂R₇, —(CH₂)r—C(O)R₇, —(CH₂)r—C(O)OR₇, —(CH₂)r—C(O)NR₇R₈, —(CH₂)r-C(O)NHOR₇, (CH₂)rSO₂—(CH₂)q-C(O)OR₇, (CH₂)rSO₂—(CH₂)q-OR₇ and SO₂—(CH₂)r—C(O)—NR₈; r and q independently represent an integer from 0-5;
R₇ and R₈ are the same or different and independently represent hydrogen, hydroxy, or optionally substituted groups selected from (C₁-C₆)alkyl, (C₂-C₄)alkenyl, and (C₂-C₄)alkynyl;
R₂ represents hydrogen, halogen, cyano, (CH₂)m-OR₇, or optionally substituted (C₁-C₆)alkyl; R₃ represents hydrogen, cyano or optionally substituted groups selected from (C₁-C₆)alkyl and (CH₂)m-O—R₇;
W represents —(CR₇R₈)k-; k represents an integer from 0-4; A represents hydrogen, optionally substituted (C₁-C₄)alkyl, phenyl or a heteroaromatic ring; R₄ at each occurrence independently represents halogen, (C₁-C₄)alkylene-hydroxy, nitro, cyano, CONH₂, —(C₁-C₆)alkyl-OC(O)R₇ or optionally substituted groups selected from (C₁-C₆)alkyl, (C₁-C₆)alkoxy and amino;
p represents an integer from 0-5;
R₅ at each occurrence is independently selected from hydrogen, halogen, hydroxy, cyano and optionally substituted groups selected from linear or branched (C₁-C₆)alkyl and (C₁-C₆)alkoxy;
m represents an integer from 0-4;
X represents —CH₂—, O or S;
B represents a cyclic group selected from phenyl and a heteroaromatic ring, wherein said cyclic group is substituted with a group La, and said cyclic group being optionally further substituted with one or more groups independently selected from R₆ and Lb;
La represents (CH₂)q′-Z, (CH₂)r′-O—(CH₂)q′-Z or CH═CH—Z;
r′ and q′ independently represents an integer from 0-4;
Z represents optionally substituted groups selected from (C₁-C₆)alkyl, (C₂-C₆)alkenyl, (C₂-C₆)alkynyl, (C₃-C₇)cycloalkyl, (C₁-C₆)alkoxy, —OSO₂R₇, —S(C₁-C₆)alkyl, phenyl, or a saturated heterocyclic group or a heteroaromatic ring, said saturated heterocyclic group or heteroaromatic ring optionally being substituted with one or more halogen, cyano or (C₁-C₆)alkyl;
R₆ at each occurrence independently represents halogen, hydroxy, cyano, —S(C₁-C₄)alkyl, or optionally substituted groups selected from linear or branched (C₁-C₆)alkyl and (C₁-C₆)alkoxy;
Lb is selected from T-(CH₂)r-D, wherein T is optionally present and independently represents O, S, —CH₂—, —NH—, —NHCO—, —CONH—, —SO₂NH—, or —NHSO₂—, and r represents an integer from 0-5;
D represents hydroxy, NHSO₂Rc, NHCORc, CONH₂, CONHRc, CONRdRe, SO₂NH₂, SO₃H, COOH, COORc, C(NH)NHOH, C(O)NHOH, C(O)NHORc, C(O)NHNH₂, NHC(O)NH₂ or a heteroaromatic ring;
Rc represents optionally substituted (C₁-C₃)alkyl or (C₃-C₇)cycloalkyl;
Rd and Re independently represent hydrogen, optionally substituted (C₁-C₃)alkyl, —O(C₁-C₃)alkyl or Rd and Re together form a —(CH₂)₄— group or a —(CH₂)₂—O—(CH₂)₂— group;
provided that when p is 0; then X is O and B is phenyl substituted with a group La, wherein La represents (CH₂)r′-O—(CH₂)q′-Z, r′ is 0 and q′ represents an integer from 0-3, and Z represents —S-methyl, an unsubstituted saturated heterocyclic group or a heteroaromatic ring;
or a pharmaceutically acceptable salt thereof.

In one embodiment of this aspect, there is provided a compound of formula (I), wherein R₁ represents —(CH₂)rSO₂R₇, —(CH₂)r—C(O)R₇, —(CH₂)r—C(O)OR₇, (CH₂)rSO₂—(CH₂)q—C(O)OR₇, (CH₂)qSO₂—(CH₂)q—OR₇ or SO₂—(CH₂)r—C(O)—NR₇R₈; and r and q independently represent 0, 1 or 2. More preferably, R₁ represents —(CH₂)rSO₂R₇, (CH₂)rSO₂—(CH₂)q—C(O)OR₇, (CH₂)qSO₂—(CH₂)q-OR₇ or SO₂—(CH₂)r-C(O)—NR₇R₈; and r and q independently represent 0, 1 or 2. Most preferably, R₁ represents —(CH₂)rSO₂R₇; and r represents 0, 1 or 2, especially r is 0.

In another embodiment of this aspect, there is provided a compound of formula (I), wherein R₇ and R₈ independently represent hydrogen, unsubstituted (C₁-C₆)alkyl or substituted (C₁-C₆)alkyl, especially halo-substituted (C₁-C₆)alkyl or cyano-substituted (C₁-C₆)alkyl. More preferably, R₇ and R₈ independently represent hydrogen, unsubstituted (C₁-C₃)alkyl or substituted (C₁-C₃)alkyl, especially halo-substituted (C₁-C₃)alkyl. Most preferably, R₇ and R₈ independently represent hydrogen, unsubstituted (C₁-C₃)alkyl or trifluoromethyl.

In another embodiment of this aspect, there is provided a compound of formula (I), wherein R₂ represents hydrogen or (C₁-C₆)alkyl, and R₃ represents hydrogen. More preferably, R₂ represents (C₁-C₃)alkyl, especially methyl.

In another embodiment of this aspect, there is provided a compound of formula (I), wherein W represents —(CR₇R₈)k; R₇ and R₈ represent hydrogen and k is 1.

In another embodiment of this aspect, there is provided a compound of formula (I), wherein A represents phenyl or pyridyl, especially phenyl.

In another embodiment of this aspect, there is provided a compound of formula (I), wherein each R₄ independently represents halogen, (C₁-C₄)alkylene-hydroxy, cyano, —(C₁-C₆)alkyl-OC(O)R₂, CONH₂, or optionally substituted (C₁-C₆)alkyl; and p is 0, 1 or 2, especially p is 1 or 2. In another embodiment of this aspect, there is provided a compound of formula (I), wherein R₄ is cyano and p is 1.

In another embodiment of this aspect, there is provided a compound of formula (I), wherein p is 0; X is O and B is selected from

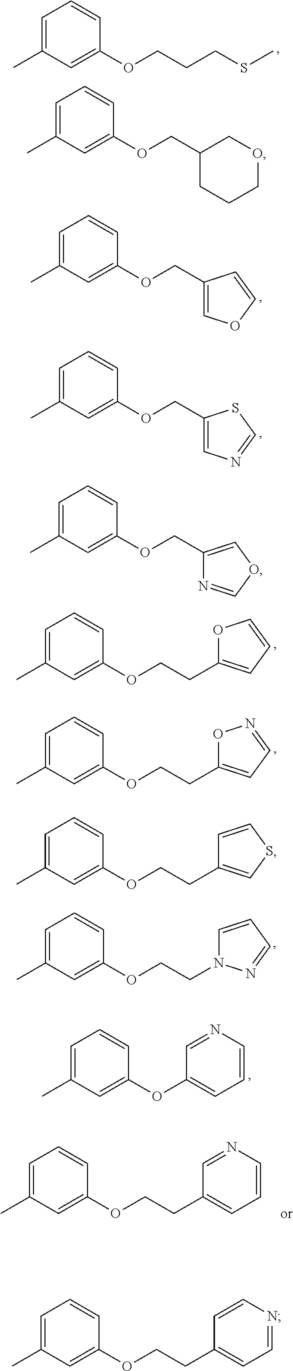

In a further embodiment of this aspect, there is provided a compound of formula (I), wherein p is 0; X is O and B is

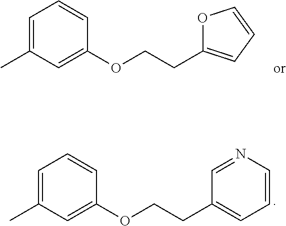

In another embodiment of this aspect, there is provided a compound of formula (I), wherein R₅ is hydrogen, halogen or a linear or branched (C₁-C₃)alkyl; and m is 1 or 2. In another embodiment of this aspect, there is provided a compound of formula (I), wherein m is 0.

In another embodiment of this aspect, there is provided a compound of formula (I), wherein X represents O; and B represents a cyclic group selected from phenyl and pyridyl, wherein said cyclic group is substituted with one La group. In a further embodiment of this aspect, there is provided a compound of formula (I), wherein X represents O; and B represents phenyl, substituted with one La group.

In another embodiment of this aspect, there is provided a compound of formula (I), wherein X represents O; and B represents a cyclic group selected from phenyl and pyridyl, wherein said cyclic group is substituted with one La group, and said cyclic group being further substituted with either one R₆ group or one Lb group.

In another embodiment of this aspect, there is provided a compound of formula (I), wherein La represents (CH₂)r′-O—(CH₂)q′-Z, (CH₂)q′-Z or CH═CH—Z; r′ and q′ independently represent an integer from 0-4; and Z represents (C₂-C₆)alkenyl, (C₁-C₆)alkoxy, —SO₂R₇, S(C₁-C₆)alkyl, phenyl optionally substituted with one hydroxy, a saturated heterocyclic group or a heteroaromatic ring, said saturated heterocyclic group or heteroaromatic ring optionally being substituted with one (C₁-C₆)alkyl. In this embodiment, Z more preferably represents —S(C₁-C₆)alkyl, phenyl optionally substituted with one hydroxy, or a saturated heterocyclic group or a heteroaromatic ring, said saturated heterocyclic group or heteroaromatic ring optionally being substituted with one (C₁-C₆)alkyl. Most preferably, Z represents a saturated heterocyclic group or a heteroaromatic ring, said saturated heterocyclic group or heteroaromatic ring optionally being substituted with one (C₁-C₄)alkyl. Most particularly preferably, Z represents an unsubstituted saturated heterocyclic group or an unsubstituted heteroaromatic ring, especially a heteroaromatic ring. Preferred heteroaromatic Z groups include pyridyl, thienyl, furyl, pyrrolyl, oxazolyl, thiazolyl, imidazolyl and isoxazolyl; particularly preferred heteroaromatic Z groups pyridyl, thienyl and furyl. For example, Z may represent pyridyl.

Preferably, La represents (CH₂)r′-O—(CH₂)q′-Z, wherein r′ is 0 and q′ represents 0, 1 or 2. More preferably, La represents (CH₂)r′-O—(CH₂)q′-Z, wherein r′ is 0 and q′ represents 1 or 2. Most preferably, La represents (CH₂)r′—O—(CH₂)q′-Z, wherein r′ is 0 and q′ is 1.

Particularly preferred La groups are the following:

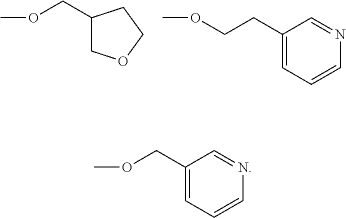

In another embodiment of this aspect, there is provided a compound of formula (I), wherein R₆ is halogen or an optionally substituted (C₁-C₄)alkyl, especially R₆ is halogen.

In another embodiment of this aspect, there is provided a compound of formula (I), wherein Lb is selected from T-(CH₂)r-D, wherein T is optionally present and independently represents —NHCO— or —CONH—, and wherein r represents an integer from 0-5, especially r is 0, 1 or 2. In a further embodiment of this aspect, T is present and represents —NHCO— or —CONH—, especially T is —CONH—.

In another embodiment of this aspect, there is provided a compound of formula (I), wherein D represents hydroxy, CONH₂, CONHRc, CONRdRe, COOH, COORc, C(O)NHORc, C(O)NHNH₂, or pyridyl. More preferably, D represents hydroxy, CONH₂, COOH, CONHRc, C(O)NHORc, C(O)NHNH₂, or pyridyl. Most preferably, D represents hydroxy, CONH₂, COOH, or CONHRc.

In another embodiment of this aspect, there is provided a compound of formula (I), wherein Rc represents optionally substituted (C₁-C₃)alkyl or cyclopropyl.

In another embodiment of this aspect, there is provided a compound of formula (I), wherein Rd and Re independently represent hydrogen, optionally substituted (C₁-C₃)alkyl, —O(C₁-C₃)alkyl or Rd and Re together form a —(CH₂)₄— group or a —(CH₂)₂—O—(CH₂)₂— group.

Accordingly, the present invention also provides a compound of formula (I), wherein

R₁ represents —(CH₂)rSO₂R₇;
r represents 0;
R₇ represents optionally substituted (C₁-C₃)alkyl;
R₂ represents hydrogen or methyl;
R₃ represents hydrogen;
W represents —(CR₇R₈)k, wherein R₇ and R₈ represent hydrogen and k is 1;
A represents phenyl or pyridyl;
R₄ independently represents halogen, (C₁-C₄)alkylene-hydroxy, cyano, CONH₂, —(C₁-C₆)alkyl-OC(O)R₇ or optionally substituted (C₁-C₆)alkyl;
p represents an integer from 0-2;
R₅ represents hydrogen, halogen;
m represents 0 or 1;
X represents O;
B represents a cyclic group selected from phenyl and pyridyl, wherein said cyclic group is substituted with one La group, and said cyclic group being optionally further substituted with one R₆ group or one Lb group; La represents (CH₂)q′-Z, (CH₂)r′-β-(CH₂)q′-Z or CH═CH—Z; r′ and q′ independently represent an integer from 0-4;
Z represents —S(C₁-C₆)alkyl, phenyl optionally substituted with one hydroxy, or a saturated heterocyclic group or a heteroaromatic ring, said saturated heterocyclic group or heteroaromatic ring optionally being substituted with one (C₁-C₆)alkyl;
R₆ represents halogen, cyano, —S(C₁-C₄)alkyl, or optionally substituted (C₁-C₄)alkyl;
Lb is selected from T-(CH₂)r-D, wherein T is optionally present and independently represents —NHCO— or —CONH—, and r represents 0, 1 or 2;
D represents hydroxy, CONH₂, CONHRc, CONRdRe, COOH, COORc, C(O)NHORc, C(O)NHNH₂, or pyridyl;
Rc represents optionally substituted (C₁-C₃)alkyl or cyclopropyl;
Rd and Re independently represent hydrogen, optionally substituted (C₁-C₃)alkyl, —O(C₁-C₃)alkyl or Rd and Re together form a —(CH₂)₄— group or a —(CH₂)₂—O—(CH₂)₂— group;
provided that when p is 0; then B is selected from

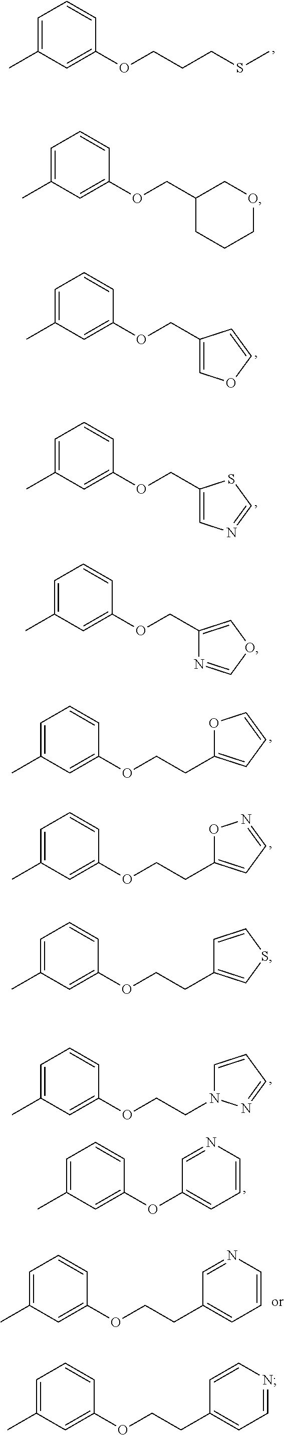

or a pharmaceutically acceptable salt thereof.
In another aspect, the present invention also provides a compound of formula (I), wherein
B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re independently represent hydrogen or together form a —(CH₂)₄— group or a —(CH₂)₂—O—(CH₂)₂— group, provided that when La is

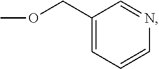

then the Lb group is not CONH₂;
phenyl or pyridyl substituted with one La group only, wherein the La group is selected from one of the following groups:

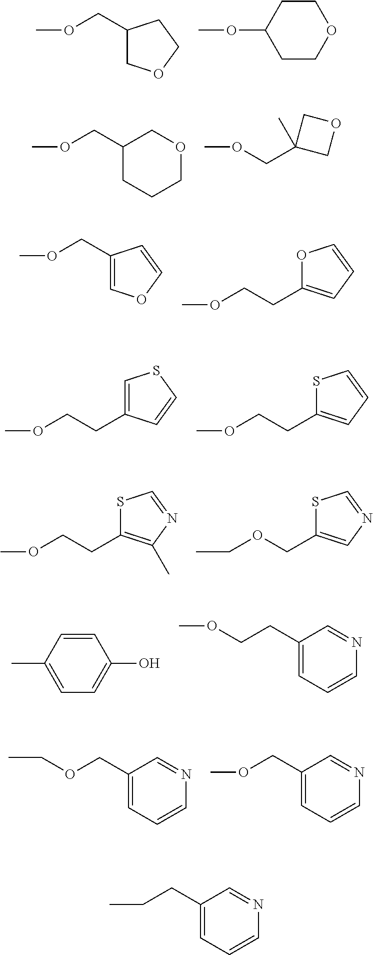

provided that
when La is

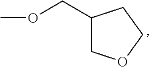

and R₇ is optionally substituted methyl, then either p represents 1 and R₄ is a methyl, trifluoromethyl or cyano group which is attached at the 4-position of the phenyl or pyridyl A ring relative to group W, or p represents 2 and one of the two R₄ groups is attached at the 4-position of the phenyl or pyridyl A ring relative to group W;
when La is

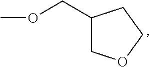

and R₇ is optionally substituted (C₂-C₃)alkyl then p represents 1 and R₄ is a cyano group which is attached at the 3-position of the phenyl or pyridyl A ring relative to group W;
when La is

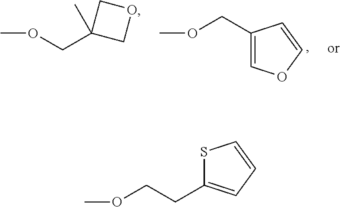

then p represents 1 and R₄ is a cyano group which is attached at the 4-position of the phenyl or pyridyl A ring relative to group W;
when La is

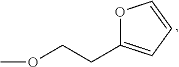

then either p represents 0, or p represents 1 and R₄ is a cyano group which is attached at the 4-position of the phenyl or pyridyl A ring relative to group W; and
when La is

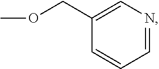

then either R₇ is methyl, p represents 1 and R₄ is a cyano group which is attached at the 4-position of the phenyl or pyridyl A ring relative to group W, or R₇ is ethyl, p represents 1 and R₄ is a cyano group which is attached at the 3-position of the phenyl or pyridyl A ring relative to group W.

In a further aspect, the present invention provides a compound of formula (I), wherein

R₂ represents methyl;
W represents —(CR₇R₈)k, wherein R₇ and R₈ represent hydrogen and k is 1;
B is selected from one of the following:
phenyl substituted with one La group, said phenyl being further substituted with one R₆ group or one Lb group, wherein the R₆ group represents halogen, —S(C₁-C₄)alkyl, or optionally substituted (C₁-C₄)alkyl, and wherein the Lb group is selected from T-(CH₂)r-D, wherein T is optionally present and represents —CONH—; D represents hydroxy, CONH₂, —COOH, CONHRc, C(O)NHORc, C(O)NHNH₂, or pyridyl, wherein Rc represents optionally substituted (C₁-C₃)alkyl or cyclopropyl, provided that
   when La is

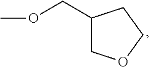

then the phenyl is further substituted one Lb group and the Lb group represents —(CH₂)r-D, wherein D represents CONH₂ or CONHRc;
phenyl substituted with one La group only, wherein the La group is selected from one of the following groups:

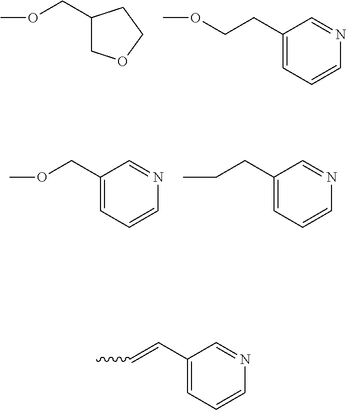

provided that
when La is

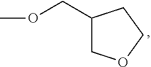

then R₇ is methyl, p represents 1, and R₄ is either a cyano group which is attached at the 3-position or the 4-position of the phenyl A ring relative to group W, or a methyl group which is attached at the 4-position of the phenyl A ring relative to group W;
when La is

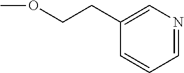

then R₇ represents unsubstituted (C₁-C₃)alkyl, and either p represents 0, or p represents 1 and R₄ is halogen, cyano or methyl; and
when La is

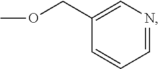

then either R₇ is methyl, p represents 1 and R₄ is a cyano; or R₇ is ethyl, p represents 1 and R₄ is a cyano group which is attached at, the 4-position of the phenyl or A ring relative to group W; or
pyridyl substituted with one La group only, wherein the La group represents (CH₂)₂—Z, wherein Z represents pyridyl.

In a still further aspect, the present invention provides a compound of formula (I), wherein

R₂ represents methyl; and
B is selected from one of the following:
phenyl substituted with one La group, said phenyl being further substituted with one R₆ group or one Lb group, wherein the R₆ group represents cyano, halogen, or —S(C₁-C₄)alkyl, and wherein the Lb group is T-(CH₂)r-D, in which either:
   T is absent, r represents 0 or 1, and D represents hydroxy, CONH₂, CONHRc, CONRdRe, C(O)NHNH₂, wherein Rc represents optionally substituted (C₁-C₃)alkyl or cyclopropyl, and Rd and Re independently represent hydrogen, optionally substituted (C₁-C₃)alkyl; or
   T is present and represents —CONH—, r represents 1 or 2, and D represents COOH or COORc, wherein Rc represents optionally substituted (C₁-C₃)alkyl; or
   T is present and represents —NHCO—, r represents 1 or 2, and D represents COOH or COORc, wherein Rc represents optionally substituted (C₁-C₃)alkyl; or;
phenyl substituted with one La group only, wherein the La group is selected from one of the following groups:

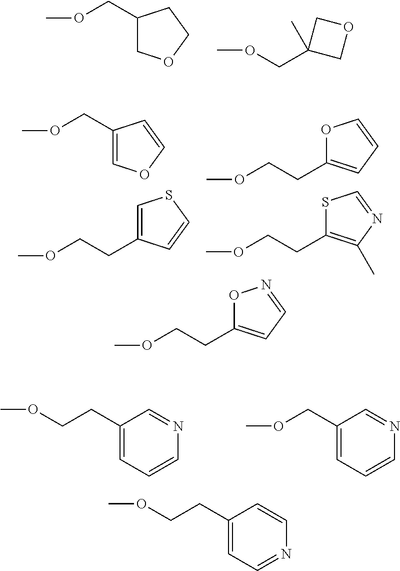

provided that
when La is

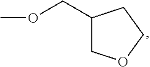

then either R₇ is ethyl, p is 0, 1 or 2, and R₄ is halogen, cyano, CONH₂, or optionally substituted (C₁-C₄)alkyl; or
   R₇ is methyl, p represents 1, and either R₄ is a methyl or halogen group which is attached at the 3-position of the phenyl A ring relative to group W, or R₄ is a
methyl, cyano or halogen group which is attached at the 4-position of the phenyl A ring relative to group W; or
   R₇ is methyl, p represents 2, R₄ is halogen, cyano, or methyl, and one of the two R₄ groups is attached at the 3-position of the phenyl A ring relative to group W
   when La is

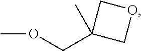

then R₄ is a halogen; and
when La is

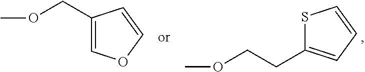

then R₇ is methyl, p represents 1, and R₄ is a cyano group which is attached at the 4-position of the phenyl A ring relative to group W; and
when La is

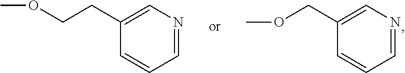

then R₇ represents unsubstituted (C₁-C₃)alkyl, and either p represents 0, or p represents 1 and R₄ is cyano, CONH₂, or methyl.

In an alternative embodiment, the present invention provides a compound of formula (I), wherein

B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re together form a —(CH₂)₄— group or a —(CH₂)₂—O—(CH₂)₂— group;
phenyl or pyridyl substituted with one La group only, wherein the La group is selected from one of the following groups:

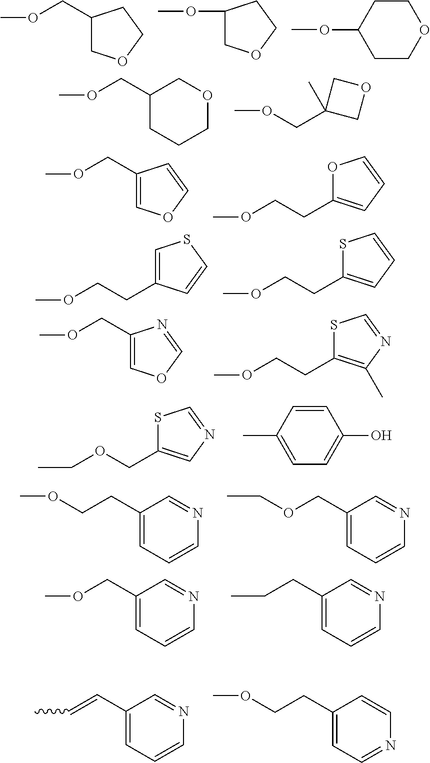

provided that
   when La is

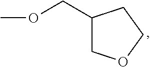

and R₇ is optionally substituted methyl,
then either p represents 1 and R₄ is selected from a cyano group which is attached at the 3-position or the 4-position of the phenyl or pyridyl A ring relative to group W, or a methyl or trifluoromethyl which is attached at the 4-position of the phenyl or pyridyl A ring relative to group W; or p represents 2 and one of the two R₄ groups is attached at the 4-position of the phenyl or pyridyl A ring relative to group W;
when La is

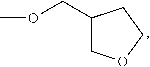

and R₇ is optionally substituted (C₂-C₃)alkyl,
then p represents 1 and R₄ is a cyano group which is attached at the 3-position of the phenyl or pyridyl A ring relative to group W;
when La is

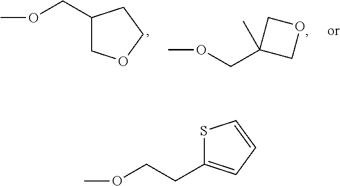

then p represents 1 and R₄ is a cyano group which is attached at the 4-position of the phenyl or pyridyl A ring relative to group W;
when La is

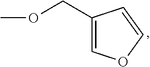

then either R₇ is methyl, p represents 1 and R₄ is a cyano group which is attached at the 4-position of the phenyl or pyridyl A ring relative to group W; or R₇ is ethyl, p represents 1 and R₄ is a cyano group;
when La is

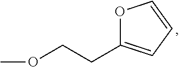

then either p represents 0, or p represents 1 and R₄ is a cyano group which is attached at the 4-position of the phenyl or pyridyl A ring relative to group W;
when La is

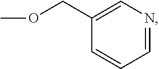

then R₇ represents unsubstituted (C₁-C₃)alkyl, and either p represents 0, or p represents 1 and R₄ is cyano, CONH₂, or methyl.

In a further aspect, the present invention provides a compound of formula (I), wherein

R₂ represents methyl;
B is selected from one of the following:
phenyl substituted with one La group, wherein the La group is:

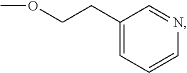

said phenyl being further substituted with one R₆ group or one Lb group, wherein the R₆ group represents cyano, halogen, or —S(C₁-C₄)alkyl, and wherein the Lb group is selected from T-(CH₂)r-D, in which either:
   T is absent, r represents 0 or 1, and D represents hydroxy, CONH₂, CONHRc, C(O)NHORc, or C(O)NHNH₂, wherein Rc represents optionally substituted (C₁-C₃)alkyl or cyclopropyl; or
   T is present and represents —CONH—, r represents 1 or 2, and D represents COORc or pyridyl, wherein Rc represents optionally substituted (C₁-C₃)alkyl; or
phenyl substituted with one La group, wherein the La group is

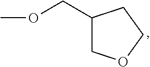

then the phenyl is further substituted one Lb group and the Lb group represents —(CH₂)r-D, wherein D represents CONH₂ or CONHRc
wherein Rc represents optionally substituted (C₁-C₃)alkyl; or
phenyl or pyridyl substituted with one La group only, wherein the La group is selected from one of the following groups:

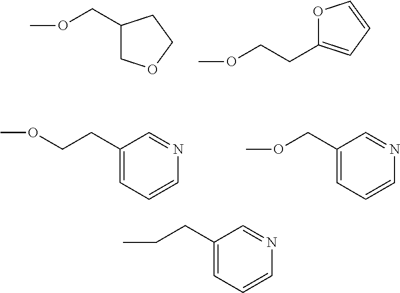

provided that
   when La is

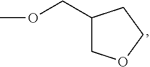

then R₇ is methyl, p represents 1, and R₄ is methyl;
when La is

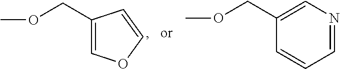

then R₇ is methyl, p represents 1, and R₄ is a cyano group which is attached at the 4-position of the phenyl A ring relative to group W; and
when La is

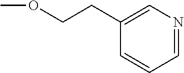

then R₇ represents unsubstituted (C₁-C₃)alkyl, and either p represents 0, or p represents 1 and R₄ is halogen, cyano or methyl.

In another embodiment, the present invention provides a compound of formula (I), wherein

B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re together form a —(CH₂)₄— group or a —(CH₂)₂—O—(CH₂)₂— group;
phenyl or pyridyl substituted with one La group only, wherein the La group is selected from one of the following groups:

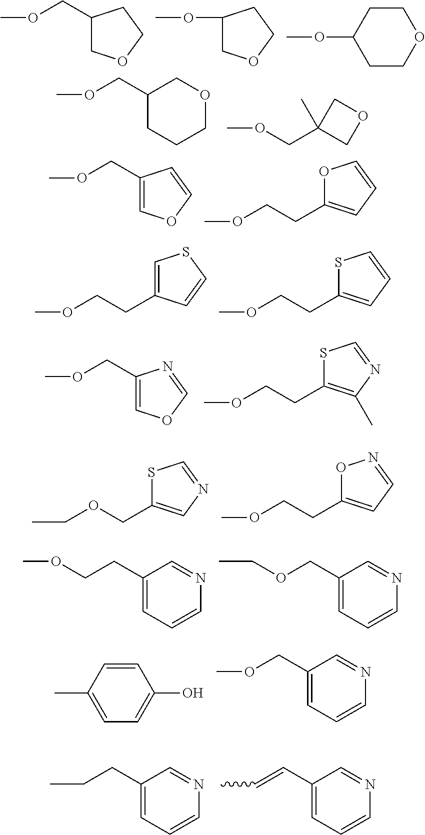

provided that
   when La is

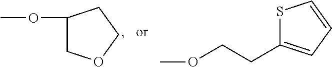

then p represents 1 and R₄ is a cyano group which is attached at the 4-position of the phenyl or pyridyl A ring relative to group W;
when La is

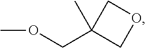

then p represents 1 and either R₄ is a halogen or R₄ is a cyano group which is attached at the 4-position of the phenyl or pyridyl A ring relative to group W;
when La is

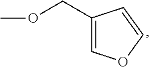

then either R₇ is methyl, p represents 1 and R₄ is a cyano group which is attached at the 4-position of the phenyl or pyridyl A ring relative to group W; or R₇ is ethyl, p represents 1 and R₄ is a cyano group; and
when La is

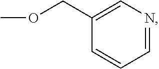

then R₇ represents unsubstituted (C₁-C₃)alkyl, and either p′ represents 0, or p represents 1 and R₄ is cyano, CONH₂, or methyl.

In another embodiment of this aspect, there is provided a compound of formula (I), said compound being selected from:
N-(3-((2,4-difluorobenzyl)(4-(3-((tetrahydrofuran-3-yl)methoxy)phenoxy)benzyl)amino)-2-methylphenyl)methanesulfonamide;
N-(3-((2-cyanobenzyl)(4-(3-(2-(thiophen-3-yl)ethoxy)phenoxy)benzyl)amino)-2-methylphenyl)methanesulfonamide;
N-(2-methyl-3-((4-(3-((tetrahydrofuran-3-yl)methoxy)phenoxy)benzyl)(4-(trifluoromethyl)benzyl)amino)phenyl)methanesulfonamide;
N-(3-((3-cyanobenzyl)(4-(3-(furan-3-ylmethoxy)phenoxy)benzyl)amino)-2-methylphenyl)ethanesulfonamide;
N-(3-((3-cyanobenzyl)(4-(3-(tetrahydrofuran-3-yloxy)phenoxy)benzyl)amino)-2-methylphenyl)methanesulfonamide;
N-(3-((3-cyanobenzyl)(4-(3-((3-methyloxetan-3-yl)methoxy)phenoxy)benzyl)amino)-2-methylphenyl)methanesulfonamide;
N-(3-((4-cyanobenzyl)(4-(3-((tetrahydrofuran-3-yl)oxy)phenoxy)benzyl)amino)-2-methylphenyl)ethanesulfonamide;
N-(3-((4-cyanobenzyl)(4-(3-((tetrahydrofuran-3-yl)oxy)phenoxy)benzyl)amino)-2-methylphenyl)methanesulfonamide;
N-(3-((3-cyanobenzyl)(4-(3-((tetrahydrofuran-3-yl)methoxy)phenoxy)benzyl)amino)-2-methylphenyl)propane-2-sulfonamide;
N-(3-((4-cyanobenzyl)(4-(3-(2-(pyridin-3-yl)ethoxy)phenoxy)benzyl)amino)-2-methylphenyl)-1,1,1-trifluoromethanesulfonamide;
N-(3-((3-cyanobenzyl)(4-(3-(2-(thiophen-3-yl)ethoxy)phenoxy)benzyl)amino)-2-methylphenyl)methanesulfonamide;
N-(3-(benzyl(4-(3-(2-(thiophen-3-yl)ethoxy)phenoxy)benzyl)amino)-2-methylphenyl)ethanesulfonamide;
N-(3-(benzyl(4-(3-(2-(thiophen-3-yl)ethoxy)phenoxy)benzyl)amino)-2-methylphenyl)methanesulfonamide hydrochloride;
N-(3-((4-cyanobenzyl)(4-(3-(2-(pyridin-3-yl)ethoxy)-5-(pyrrolidine-1-carbonyl)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-5-(2-(pyridin-3-yl)ethoxy)benzoic acid;
N-(3-((3-cyanobenzyl)(4-(3-((tetrahydrofuran-3-yl)methoxy)phenoxy)benzyl)amino)-2-methylphenyl)ethanesulfonamide;
N-(3-((3-cyanobenzyl)(4-(3-(2-(thiophen-2-yl)ethoxy)phenoxy)benzyl)amino)-2-methylphenyl)methanesulfonamide];
N-(3-((4-cyanobenzyl)(4-(3-(2-(pyridin-3-yl)ethoxy)phenoxy)benzyl)amino)phenyl)methanesulfonamide;
N-(3-((3-cyanobenzyl)(4-(3-((tetrahydrofuran-3-yl)methoxy)phenoxy)benzyl)amino)-2-methylphenyl)propane-1-sulfonamide;
N-(3-((4-cyanobenzyl)(4-(3-(furan-3-ylmethoxy)phenoxy)benzyl)amino)-2-methylphenyl)ethanesulfonamide;
3-(4-(((4-cyanobenzyl)(2-methyl-3-(methylsulfonamido)phenyl)amino)methyl)phenoxy)-N-(2-hydroxyethyl)-5-((tetrahydrofuran-3-yl)methoxy)benzamide;
N-(3-((4-cyanobenzyl)(4-(3-((tetrahydro-2H-pyran-4-yl)oxy)phenoxy)benzyl)amino)-2-methylphenyl)methanesulfonamide;
N-(3-((4-cyanobenzyl)(4-(3-(2-(4-methylthiazol-5-yl)ethoxy)phenoxy)benzyl)amino)-2-methylphenyl)methanesulfonamide;
N-(3-((4-cyanobenzyl)(4-(3-((3-methyloxetan-3-yl)methoxy)phenoxy)benzyl)amino)-2-methylphenyl)methanesulfonamide;
N-(3-((3-cyanobenzyl)(4-(3-(furan-3-ylmethoxy)phenoxy)benzyl)amino)-2-methylphenyl)methanesulfonamide;
N-(3-(benzyl(4-(3-(furan-3-ylmethoxy)phenoxy)benzyl)amino)-2-methylphenyl)ethanesulfonamide;
N-(3-((4-cyanobenzyl)(4-(3-(2-(thiophen-3-yl)ethoxy)phenoxy)benzyl)amino)-2-methylphenyl)methanesulfonamide;
N-(3-((3-fluorobenzyl)(4-(3-(furan-3-ylmethoxy)phenoxy)benzyl)amino)-2-methylphenyl)methanesulfonamide;
N-(3-((3,5-difluorobenzyl)(4-(3-((tetrahydrofuran-3-yl)methoxy)phenoxy)benzyl)amino)-2-methylphenyl)methanesulfonamide;
N-(3-((4-cyanobenzyl)(4-(3-(morpholine-4-carbonyl)-5-(2-(pyridin-3-yl)ethoxy)phenoxy)benzyl)amino)-2-methylphenyl)methanesulfonamide;
N-(3-((3-fluorobenzyl)(4-(3-((3-methyloxetan-3-yl)methoxy)phenoxy)benzyl)amino)-2-methylphenyl)methanesulfonamide;
N-(3-((4-cyanobenzyl)(4-(3-((tetrahydrofuran-3-yl)methoxy)phenoxy)benzyl)amino)-2-methylphenyl)ethanesulfonamide;
N-(3-((4-cyanobenzyl)(4-(3-(furan-3-ylmethoxy)phenoxy)benzyl)amino)-2-methylphenyl)methanesulfonamide;
N-(3-((3-fluorobenzyl)(4-(3-((tetrahydrofuran-3-yl)methoxy)phenoxy)benzyl)amino)-2-methylphenyl)ethanesulfonamide;
N-(3-((4-cyanobenzyl)(4-(3-(2-(thiophen-2-yl)ethoxy)phenoxy)benzyl)amino)-2-methylphenyl)methanesulfonamide;
2-(3-(4-(((4-cyanobenzyl)(2-methyl-3-(methylsulfonamido)phenyl)amino)methyl)phenoxy)-5-(2-(pyridin-3-yl)ethoxy)benzamido)acetic acid;
N-(3-((4-chloro-3-fluorobenzyl)(4-(3-((tetrahydrofuran-3-yl)methoxy)phenoxy)benzyl)amino)-2-methylphenyl)methanesulfonamide;
4-(((2-methyl-3-(methylsulfonamido)phenyl)(4-(3-(2-(pyridin-3-yl)ethoxy)phenoxy)benzyl)amino)methyl)benzamide;
methyl 3-(4-(((4-cyanobenzyl)(2-methyl-3-(methylsulfonamido)phenyl)amino)methyl)phenoxy)-5-(2-(pyridin-3-yl)ethoxy)benzoate;
3-(4-(((4-cyanobenzyl)(2-methyl-3-(methylsulfonamido)phenyl)amino)methyl)phenoxy)-N-methyl-5-(pyridin-3-ylmethoxy)benzamide;
N-(2-methyl-3-((3-methylbenzyl)(4-(3-((tetrahydrofuran-3-yl)methoxy)phenoxy)benzyl)amino)phenyl)methanesulfonamide;
ethyl 2-(3-(4-(((4-cyanobenzyl)(2-methyl-3-(methylsulfonamido)phenyl)amino)methyl)phenoxy)-5-((tetrahydrofuran-3-yl)methoxy)benzamido)acetate;
3-(4-(((4-cyanobenzyl)(2-methyl-3-(methylsulfonamido)phenyl)amino)methyl)phenoxy)-N-(pyridin-2-ylmethyl)-5-((tetrahydrofuran-3-yl)methoxy)benzamide;
N-(3-((3-fluorobenzyl)(4-(3-((tetrahydrofuran-3-yl)methoxy)phenoxy)benzyl)amino)-2-methylphenyl)methanesulfonamide;
N-(3-((3-chlorobenzyl)(4-(3-((tetrahydrofuran-3-yl)methoxy)phenoxy)benzyl)amino)-2-methylphenyl)methanesulfonamide;
N-(3-((3-cyanobenzyl)(4-(3-(pyridin-3-ylmethoxy)phenoxy)benzyl)amino)-2-methylphenyl)ethanesulfonamide;
N-(3-((4-chlorobenzyl)(4-(3-((tetrahydrofuran-3-yl)methoxy)phenoxy)benzyl)amino)-2-methylphenyl)methanesulfonamide;
N-(3-((3-fluorobenzyl)(4-(3-(pyridin-3-ylmethoxy)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-N,N-dimethyl-5-(2-(pyridin-3-yl)ethoxy)benzamide;
3-(4-(((4-cyanobenzyl)(2-methyl-3-(methylsulfonamido)phenyl)amino)methyl)phenoxy)-5-(pyridin-3-ylmethoxy)benzamide;
3-(4-(((4-cyanobenzyl)(2-methyl-3-(methylsulfonamido)phenyl)amino)methyl)phenoxy)-N-methyl-5-((tetrahydrofuran-3-yl)methoxy)benzamide;
N-(2-methyl-3-((4-methylbenzyl)(4-(3-((tetrahydrofuran-3-yl)methoxy)phenoxy)benzyl)amino)phenyl)methanesulfonamide;
N-(3-((4-cyanobenzyl)(4-(3-(pyridin-3-ylmethoxy)phenoxy)benzyl)amino)-2-methylphenyl)ethanesulfonamide;
N-(3-((3-cyanobenzyl)(4-(3-(pyridin-3-ylmethoxy)phenoxy)benzyl)amino)-2-methylphenyl)methanesulfonamide;
N-(3-((3-cyanobenzyl)(4-(3-((tetrahydrofuran-3-yl)methoxy)phenoxy)benzyl)amino)-2-methylphenyl)methanesulfonamide;
N-(3-((3-fluorobenzyl)(4-(3-(2-(pyridin-3-yl)ethoxy)phenoxy)benzyl)amino)-2-methylphenyl)methanesulfonamide;
N-(3-((4-cyanobenzyl)(4-(4-fluoro-3-(2-(pyridin-3-yl)ethoxy)phenoxy)benzyl)amino)-2-methylphenyl)methanesulfonamide;
N-(3-((4-cyanobenzyl)(4-(3-((tetrahydrofuran-3-yl)methoxy)phenoxy)benzyl)amino)-2-methylphenyl)methanesulfonamide;
N-(3-((4-cyanobenzyl)(4-(3-(pyridin-3-ylmethoxy)phenoxy)benzyl)amino)-2-methylphenyl)methanesulfonamide;
N-(3-((3-cyanobenzyl)(4-(3-(2-(pyridin-3-yl)ethoxy)phenoxy)benzyl)amino)-2-methylphenyl)ethanesulfonamide;
3-(4-(((4-cyanobenzyl)(2-methyl-3-(methylsulfonamido)phenyl)amino)methyl)phenoxy)-5-((tetrahydrofuran-3-yl)methoxy)benzamide;
N-(3-((3-cyanobenzyl)(4-(3-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)-3-fluorophenoxy)-5-((tetrahydrofuran-3-yl)methoxy)benzamide;
N-(3-((4-cyanobenzyl)(4-(3-(2-(pyridin-3-yl)ethoxy)phenoxy)benzyl)amino)-2-methylphenyl)ethanesulfonamide;
3-(4-(((4-cyanobenzyl)(2-methyl-3-(methylsulfonamido)phenyl)amino)methyl)phenoxy)-N-cyclopropyl-5-(2-(pyridin-3-yl)ethoxy)benzamide;
3-(4-(((4-cyanobenzyl)(2-methyl-3-(methylsulfonamido)phenyl)amino)methyl)phenoxy)-N-(pyridin-2-ylmethyl)-5-(2-(pyridin-3-yl)ethoxy)benzamide;
3-(4-(((4-cyanobenzyl)(2-methyl-3-(methylsulfonamido)phenyl)amino)methyl)phenoxy)-5-(2-(pyridin-3-yl)ethoxy)-N-(pyridin-4-ylmethyl)benzamide;
3-(4-(((4-cyanobenzyl)(2-methyl-3-(methylsulfonamido)phenyl)amino)methyl)phenoxy)-5-(2-(pyridin-3-yl)ethoxy)-N-(pyridin-3-ylmethyl)benzamide;
N-(3-((4-cyanobenzyl)(4-(3-(2-(pyridin-3-yl)ethoxy)phenoxy)benzyl)amino)-2-methylphenyl)methanesulfonamide;
ethyl 2-(3-(4-(((4-cyanobenzyl)(2-methyl-3-(methylsulfonamido)phenyl)amino)methyl)phenoxy)-5-(2-(pyridin-3-yl)ethoxy)benzamido)acetate;
3-(4-(((4-cyanobenzyl)(2-methyl-3-(methylsulfonamido)phenyl)amino)methyl)phenoxy)-N-ethyl-5-(2-(pyridin-3-yl)ethoxy)benzamide;
N-(3-((4-cyanobenzyl)(4-(3-(hydrazinecarbonyl)-5-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-N-methoxy-5-(2-(pyridin-3-yl)ethoxy)benzamide;
3-(4-(((4-cyanobenzyl)(2-methyl-3-(methylsulfonamido)phenyl)amino)methyl)phenoxy)-N-methyl-5-(2-(pyridin-3-yl)ethoxy)benzamide;
3-(4-(((4-cyanobenzyl)(2-methyl-3-(methylsulfonamido)phenyl)amino)methyl)phenoxy)-5-(2-(pyridin-3-yl)ethoxy)benzamide;
N-(3-((4-cyanobenzyl)(4-(3-(hydroxymethyl)-5-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)-2-fluorophenoxy)-5-((tetrahydrofuran-3-yl)methoxy)benzamide;
N-(3-(benzyl(4-(3-(2-(pyridin-3-yl)ethoxy)phenoxy)benzyl)amino)-2-methylphenyl)methanesulfonamide;
N-(3-((4-cyanobenzyl)(4-(3-(hydroxymethyl)-5-(pyridin-3-ylmethoxy)phenoxy)benzyl)amino)-2-methylphenyl)methanesulfonamide;
N-(3-((3-cyanobenzyl)(4-(3-(hydroxymethyl)-5-((tetrahydrofuran-3-yl)methoxy)phenoxy)benzyl)amino)-2-methylphenyl)methanesulfonamide;
N-(3-((4-cyanobenzyl)(4-(3-(hydroxymethyl)-5-((tetrahydrofuran-3-yl)methoxy)phenoxy)benzyl)amino)-2-methylphenyl)methanesulfonamide;
N-(3-((4-(3-cyano-5-((tetrahydrofuran-3-yl)methoxy)phenoxy)benzyl)(4-cyanobenzyl)amino)-2-methylphenyl)methanesulfonamide;
3-(4-(((4-cyano-3-fluorobenzyl)(2-methyl-3-(methylsulfonamido)phenyl)amino)methyl)phenoxy)-5-(2-(pyridin-3-yl)ethoxy)benzamide;
N-(3-((4-(3-cyano-5-(2-(pyridin-3-yl)ethoxy)phenoxy)benzyl)(4-cyanobenzyl)amino)-2-methylphenyl)methanesulfonamide;
N-(3-((4-cyanobenzyl)(4-(3-(hydroxymethyl)-5-(pyridin-3-ylmethoxy)phenoxy)benzyl)amino)-2-methylphenyl)ethanesulfonamide;
N-(3-((3-cyanobenzyl)(4-(3-(hydroxymethyl)-5-((tetrahydrofuran-3-yl)methoxy)phenoxy)benzyl)amino)-2-methylphenyl)ethanesulfonamide;
N-(3-(benzyl(4-(3-((tetrahydro-2H-pyran-3-yl)methoxy)phenoxy)benzyl)amino)-2-methylphenyl)methanesulfonamide;
N-(3-(benzyl(4-(3-(3-(methylthio)propoxy)phenoxy)benzyl)amino)-2-methylphenyl)methanesulfonamide;
N-(3-(benzyl(4-(3-(pyridin-3-yloxy)phenoxy)benzyl)amino)-2-methylphenyl)methanesulfonamide;
N-(3-(benzyl(4-(3-(2-(pyridin-4-yl)ethoxy)phenoxy)benzyl)amino)-2-methylphenyl)methanesulfonamide;
N-(3-((4-(3-(2-(1H-pyrazol-1-yl)ethoxy)phenoxy)benzyl)(benzyl)amino)-2-methylphenyl)methanesulfonamide;
N-(3-(benzyl(4-(3-(2-(isoxazol-5-yl)ethoxy)phenoxy)benzyl)amino)-2-methylphenyl)methanesulfonamide;
N-(3-(benzyl(4-(3-(2-(furan-2-yl)ethoxy)phenoxy)benzyl)amino)-2-methylphenyl)methanesulfonamide;
N-(3-(benzyl(4-(3-(thiazol-5-ylmethoxy)phenoxy)benzyl)amino)-2-methylphenyl)methanesulfonamide;
N-(3-(benzyl(4-(3-(oxazol-4-ylmethoxy)phenoxy)benzyl)amino)-2-methylphenyl)methanesulfonamide;
N1-(4-(4-(((4-cyanobenzyl)(2-methyl-3-(methylsulfonamido)phenyl)amino)methyl)phenoxy)-2-(2-(pyridin-3-yl)ethoxy)phenyl)-N3-ethylmalonamide;
N1-(4-(4-(((4-cyanobenzyl)(2-methyl-3-(methylsulfonamido)phenyl)amino)methyl)phenoxy)-2-(2-(pyridin-3-yl)ethoxy)phenyl)-N3-methylmalonamide;
N-(3-((4-cyanobenzyl)(4-(4′-hydroxybiphenyl-3-yloxy)benzyl)amino)-2-methylphenyl)methanesulfonamide;
3-(3-(4-(((4-cyanobenzyl)(2-methyl-3-(methylsulfonamido)phenyl)amino)methyl)phenoxy)-5-(2-(pyridin-3-yl)ethoxy)phenylamino)-3-oxopropanoic acid;
N-(3-((4-cyanobenzyl)(4-(3-(2-(furan-2-yl)ethoxy)phenoxy)benzyl)amino)-2-methylphenyl)methanesulfonamide;
N-(3-((4-cyanobenzyl)(4-(2-(2-(pyridin-3-yl)ethoxy)pyridin-4-yloxy)benzyl)amino)-2-methylphenyl)methanesulfonamide;
N-(3-((3-cyanobenzyl)(4-(3-(2-(furan-2-yl)ethoxy)phenoxy)benzyl)amino)-2-methylphenyl)methanesulfonamide;
N-(3-((4-cyanobenzyl)(4-(5-(2-(pyridin-3-yl)ethoxy)pyridin-3-yloxy)benzyl)amino)-2-methylphenyl)methanesulfonamide;
(E)-N-(3-((4-cyanobenzyl)(4-(3-(2-(pyridin-3-yl)vinyl)phenoxy)benzyl)amino)-2-methylphenyl)methanesulfonamide;
N-(3-((4-cyanobenzyl)(4-(3-(2-(pyridin-3-yl)ethyl)phenoxy)benzyl)amino)-2-methylphenyl)methanesulfonamide;
N-(3-((4-cyanobenzyl)(4-(3-((pyridin-3-ylmethoxy)methyl)phenoxy)benzyl)amino)-2-methylphenyl)methanesulfonamide;
N-(3-((4-cyanobenzyl)(4-(3-((thiazol-5-ylmethoxy)methyl)phenoxy)benzyl)amino)-2-methylphenyl)methanesulfonamide;
N-(3-((4-cyanobenzyl)(4-(2-fluoro-6-(2-(pyridin-3-yl)ethoxy)pyridin-4-yloxy)benzyl)amino)-2-methylphenyl)methanesulfonamide;
N-(3-((4-cyanobenzyl)(4-(2-(propylthio)-6-(2-(pyridin-3-yl)ethoxy)pyridin-4-yloxy)benzyl)amino)-2-methylphenyl)methanesulfonamide;
N-(3-((4-(3-cyano-5-((tetrahydrofuran-3-yl)methoxy)phenoxy)-3-fluorobenzyl)(4-cyanobenzyl)amino)-2-methylphenyl)methanesulfonamide;
N-(3-((4-(3-cyano-5-(pyridin-3-ylmethoxy)phenoxy)benzyl)(4-cyanobenzyl)amino)-2-methylphenyl)methanesulfonamide;
   or a pharmaceutically acceptable salt thereof.

Further compounds of formula (I) include, but are not limited to, the following compounds:
N-(3-((4-cyanobenzyl)(4-(3-(hydroxymethyl)-5-((tetrahydrofuran-3-yl)methoxy)phenoxy)benzyl)amino)-2-methylphenyl)ethanesulfonamide;
N-(3-((3-cyanobenzyl)(4-(3-(hydroxymethyl)-5-(pyridin-3-ylmethoxy)phenoxy)benzyl)amino)-2-methylphenyl)methanesulfonamide.

In the compounds listed above and in the Examples, the compound names were generated in accordance with IUPAC by the ACD Labs 8.0/name program, version 8.05 and/or with ISIS DRAW Autonom 2000 and/or ChemBioDraw Ultra version 11.0.

The compounds of the invention have been found to be ligands of the glucocorticoid receptor. Preferred compounds of the invention are those which display significant IL-6 activity, particularly compounds which display IL-6 absolute potency of 5 μM or less. Such compounds are considered to have potential as anti-inflammatory compounds. Further preferred compounds of the invention are those which display IL-6 absolute potency of 70 nM or less.

The compounds of the invention also display dissociation between anti-inflammatory and metabolic actions and thereby are expected to show the desired beneficial profile of dissociated glucocorticoid ligands. Dissociation is defined as the ratio between the absolute EC50 (IC50) for the side effect and the absolute EC50 (IC50) for the anti-inflammatory effect. Here, absolute EC50 (or IC50) is defined as the concentration of test compound at which it exerts an effect equal to 50% of the saturation effect achieved with dexamethasone in the same test occasion. Preferred compounds of the invention are those which display dissociation against tyrosine aminotransferase (TAT) of 5 times or more, which is better than the most TAT dissociated compound we are aware of in WO 2002/064550. Alternative preferred compounds of the invention are those which display an improved osteocalcin (OC) versus anti-inflammatory profile which is better than the best compound we are aware of in WO 2002/064550.

The compounds of the invention may display either favourable IL-6 potency or a favourable dissociation profile, or they may display both favourable potency and dissociation properties. For example, preferred compounds of the invention may display dissociation against tyrosine aminotransferase (TAT) of 5 times or more, or they may display IL-6 absolute potency of 70 nM or less. Further preferred compounds may display both dissociation against tyrosine aminotransferase (TAT) of 5 times or more and IL-6 absolute potency of 70 nM or less. Alternative preferred compounds of the invention are those which either display IL-6 absolute potency of 70 nM or less, or display TAT dissociation of 5 times or more, or display an improved osteocalcin (OC) versus anti-inflammatory profile which is better than the best compound we are aware of in WO 2002/064550.

The compounds of the invention may contain chiral (asymmetric) centers or the molecule as a whole may be chiral. The individual stereoisomers (enantiomers and diastereoisomers) and mixtures of these are within the scope of the present invention.

The novel compounds of the present invention can be formulated into suitable pharmaceutically acceptable compositions by combining with suitable excipients by techniques and processes and concentrations as are well known. Accordingly, in another aspect of the invention, there is provided a pharmaceutical composition which comprises a compound of formula (I), together with a pharmaceutically acceptable carrier. Whilst a compound of the invention may be used as the sole active ingredient in a pharmaceutical composition or medicament, it is also possible for the compound to be used in combination with one or more further active agents. Such further active agents may be further compounds according to the invention, or they may be different therapeutic agents. Accordingly, the present invention provides a pharmaceutical composition which comprises a compound of formula (I), together with a pharmaceutically acceptable carrier, and together with one or more further therapeutic agents.

In another aspect of the invention, there is provided a compound of formula (I), for use as a medicament. In one embodiment of this aspect, said compound is for use in the treatment or prophylaxis of a condition associated with a disease or disorder for which glucocorticoid treatment is indicated.

In another aspect of the invention, there is provided use of a compound of formula (I), for the manufacture of a medicament for the treatment or prophylaxis of a condition associated with a disease or disorder for which glucocorticoid treatment is indicated.

In another aspect of the invention, there is provided a method for the treatment or prophylaxis in a mammal of a disease or disorder for which glucocorticoid treatment is indicated, which comprises administering to the mammal a therapeutically effective amount of a compound of formula (I).

The compounds of formula (I) or pharmaceutical compositions containing them are useful as ligands of the glucocorticoid receptors suitable for humans and other warm blooded animals, and may be administered either by oral, topical or parenteral administration. The compounds of the present invention have suitable anti-inflammatory properties. The compounds of the present invention have reduced side effects compared to the currently used glucocorticoids. The compounds of the present invention show dissociation in the transactivation and transrepression pathways, and therefore show a reduced side effect profile. Thus, the compounds of the present invention are suitable for the treatment and/or mitigation and control of inflammatory, immune, and allergic disorders including rheumatic diseases such as rheumatoid arthritis, juvenile arthritis, Sjögren's syndrome, and ankylosing spondylitis, dermatological diseases including psoriasis and pemphigus, allergic disorders including allergic rhinitis, atopic dermatitis, and contact dermatitis, pulmonary conditions including asthma and chronic obstructive pulmonary disease (COPD), and other immune and inflammatory diseases including Crohn disease, ulcerative colitis, systemic lupus erythematosus, autoimmune chronic active hepatitis, osteoarthritis, tendonitis, and bursitis.

The compounds of the present invention are also suitable for the treatment cerebral and intracranial oedema, malignancies such as lymphomas and leukaemia, autoimmune disorders such as multiple sclerosis and amyotrophic lateral sclerosis, pain and in transplant rejection suppression.

The compounds of the present invention are suitable for treating diseases characterized by inflammatory, allergic, or proliferative processes, in a patient in need of such treatment. Accordingly, in another aspect of the invention, there is provided a method for the treatment or prophylaxis of a disease or disorder associated with glucocorticoid receptor activity in a mammal, which comprises administering to the mammal a therapeutically effective amount of a compound of formula (I), or use of a compound of formula (I), for the manufacture of a medicament for the treatment or prophylaxis of a condition associated with a disease or disorder associated with glucocorticoid receptor activity, wherein said disease or disorder is selected from inflammation and arthritis.

The pharmaceutical composition is provided by employing conventional techniques. Preferably the composition is in unit, dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration.

The following definitions apply to the terms as used throughout this specification, unless otherwise limited in specific instances.

As used herein, the term “substituted”, particularly when used in the phrase “optionally substituted”, when used in combination with other radicals, denotes suitable substituents on that radical such as substituted alkyl, substituted alkenyl, substituted alkynyl, substituted cycloalkyl, substituted aryl, etc, mentioned anywhere in the specification. The suitable substituent include, but are not limited to the following radicals, alone or in combination with other radicals, such as, hydroxyl, oxo, halo, thio, nitro, amino, cyano, formyl, hydrazino, alkyl, haloalkyl, alkoxy, haloalkoxy, alkenyl, alkynyl, cycloalkyl, cycloalkenyl, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkoxycarbonylamino, aryloxycarbonylamino, aralkyloxycarbonylamino, aminocarbonylamino, alkylaminocarbonylamino, alkoxyamino, hydroxylamino, sulfonyl derivatives, sulfonic acid and its derivatives, phosphonic acid and its derivatives.

The term “alkyl” used herein, either alone or in combination with other radicals, denotes a linear or branched radical containing one to twelve carbons, such as methyl, ethyl, n-propyl, iso-propyl, n-butyl, sec-butyl, t-butyl, amyl, t-amyl, n-pentyl, n-hexyl, iso-hexyl, heptyl, octyl and the like.

The term “alkenyl” used herein, either alone or in combination with other radicals, denotes a linear or branched radical containing two to twelve carbons; such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

The term “cycloalkenyl” used herein, either alone or in combination with other radicals, denotes a radical containing three to seven carbons, such as cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like.

The term “alkoxy” used herein, either alone or in combination with other radicals, denotes a radical alkyl, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes, an alkenyl radical, as defined above, attached to an oxygen atom, such as vinyloxy, allyloxy, butenoxy, pentenoxy, hexenoxy, and the like.

The term “cycloalkoxy” used herein, either alone or in, combination with other radicals, denotes a radical containing three to seven carbon atoms, as defined above, attached directly to an oxygen atom, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl” etc refers to a fluoro, chloro, bromo or iodo group. The term “haloalkyl” denotes an alkyl radical, as defined above, substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like.

The term “aryl” or “aromatic” used herein, either alone or in combination with other radicals, denotes an aromatic system containing one, two or three rings wherein such rings may be attached together in a pendant manner or may be fused, such as phenyl, naphthyl, tetrahydronaphthyl, indane, biphenyl, and the like. The term ‘aralkyl” denotes an alkyl group, as defined above, attached to an aryl, such as benzyl, phenethyl, naphthylmethyl, and the like.

The term “aryloxy” denotes an aryl radical, as defined above, attached to an alkoxy group, as defined above, such as phenoxy, naphthyloxy and the like, which may be substituted. The term “aralkoxy” denotes an arylalkyl moiety, as defined above, attached directly to an oxygen atom, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, or partially saturated ring-shaped radicals, the heteroatoms selected from nitrogen, sulfur and oxygen. Examples of saturated heterocyclic radicals include but not limited to aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, and the like.

The term “heteroaryl” or “heteroaromatic” used herein, either alone or in combination with other radicals, denotes unsaturated, aromatic 5- to 6-membered heterocyclic radicals containing one or more hetero atoms selected from O, N or S, such as pyridyl, thienyl, furyl, pyrrolyl, oxazolyl, thiazolyl, imidazolyl, oxadiazolyl, tetrazolyl, benzopyranyl, benzopyranyl, benzothienyl, indolinyl, indolyl, quinolinyl, pyrimidinyl, pyrazolyl, quinazolinyl, pyrimidonyl, benzoxazinyl, benzoxazinonyl, benzothiazinyl, benzothiazinonyl, benzoxazolyl, benzothizaolyl, benzimidazolyl, and the like.

The term “heterocyclylalkyl” used herein, either alone or in combination with other radicals, represents a heterocyclyl group, as defined above, substituted with an alkyl group of one to twelve carbons, such as pyrrolidinealkyl, piperidinealkyl, morpholinealkyl, thiomorpholinealkyl, oxazolinealkyl, and the like, which may be substituted. The term “heteroaralkyl” used herein, either alone or in combination with other radicals, denotes a heteroaryl group, as defined above, attached to a straight or branched saturated carbon chain containing 1 to 6 carbons, such as (2-furyl)methyl, (3-furyl)methyl, (2-thienyl)methyl, (3-thienyl)methyl, (2-pyridyl)methyl, 1-methyl-1-(2-s pyrimidyl)ethyl and the like. The terms “heteroaryloxy”, “heteroaralkoxy”, “heterocycloxy”, “heterocylylalkoxy” denotes heteroaryl, heteroarylalkyl, heterocyclyl, heterocyclylalkyl groups respectively, as defined above, attached to an oxygen atom.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which may be substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, attached to amino group which may be substituted, such as CH₃CONH, C₂H₅CONH, C₃H₇CONH, C₄H₉CONH, C₆H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arylalkyl groups. Examples of monoalkylamino group include methylamine, ethylamine, n-propylamine, n-butylamine, n-pentylamine and the like.

The term ‘disubstituted amino” used herein, either alone or in combination with other radicals, denotes an amino group, substituted with two radicals that may be same or different selected from (C₁-C₆)alkyl, substituted alkyl, aryl, substituted aryl, or arylalkyl groups, such as dimethylamino, methylethylamino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 2-(1-napthyl)ethylamino and the like.

The term “oxo” or “carbonyl” used herein, either alone (—C═O—) or in combination with other radicals, such as “alkylcarbonyl”, denotes a carbonyl radical (—C═O—) substituted with an alkyl radical such as acyl or alkanoyl, as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted; aralkoxycarbonyl group such as benzyloxycarbonyl, phenethyloxycarbonyl, napthylmethoxycarbonyl, and the like, which may be substituted; heteroaryloxycarbonyl, heteroaralkoxycarbonyl, wherein the heteroaryl group, is as defined above, which may be substituted; heterocyclyloxycarbonyl, where the heterocyclic group, as defined earlier, which may be substituted.

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as ‘aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “alkylthio” used herein, either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thioalkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio’ used herein, either alone or in combination with other radicals, denotes an aryl group, as defined above, linked through a divalent sulfur atom, having a free valence bond from the sulfur atom such as phenylthio, napthylthio and the like.

The term “alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O)CONH, C₆H₄(OCH₃)OCONH, and the like. The term “aralkoxycarbonylamino” used herein, alone or in combination with other radicals, denotes an aralkoxycarbonyl group, as defined above, attached to an amino group C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like.

The term “aminocarbonylamino”, “alkylaminocarbonylamino”, “dialkylaminocarbonylamino” used herein, alone or in combination with other radicals, denotes a carbonylamino (—CONH₂) group, attached to amino(NH₂), alkylamino group or dialkylamino group respectively, where alkyl group is as defined above.

The term “amidino” used herein, either alone or in combination with other radicals, denotes a —C(═NH)—NH₂ radical. The term “alkylamidino” denotes an alkyl radical, as discussed above, attached to an amidino group.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfonyl” or “sulfones and its derivatives” used herein, either alone or in combination with other radicals, with other terms such as alkylsulfonyl, denotes divalent radical —SO₂—, or RSO₂—, where R is substituted or unsubstituted groups selected from alkyl, aryl, heteroaryl, heterocyclyl, and the like. “Alkylsulfonyl” denotes alkyl radicals, as defined above, attached to a sulfonyl radical, such as methylsulfonyl, ethylsulfonyl, propylsulfonyl and the like. The term “arylsulfonyl” used herein, either alone or in combination with other radicals, denotes aryl radicals, as defined above, attached to a sulfonyl radical, such as phenylsulfonyl and the like.

The term “sulfonic acid derivatives”, used herein, either alone or in combination with other radicals, denotes —SO₃H group and its derivatives such as sulfonylamino(SO₂NH₂); N-alkylaminosulfonyl and N,N-dialkylaminosulfonyl radicals where the sulfonylamino group is substituted with one and two alkyl groups respectively, such as N-methylaminosulfonyl, N-ethylaminosulfonyl, N,N-dimethylaminosulfonyl, N-methyl-N-ethylaminosulfonyl and the like; N-arylaminosulfonyl and N-alkyl-N-arylaminosulfonyl groups where the sulfonylamino group is substituted with one aryl radical, or one alkyl and one aryl radical; —SO₃R, wherein ‘R’ represents alkyl, aryl, aralkyl groups, as defined above, which may be substituted.

Suitable groups and substituents on the groups may be selected from those described anywhere in the specification.

The compounds of present invention may be prepared according to the synthetic schemes provided below. However, alternative synthetic routes to the compounds of the present invention can be devised by any person skilled in the art and the possible synthetic routes described below do not limit the invention. Where appropriate, any initially produced compound according to the invention can be converted into another compound according to the invention by known methods.

### Scheme 1:

A compound of formula (2), or salt thereof, may be prepared as described in or in an analogous manner to the methods described in J. Med. Chem., 48(16), 5295-5304 (2005).

Benzyl compounds of general formula (2) wherein all the symbols are as described earlier may be treated with suitable alkylating agents of formula (3) wherein all the symbols are as described earlier and Y is a suitable leaving group such as halides, mesylates, tosylates or triflates, and a base such as diisopropylethylamine to provide dibenzyl compounds of general formula (4) wherein all the symbols are as described earlier. Deprotection of allyl group of (4) is carried out with suitable deprotecting agents such as tetrakis (triphenylphosphine) palladium (0) and potassium carbonate in alcoholic solvents like MeOH or EtOH to get the phenol compound of general formula (5) wherein all the symbols are as described earlier. Alkylation of phenol derivative (5) is carried out with LaY wherein La and ‘Y’ are as defined earlier, in presence of a suitable alkali base such as potassium carbonate or cesium carbonate in suitable aprotic solvents such as DMF, toluene and xylene to get the compound of general formula (6) wherein all the symbols are as described earlier. Reduction of nitro group of formula (6) may be carried out either using a metal like iron or zinc in the presence of ammonium chloride or by hydrogenation using a catalyst containing Pd, Rh or Pt to provide diamino compound of general formula (7) wherein all the symbols are as described earlier. Compound (7) may be sulfonylated with suitable sulfonyl chloride derivative of general formula (8) wherein all the symbols are as described earlier in pyridine to provide the title compound of general formula (I), wherein all the symbols are as described earlier.

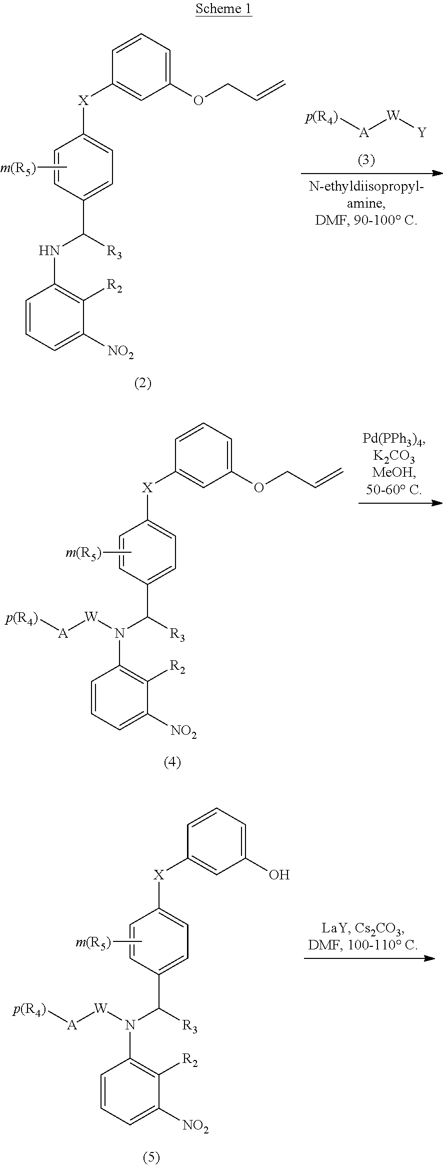

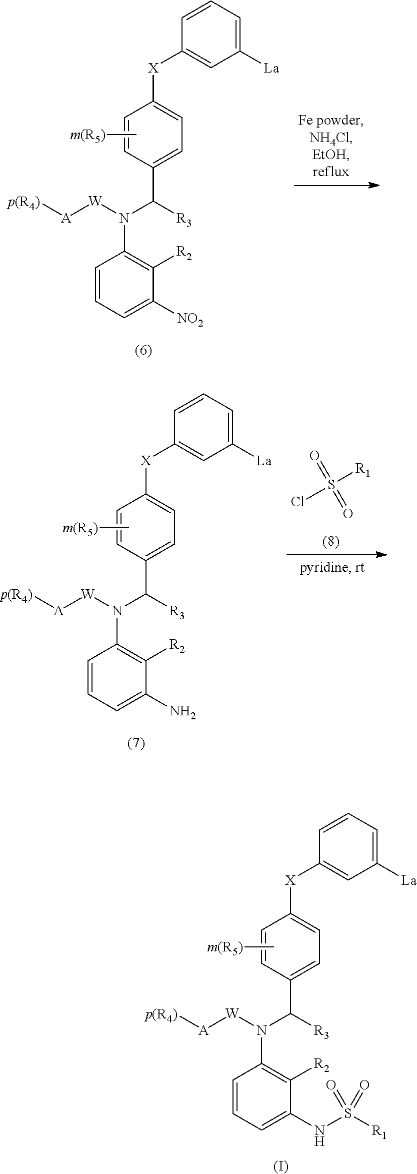

Alternatively, some of the compounds of formula (I) may be prepared by the process described by Scheme 2 (such as Examples 78, 87, 88, 90-95, 100 and 102).

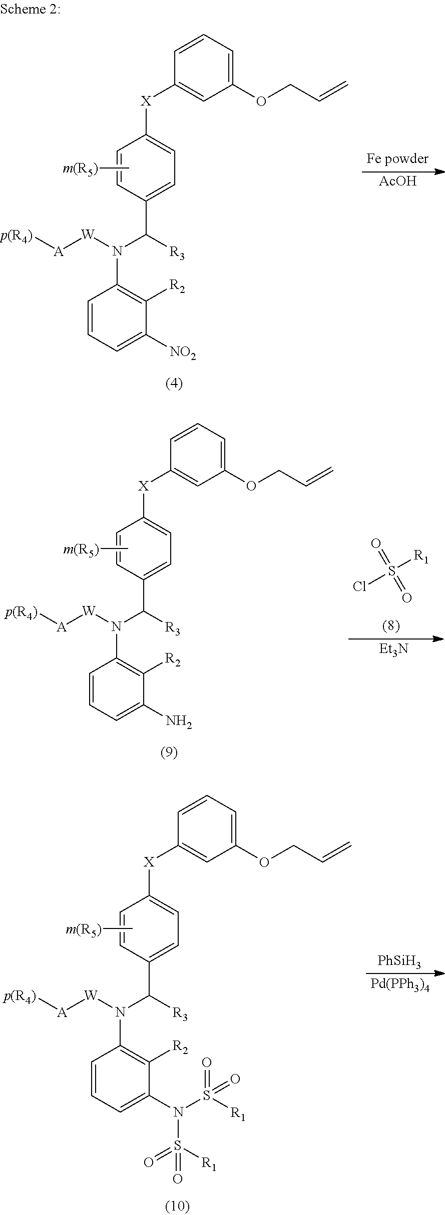

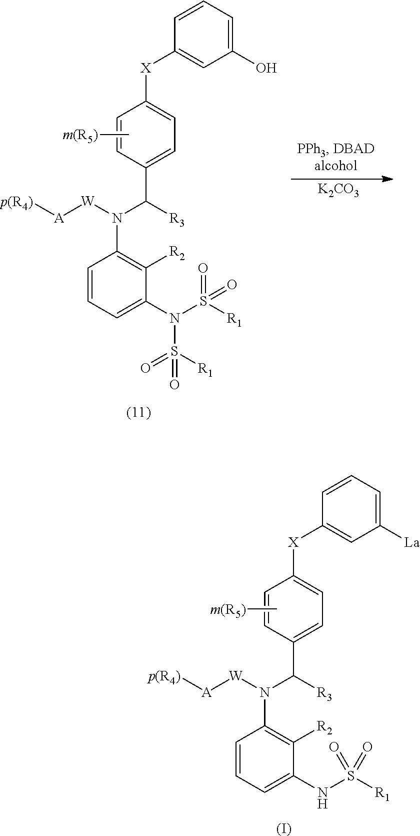

Compound (4) prepared as described in Scheme 1 may be reduced to diamino compound of general formula (9), wherein all the symbols are as described earlier, using powdered metals like iron or zinc in acidic media such as ammonium chloride solution or acetic acid. Sulfonylation of compound (9) may be performed with suitable sulfonyl chloride derivative of general formula (8), wherein all the symbols are as described earlier, in aprotic solvent like dichloromethane or THF using tertiary amines like Hünig's base or triethylamine as base providing N,N-disulfonylarylamine of general formula (10). Removal of allyl group of (10) may be carried out using tetrakis (triphenylphosphine) palladium (0) and phenylsilane providing phenol compound of general formula (11) wherein all the symbols are as described earlier. Certain compounds of formula (I) may be generated from (11) and corresponding alcohols under Mitsunobu conditions using free or resin-bound triphenyl phosphine and azodicarboxylate like DEAD or di-t-butylazodicarboxylate in suitable solvent like dichloromethane or THF.

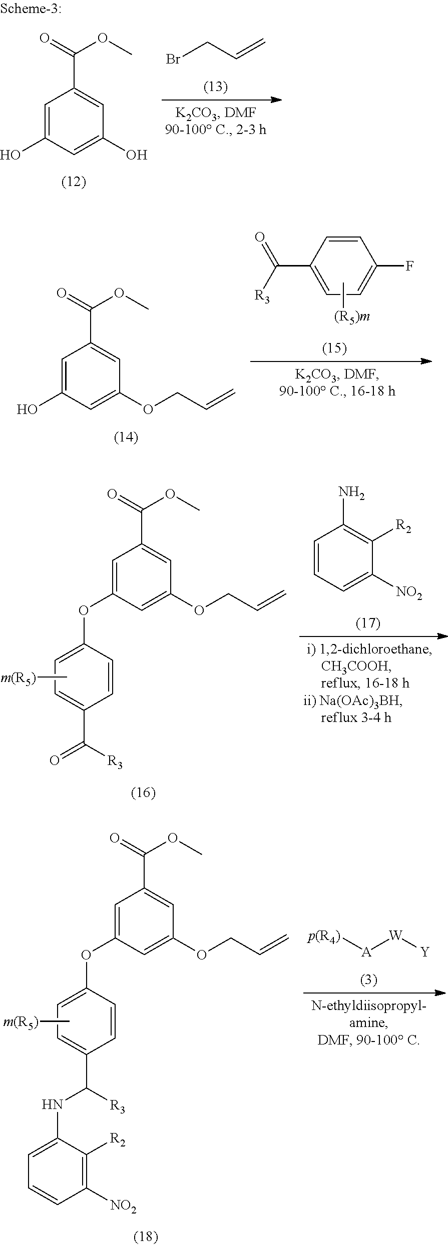

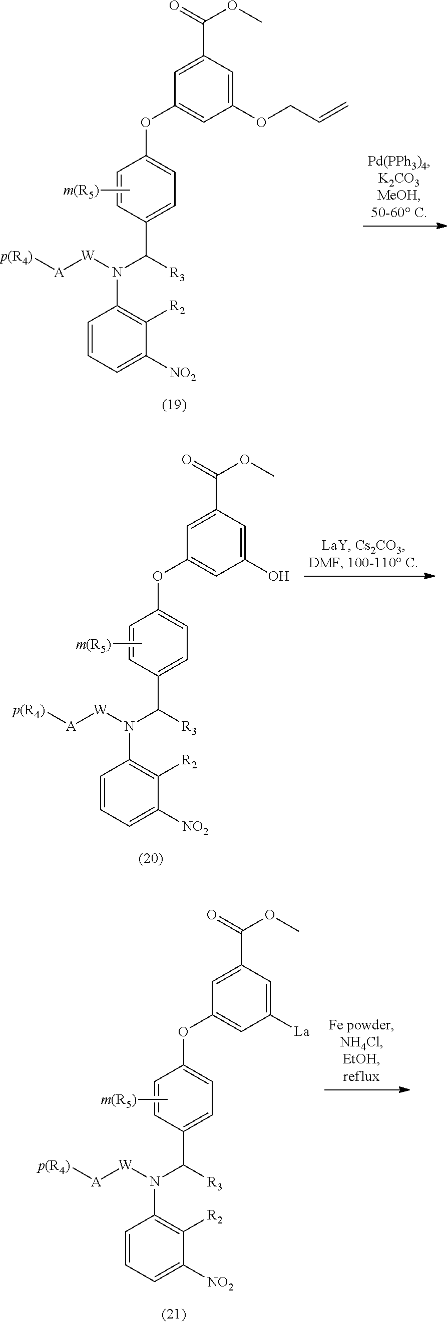

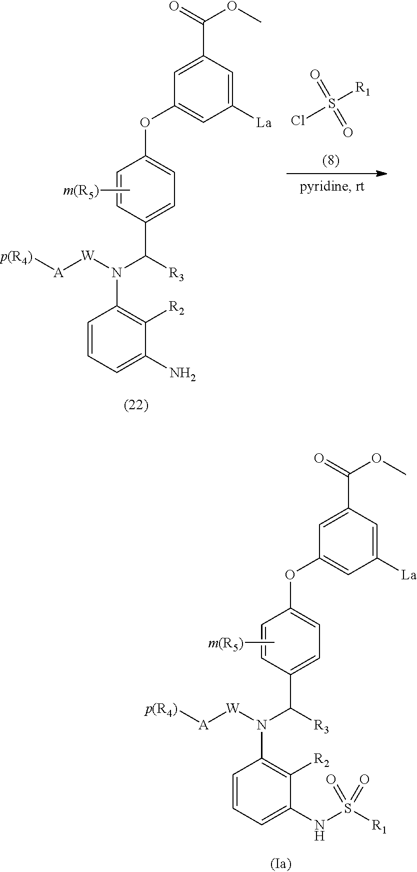

Few of the polar analogues of compound (I) may be prepared by scheme 3 and 4. Methyl 3,5-dihydroxybenzoate (12) is treated with alkylating agents such as allyl halides (13) and a base such as potassium carbonate in a polar aprotic solvent like DMF, DMSO or mixture thereof to provide monoprotected phenols of general formula (14) wherein all the symbols are as described earlier. Compound (14) is reacted with carbonyl compound (15) wherein all the symbols are as described earlier in the presence of base such as potassium carbonate in a polar aprotic solvent like DMF, DMSO or mixture thereof to provide biphenyl ether derivatives of general formula (16), wherein all the symbols are as described earlier. Compounds of general formula (16) is then treated with aniline compounds of formula (17), wherein all the symbols are as described earlier under reductive amination conditions to get the secondary amine compounds of formula (18) wherein all the symbols are as described earlier. Reductive amination is carried out in the presence of a reducing agent like lithium aluminium hydride, sodium borohydride, sodium cyanoborohydride or sodiumtriacetoxy borohydride. Secondary amines of general formula (18) may be alkylated with suitable alkylating agents of formula (3) as described in scheme 1 to get the compounds of general formula (19) wherein all the symbols are as described earlier. The compound (19) may be processed as described in scheme 1 to get its phenol compound of general formula (20) wherein all the symbols are as described earlier. Alkylation of phenol compound (20) is carried out with LaY as mentioned in the scheme 1 to get nitro compound of formula (21) wherein all the symbols are as described earlier. The nitro compound (21) is then reduced to its aniline derivative of formula (22) wherein all the symbols are as described earlier and the aniline derivative (22) is sulfonylated by the process mentioned in the scheme 1 to get the desired compound of formula (Ia) wherein all the symbols are as described earlier.

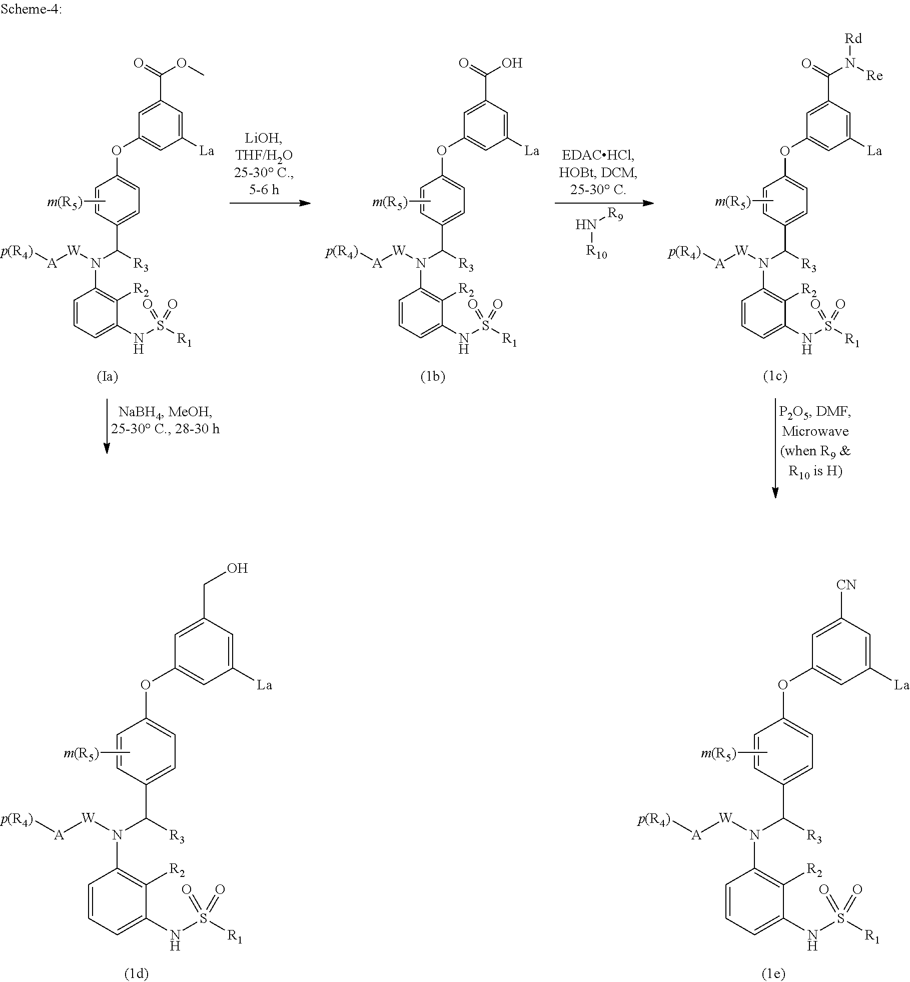

Carboxylic acid of general formula (Ib) wherein all the symbols are described as earlier is prepared by hydrolysis of compound of general formula (Ia). Hydrolysis is carried out under basic condition using base like lithium hydroxide, sodium hydroxide or potassium hydroxide in the aqueous medium. Carboxylic acid of general formula (Ib) is then reacted with suitable amine derivatives of general formula R_{d}—NH—Rₑ wherein all the symbols are described as earlier using suitable carboxyl group activating agents such as N-(3-dimethyl aminopropyl)-N′-ethyl carbodiimide hydrochloride (EDAC.HCl), dicyclohexyl carbodiimide and the like in the presence of an additive such as 1-hydroxy benzotriazole (HOBT) and a suitable base(s) like triethyl amine or diisopropylethyl amine (DIEA) in solvent(s) like DMF or DCM at temperature 0-25° C. to get the compounds of general formula (Ic) wherein all the symbols are described as earlier. In the case of R_{d}═Rₑ═H in the compounds of general formula (Ic) then these compounds are converted to its benzonitrile derivatives of general formula (Ie) wherein all the symbols are described as earlier in the presence of dehydrating agents such as P₂O₅, PCl₅, POCl₃ or like under refluxing condition. Same reaction may be carried out under microwave to get the desired benzonitrile derivatives (Ie). Compounds of general formula (Ia) may be reduced to its benzylic alcohol derivatives of general formula (Id) wherein all the symbols are described as earlier in the presence of a reducing agent like lithium aluminium hydride, sodium borohydride, sodium cyanoborohydride or sodiumtriacetoxy borohydride.

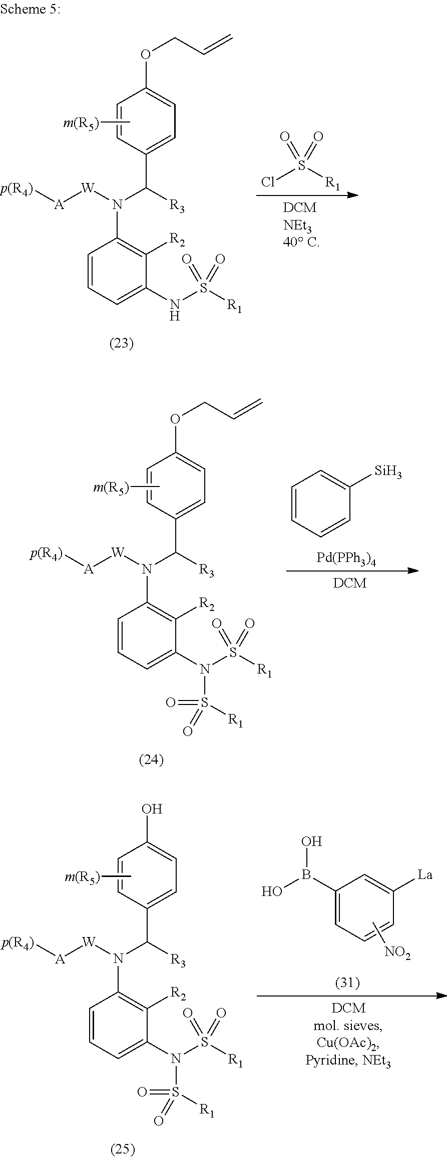

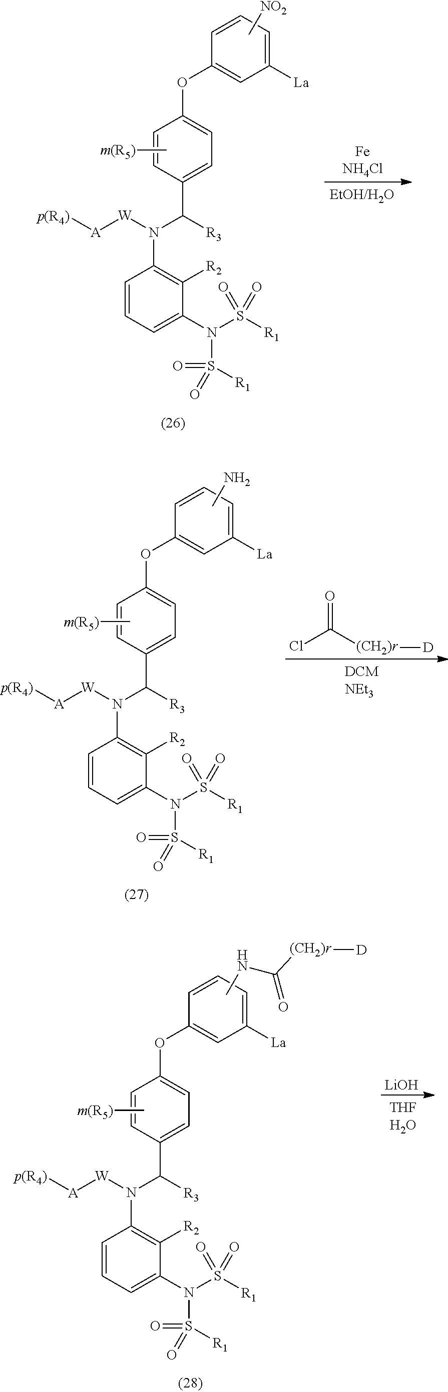

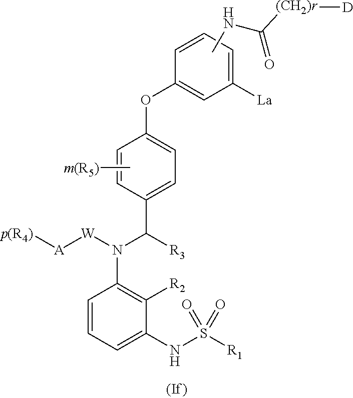

Certain compounds of formula (I) may be prepared as described in Scheme 5 (for instance, Examples 89, 96, 97, 99, 101, 103). Synthesis of compound type (23), wherein all the symbols are as described earlier, may be accomplished using methods on Scheme 1. This can be readily converted to derivatives (24) and (25) via sulfonylation and consequent removal of allyl group using tetrakis (triphenylphosphine) palladium (0) and phenylsilane as described for the compound (II) on Scheme 2. Resulting N,N-disulfonylarylamine of general formula (25) may be reacted with substituted nitrophenylboronic acids (31) to provide corresponding nitro biaryl ethers (26). This copper acetate catalysed transformation may be curried out in solvents like DCM, DMF, THF etc using suitable bases like pyridine, triethylamine or mixture of those. Molecular sieves are used to trap water and air or other oxidation media (O₂ atmosphere, aqueous H₂O₂ etc) is usually required. Compounds (26) may be reduced to amino biaryl ether compounds of general formula (27) using powdered metals like iron or zinc in acidic media as described for compound (9) on Scheme 2. Compounds of formula (27) can be acylated or sulfonylated using appropriate sulfonyl or acyl chlorides or acyl anhydrides in aprotic solvents such as DCM, THF etc and organic base like triethylamine or Hünig's base etc affording compounds of general formula (28). Mild basic hydrolysis using base like lithium hydroxide, sodium hydroxide or potassium carbonate in the aqueous THF or MeOH medium affords desired derivatives of general formula (If) bearing an Lb side-chain in which T is an —NHCO— group. Substituted nitrophenylboronic acids (31) utilized in Scheme 5 can be prepared according to Scheme 6.

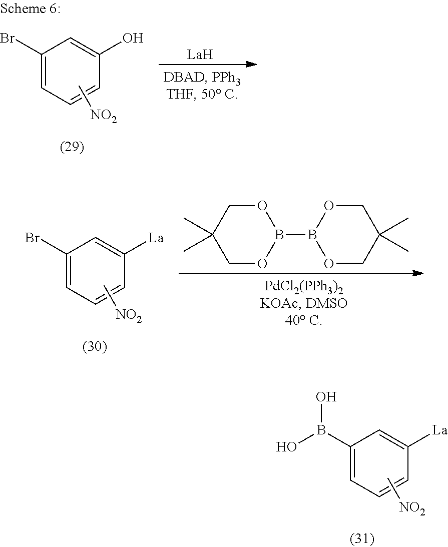

Bromo-nitrophenols (29) can be alkylated with suitable extension La using corresponding alcohols (LaH) under Mitsunobu conditions using triphenyl phosphine and azodicarboxylate like DEAD or di-t-butylazodicarboxylate in suitable solvent like dichloromethane or THF to provide compounds of general formula (30). Alternatively (29) may be treated with suitable alkylating agents of formula LaY wherein all the symbols are as described earlier and Y is a suitable leaving group such as halides, mesylates, tosylates or triflates, and a base such as diisopropylethylamine to provide compounds of general formula (30). Boronation of compounds (30) via Pd catalysed reaction with organoboronates like bis(neopentylglycolato)diboron in presence of base like potassium acetate or potassium carbonate followed by mild acidic work up like saturated aqueous NH₄Cl or 1M aqueous HCl leads to substituted nitrophenylboronic acids of general formula (31).

Compounds of type (I) bearing heterocyclic biaryl ether extensions can be made using protocol depicted in Scheme 7 (for instance, Examples 108, 109).

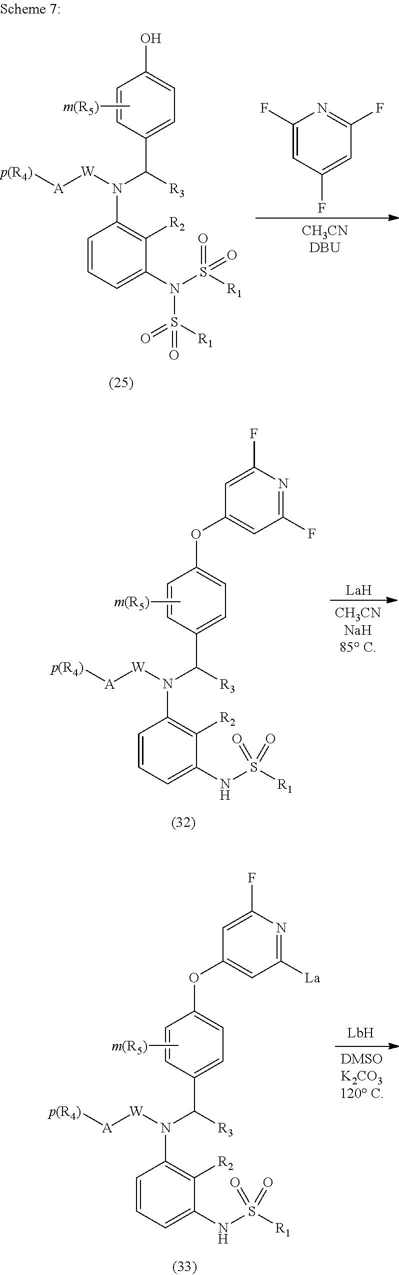

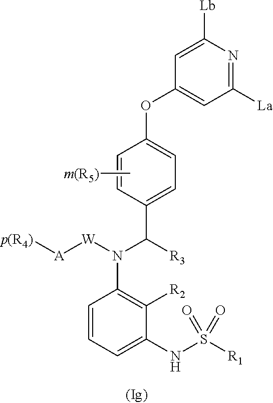

Nucleophilic aromatic substitution on appropriately substituted electron-poor heterocycles like 2,4,6-trifluoropyridine with compounds of type (25) can be achieved at mild conditions using base like DBU in aprotic solvents like DMF or acetonitrile to afford biaryl ether compounds of general formula (32). Subsequent nucleophilic aromatic substitution may be used for introduction of certain types of La extensions using corresponding alcohols (LaH) and suitable base like sodium hydride in aprotic solvent at elevated temperature to afford compounds of general formula (33). Consequent aromatic substitution with nucleophiles is possible at harsher conditions involving high heat, strong base and prolonged reaction times and leads to compounds of general formula (Ig) in which B is a pyridyl group substituted with one La side-chain and one Lb side-chain. Similar compounds of formula (I) in which B is an alternative heteroaromatic ring may be synthesised according to scheme 7 using suitable alternative electron-poor heterocycles to 2,4,6-trifluoropyridine.

Some of the compounds of general formula (I) can be made using methods presented on Scheme 8 (for instance, Examples 98, 104, 105).

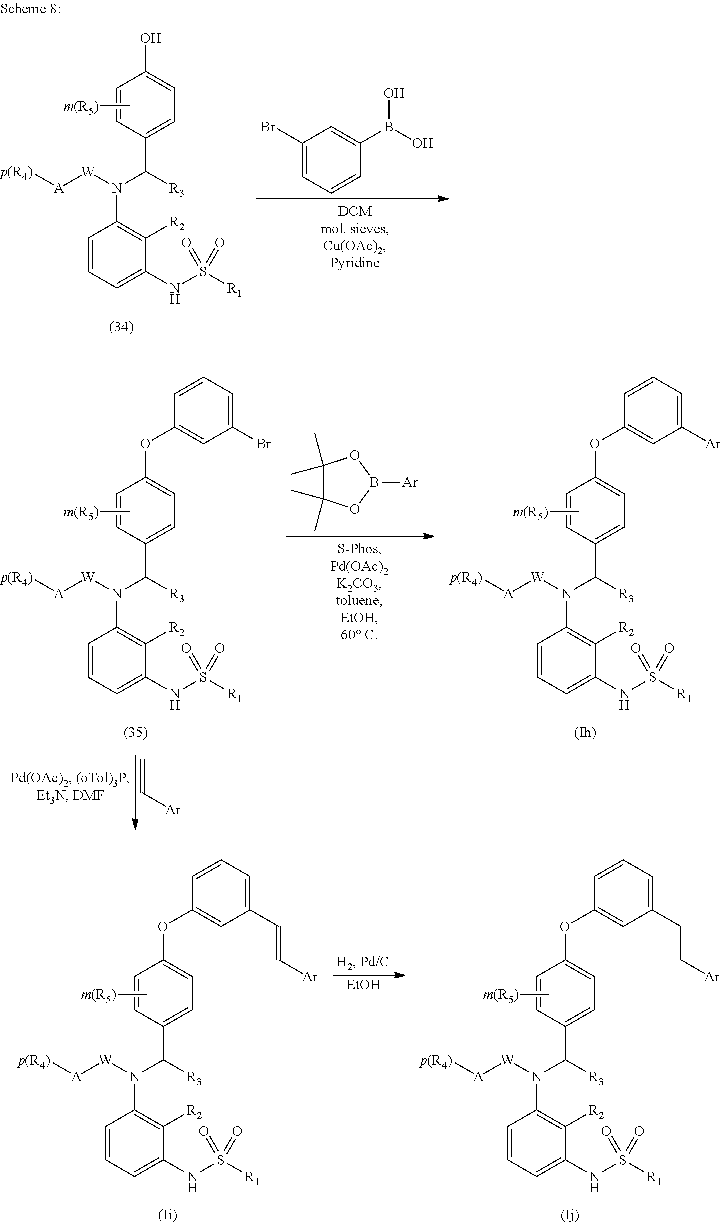

Phenol derivative of general formula (34) can be prepared as described on Scheme 1. Reactions with appropriately substituted boronic acids using same method described for compounds type (26) on Scheme 5 lead to biaryl ether derivatives of general structure (35). Subsequent cross-coupling reactions like Suzuki-Miyaura using boronic acids or esters, palladium (0) complexes with suitable ligands like triphenylphosphine or S-Phos and a base like potassium or cesium carbonate; or other alternative cross-coupling reactions such as a Stille reaction using organotin regents and suitable palladium catalyst, lead to compounds of general formula (Ih) bearing an La side-chain of the type (CH₂)q′-Z in which q′ is 0 and Z is an optionally substituted phenyl group or an optionally substituted heteroaromatic ring.

Derivatives of general structure (35) can be reacted with substituted vinylaryls under Heck coupling conditions using palladium acetate with suitable ligands like trio-tolylphosphine and base like triethylamine or Hünig's base to afford unsaturated compounds of general formula (Ii) bearing an La side-chain of the type CH═CH—Z in which Z is an optionally substituted phenyl group or an optionally substituted heteroaromatic ring. Those compounds of general formula (Ii) can be reduced using hydrogen gas and palladium on carbon as catalyst giving compounds of general formula (Ij) bearing an La side-chain of the type (CH₂)q′-Z in which q′ is 2 and Z is an optionally substituted phenyl group or an optionally substituted heteroaromatic ring.

Some of the compounds of general formula (I) can be made as depicted on Scheme 9 (such as Examples 106, 107).

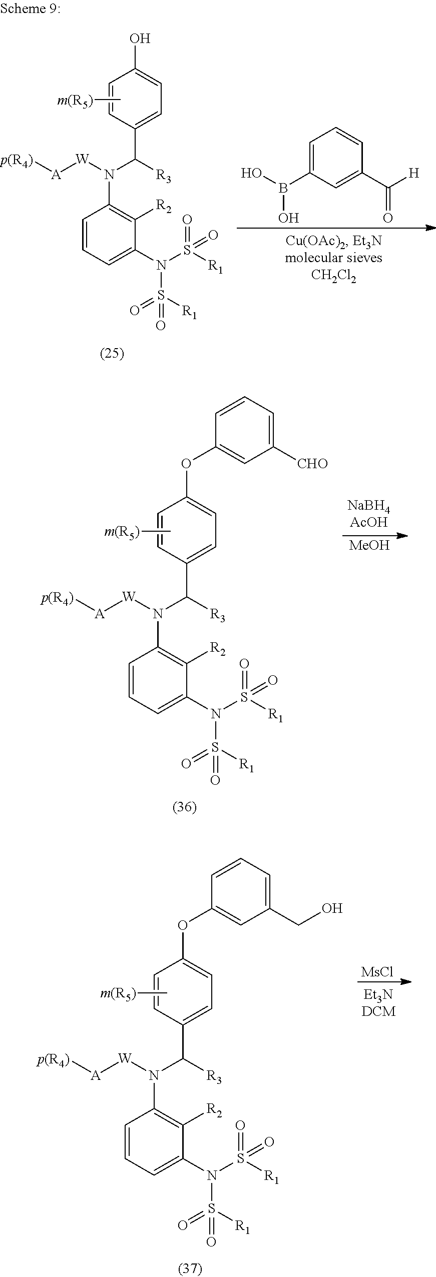

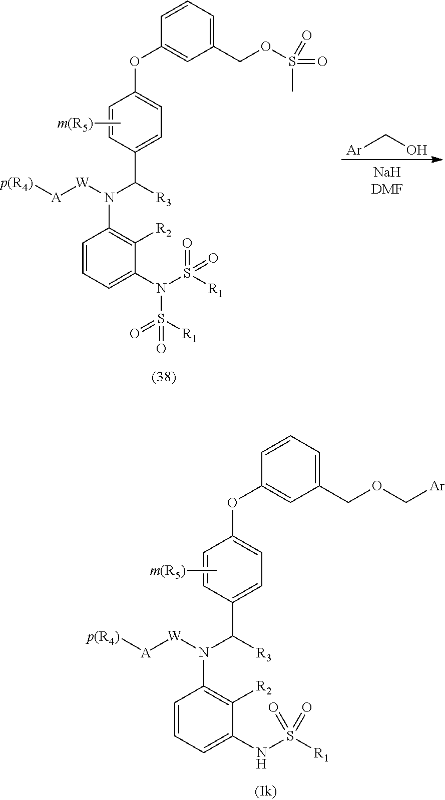

Derivatives of general formula (25) can be reacted with 3-formylphenylboronic acid under similar conditions described for preparation of compounds type (26) on Scheme 5 to provide corresponding formyl-substituted biaryl ether compounds (36). Reduction with sodium borohydride in the mixture of alcohol like methanol or ethanol and acetic acid leads to benzylic alcohol derivatives of general formula (37). Benzylic OH-group can be transformed into a suitable leaving group such as mesylate for subsequent substitution step using corresponding sulfonyl chloride or anhydride and a base such as triethylamine or diisopropylethylamine in aprotic solvent to provide intermediates of general formula (38). Mesylates of general formula (38) can be reacted with excess amounts of substituted alcohols in aprotic solvents using appropriate base like sodium hydride to afford compounds of general formula (Ik) bearing an La side-chain of the type (CH₂)r′-O—(CH₂)q′-Z in which q′ is 1, r′ is 1, and Z is an optionally substituted phenyl group or an optionally substituted heteroaromatic ring.

### EXAMPLES

The process of preparing the novel compounds of the present invention is further exemplified by the following non-limiting examples. These examples are provided for better illustration of the preferred mode of carrying out the invention and should not be construed as limiting the scope of the invention in any way. It will be appreciated that while working these examples and preparing other compounds of the present invention, the skilled person will be using the skills and diligence expected from a person skilled in the art.

### Example 15

### 3-(4-(((4-Cyanobenzyl)(2-methyl-3-(methylsulfonamido)phenyl)amino)methyl)-phenoxy)-5-(2-(pyridin-3-yl)ethoxy)benzoic acid

To the stirred solution of methyl 3-(4-(((4-cyanobenzyl)(2-methyl-3-(methylsulfonamido)phenyl]amino)methyl)phenoxy)-5-(2-(pyridin-3-yl)ethoxy)benzoate (2.0 g, 2.95 mmol) in THF (4 mL) was added solution of lithium hydroxide (0.3 g in 4 mL water) at 0-5° C. The mixture was stirred at 25-30° C. for 6 h. After completion of reaction, the reaction mixture was cooled to 0-5° C. and the pH of it was adjusted to 6.0-6.5 by adding 10% aq. HCl. Distilled out volatile solvent from it under reduced pressure. The concentrated mixture was diluted with EtOAc (100 mL) and washed it with water, brine, dried over Na₂SO₄, filtered and concentrated under reduced pressure to provide desired acid derivative which was purified by flash chromatography over silica gel (100-200 mesh) with 1.6-2.0% MeOH/CHCl₃ to provide the desired pure product (1.44 g, 74% yield) as off white solid.

¹H NMR (CDCl₃, 400 MHz) δ: 8.67 (s, 1H), 8.56 (d, J=4.0 Hz, 1H), 7.71 (d, J=7.6 Hz, 1H), 7.57 (d, J=8.4 Hz, 2H), 7.39-7.33 (m, 4H), 7.21 (d, J=8.0 Hz, 1H), 7.15 (d, J=8.0 Hz, 1H), 7.12-7.08 (m, 3H), 6.97 (d, J=8.0 Hz, 1H), 6.90-6.88 (m, 2H), 6.81 (bs, 1H), 6.72 (t, J=2.2 Hz, 1H), 4.22 (t, J=6.4 Hz, 2H), 4.14 (s, 2H), 3.99 (s, 2H), 3.12 (t, J=6.4 Hz, 2H), 2.94 (s, 3H), 2.27 (s, 3H).

### Example 35

### N-(3-((4-cyanobenzyl)(4-(3-(2-(thiophen-2-yl)ethoxy)phenoxy)benzyl)amino)-2-methylphenyl)methanesulfonamide

### Step-1: 4-(((2-methyl-3-nitrophenyl)(4-(3-(2-(thiophen-2-yl)ethoxy)phenoxy)benzyl)-amino)methyl)benzonitrile

A solution of 4-(((4-(3-hydroxyphenoxy)benzyl)(2-methyl-3-nitrophenyl)amino)methyl)benzonitrile (1.0 g, 2.15 mmol) was treated as in Example 1, step 3, with 2-(thiophen-2-yl)ethyl methanesulfonate (0.53 g, 2.58 mmol) and cesium carbonate (2.1 g, 6.45 mmol) to provide the desired pure product (0.99 g, 80% yield) as yellow oil.

¹H NMR (CDCl₃, 400 MHz) δ: 7.57 (d, J=8 Hz, 2H), 7.55-7.52 (m, 1H), 7.31 (d, J=8 Hz, 2H), 7.24-7.15 (m, 3H), 7.11-7.07 (m, 3H), 6.95-6.89 (m, 4H), 6.69-6.66 (m, 1H), 6.58-6.56 (m, 2H), 4.16-4.09 (m, 4H), 4.05 (s, 2H), 3.29 (t, J=6.4 Hz, 2H), 2.59 (s, 3H);

MS (ESI+) m/z 598.12 (M+Na)⁺.

### Step-2: 4-(((3-amino-2-methylphenyl)(4-(3-(2-(thiophen-2-yl)ethoxy)phenoxy)benzyl)amino)methyl)benzonitrile

The product of step 1 above (0.96 g, 1.67 mmol) was processed as in example 1 step 4 to provide the desired pure product (0.83 g, 92% yield) as yellow oil.

¹H NMR (CDCl₃, 400 MHz) δ: 7.53 (d, J=8.4 Hz, 2H), 7.31 (d, J=8.0 Hz, 2H), 7.23-7.14 (m, 4H), 6.95-6.87 (m, 5H), 6.67-6.64 (m, 1H), 6.58-6.56 (m, 2H), 6.46-6.41 (m, 2H), 4.15 (t, J=6.8 Hz, 2H), 4.08 (s, 2H), 3.97 (s, 2H), 3.61 (s, 2H), 3.28 (t, J=6.8 Hz, 2H), 2.42 (s, 3H);

MS (ESI+) m/z 568.1 (M+Na)⁺.

### Step-3: N-(3-((4-cyanobenzyl)(4-(3-(2-(thiophen-2-yl)ethoxy)phenoxy)benzyl)amino)-2-methylphenyl)methanesulfonamide

The product of step 2 above (0.15 g, 0.27 mmol) was processed as in example 1, step 5 to provide the desired pure product (0.12 g, 78% yield) as colorless oil.

¹H NMR (CDCl₃, 400 MHz) δ: 7.55 (d, J=8 Hz, 2H), 7.31 (d, J=8 Hz, 2H), 7.23-7.19 (m, 2H), 7.16-7.09 (m, 4H), 6.95-6.89 (m, 4H), 6.85 (d, J=8 Hz, 1H), 6.68-6.65 (m, 1H), 6.57-6.55 (m, 2H), 6.17 (s, 1H), 4.17-4.13 (m, 2H), 4.11 (s, 2H), 3.99 (s, 2H), 3.29 (t, J=6.4 Hz, 2H), 2.99 (s, 3H), 2.37 (s, 3H); MS (ESI+) m/z 646.0 (M+Na).

### Example 39

### Methyl 3-(4-(((4-cyanobenzyl)(2-methyl-3-(methylsulfonamido)phenyl)amino)methyl)phenoxy)-5-(2-(pyridin-3-yl)ethoxy)benzoate

### Step-1) Methyl 3-(allyloxy)-5-hydroxybenzoate

The mixture of 3,5-dihydroxybenzoate (25 g, 148.7 mmol), allybromide (19.8 g, 163.5 mmol) and potassium carbonate (41.0 g, 297.3 mmol) in DMF (110 mL) was heated at 80-90° C. for 1-2 h. After completion of reaction the mixture was diluted with water (300 mL) and the compound was extracted in EtOAc. The combined organic layer was washed with water, brine, dried over Na₂SO₄ filtered, and concentrated under vacuum to afford 34.1 g of crude product. Crude product was purified by flash chromatography over silica gel (100-200 mesh) with 40% EtOAc/n-hexane to provide the desired pure product (14.4 g, 47% yield) as white semisolid.

¹H NMR (CDCl₃, 400 MHz) δ: 7.199-7.19 (dd, J₁=1.6 Hz, J₂=2.4 Hz, 1H), 7.165-7.15 (dd, J₁=1.2 Hz, J₂=2.4 Hz, 1H), 6.64 (t, J=2.4 Hz, 1H), 6.08-5.98 (m, 1H), 5.76 (bs, 1H), 5.43-5.39 (dd, J₁=1.4 Hz, J₂=14.0 Hz, 1H), 5.30-5.28 (dd, J₁=1.4 Hz, J₂=9.2 Hz, 1H), 4.52 (d, J=5.2 Hz, 2H), 3.90 (s, 3H).

### Step-2) Methyl 3-(allyloxy)-5-(4-formylphenoxy)benzoate

The mixture of methyl 3-(allyloxy)-5-hydroxybenzoate (15.4 g, 74.2 mmol), 4-fluorobenzaldehyde (11.0 g, 89.0 mmol) and potassium carbonate (72.5 g, 222.5 mmol) in DMF (80 mL) was heated at 90-100° C. for 16-18 h. After completion of reaction the mixture was diluted with water (200 mL) and the compound was extracted in EtOAc (200 mL). Organic layer was washed with water, brine, dried over Na₂SO₄, filtered and concentrated under vacuum to afford 20 g of crude product. Crude product was purified by flash chromatography over silica gel (100-200 mesh) with 6% EtOAc/n-hexane to provide the desired pure product (14.8 g, 64% yield) as colourless oil.

### Step-3) methyl 3-(allyloxy)-5-(4-(((2-methyl-3-nitrophenyl)amino)methyl)phenoxy)-benzoate

The solution of methyl 3-(allyloxy)-5-(4-formylphenoxy)benzoate (14 g, 44.8 mmol), 2-methyl-3-nitroaniline (8.2 g, 53.8 mmol) and acetic acid (10.8 g, 179.5 mmol) in 1,2-dcholroethane (80 mL) was refluxed for 12-13 h under Dean-Stark apparatus to remove the water formed during the reaction. After complete formation of in-situ imine the reaction mixture was cooled to 25-30° C. and into it sodiumtriacetoxy borohydride (28.5 g, 134.6 mmol) was added portion wise within 30 minutes. The mixture was then heated under stirring at 55-60° C. for 2 h. After completion of reaction the mixture was cooled to 25-30° C. and diluted with dichloromethane (150 mL) and the organic layer was washed with water, brine, dried over Na₂SO₄, filtered and concentrated under vacuum to afford 23.2 g of crude product. Crude product was purified by flash chromatography over silica gel (100-200 mesh) with 13% EtOAc/n-hexane to provide the desired pure product (15.2 g, 75% yield) as dark yellow oil.

¹H NMR (CDCl₃, 400 MHz) δ: 7.35-7.32 (m, 3H), 7.27-7.26 (m, 1H), 7.17 (t, J=8.0 Hz, 1H), 7.11-7.09 (m, 1H), 7.02-7.00 (m, 2H), 6.78-6.76 (m, 2H), 6.06-6.00 (m, 1H), 5.43-5.40 (dd, J₁=1.2 Hz, J₂=17.2 Hz, 1H), 5.32-5.29 (dd, J=1.2 Hz, J₂=10.4 Hz, 1H), 4.55 (d, J=5.4 Hz, 2H), 4.38 (d, J=4.8 Hz, 2H), 4.18 (bt, 1H), 3.88 (s, 3H), 2.25 (s, 3H).

### Step-4) Methyl 3-(allyloxy)-5-(4-(((4-cyanobenzyl)(2-methyl-3-nitrophenyl)amino)methyl)phenoxy)benzoate

The methyl 3-(allyloxy)-5-(4-(((2-methyl-3-nitrophenyl)amino)methyl)phenoxy)-benzoate (9.5 g, 21.2 mmol) was alkylated with 4-(bromomethyl)benzonitrile (8.2 g, 63.6 mmol) by the same procedure mentioned in the example 1, step-1 to get 15.7 g of crude product. Crude product was purified by flash chromatography over silica gel (100-200 mesh) with 13% EtOAc/n-hexane to provide the desired pure product (11.6 g, 97% yield) as light yellow thick oil.

### Step-5: Methyl 3-(4-(((4-cyanobenzyl)(2-methyl-3-nitrophenyl)amino)methyl)phenoxy)-5-hydroxybenzoate

The deprotection of allyl group of methyl 3-(allyloxy)-5-(4-(((4-cyanobenzyl)(2-methyl-3-nitrophenyl)amino)methyl)phenoxy)benzoate (11.6 g, 20.0 mmol) was carried out by the same procedure mentioned in the example 1, step-2 to get the desired phenol which was purified by flash chromatography over silica gel (100-200 mesh) with 20-25% EtOAc/n-hexane to provide the desired pure product (7.98 g, 75% yield) as light yellow thick oil.

¹H NMR (CDCl₃, 400 MHz) δ: 7.59 (d, J=8.4 Hz, 2H), 7.56-7.54 (m, 1H), 7.33 (d, J=8.0 Hz, 2H), 7.28-7.27 (m, 1H), 7.23-7.18 (m, 2H), 7.12 (d, J=8.4 Hz, 3H), 6.94 (d, J=8.4 Hz, 2H), 6.68 (t, J=2.0 Hz, 1H), 5.74 (bs, 1H), 4.18 (s, 2H), 4.06 (s, 2H), 3.87 (s, 3H), 2.58 (s, 3H).

### Step-6: Methyl 3-(4-(((4-cyanobenzyl)(2-methyl-3-nitrophenyl)amino)methyl)phenoxy)-5-(2-(pyridin-3-yl)ethoxy)benzoate

The alkylation of methyl 3-(4-(((4-cyanobenzyl)(2-methyl-3-nitrophenyl)amino)methyl)phenoxy)-5-hydroxybenzoate (4.42 g, 8.45 mmol) was carried out with the same procedure as that of example 1, step-3 but using 2-(pyridin-3-yl)ethyl methanesulfonate (2.55 g, 12.68 mmol) as an alkylating agent to get the 5.52 g of crude product. Crude product was purified by flash chromatography over silica gel (100-200 mesh) with 50-55% EtOAc/n-hexane to provide the desired pure product (3.6 g, 68% yield) as light yellow oil.

¹H NMR (CDCl₃, 400 MHz) δ: 8.55 (d, J=2.0 Hz, 1H), 8.50-8.49 (dd, J₁=1.2 Hz, J₂=6.4 Hz, 1H), 7.60-7.53 (m, 3H), 7.32 (d, J=8.0 Hz, 2H), 7.30-7.29 (m, 1H), 7.25-7.22 (m, 3H), 7.19 (t, J−8.0 Hz, 1H), 7.13 (d, J=8.8 Hz, 2H), 7.08 (d, J=8.0 Hz, 1H), 6.92 (d, J=8.4 Hz, 2H), 6.72 (t, J=2.4 Hz, 1H), 4.22-4.18 (m, 4H), 4.07 (s, 2H), 3.87 (s, 3H), 3.09 (t, J=6.4 Hz, 2H), 2.60 (s, 3H).

### Step-7: Methyl 3-(4-(((3-amino-2-methylphenyl)(4-cyanobenzyl)amino)methyl)phenoxy)-5-(2-(pyridin-3-yl)ethoxy)benzoate

The reduction of nitro group of methyl 3-(4-(((4-cyanobenzyl)(2-methyl-3-nitrophenyl)amino)methyl)phenoxy)-5-(2-(pyridin-3-yl)ethoxy)benzoate (3.6 g, 5.74 mmol) was carried out with the same procedure as that of example 1, step-4 to get the desired aniline derivative (3.31 g, 96%). The same material is used for further reaction without purification.

¹H NMR (CDCl₃, 400 MHz) δ: 8.55 (d, J=1.6 Hz, 1H), 8.50-8.49 (dd, J₁=1.2 Hz, J₂=4.8 Hz, 1H), 7.62-7.60 (m, 1H), 7.53 (d, J=8.4 Hz, 2H), 7.32 (d, J=8.4 Hz, 2H), 7.28-7.22 (m, 3H), 7.19 (d, J=8.4 Hz, 2H), 6.92-6.88 (m, 3H), 6.71 (t, J=2.4 Hz, 1H), 6.47-6.42 (m, 2H), 4.20 (t, J=6.4 Hz, 2H), 4.09 (s, 2H), 3.99 (s, 2H), 3.87 (s, 3H), 3.09 (t, J=6.4 Hz, 2H), 2.23 (s, 3H).

### Step-8: methyl 3-(4-(((4-cyanobenzyl)(2-methyl-3-(methylsulfonamido)phenyl)amino)methyl)phenoxy)-5-(2-(pyridin-3-yl)ethoxy)benzoate

The methylsulfonation of methyl 3-(4-(((3-amino-2-methylphenyl)(4-cyanobenzyl)amino)methyl)phenoxy)-5-(2-(pyridin-3-yl)ethoxy)benzoate (3.31 g, 5.54 mmol) was carried out with the same procedure as that of example 1, step-5 to get the crude title compound (4.02 g). Crude product was purified by flash chromatography over silica gel (100-200 mesh) with 55-60% EtOAc/n-hexane to provide the desired pure product (3.38 g, 90% yield) as off white solid.

¹H NMR (CDCl₃, 400 MHz) δ: 8.53 (d, J=1.6 Hz, 1H), 8.51-8.50 (dd, J1=1.6 Hz, J2=4.8 Hz, 1H), 7.62-7.57 (m, 3H), 7.38 (d; J=8.4 Hz, 2H), 7.26-7.21 (m, 3H), 7.16-7.09 (m, 4H), 6.96 (d, J=8.0 Hz, 1H), 6.90-6.88 (m, 2H), 6.84 (s, 1H), 6.66 (t, J=2.0 Hz, 1H), 4.19 (t, J=6.4 Hz, 2H), 4.13 (s, 2H), 4.00 (s, 2H), 3.88 (s, 3H), 3.08 (t, J=6.4 Hz, 2H), 2.95 (s, 3H), 2.31 (s, 3H).

### Example 58

### N-(3-((4-cyanobenzyl)(4-(3-((tetrahydrofuran-3-yl)methoxy)phenoxy)benzyl)amino)-2-methylphenyl)methanesulfonamide

### Step-1: Preparation of 4-(((4-(3-(allyloxy)phenoxy)benzyl)(2-methyl-3-nitrophenyl)amino)methyl)benzonitrile

To a stirred solution of N-(4-(3-(allyloxy)phenoxy)benzyl)-2-methyl-3-nitroaniline (5 g, 12.80 mmol) and 4-(bromomethyl)benzonitrile in DMF (35 mL) maintained under nitrogen, was added DIPEA (5.54 mL, 32.04 mmol) at room temperature, and the resulting reaction mixture was heated at 90-100° C. for 40 h. Reaction mixture was diluted with EtOAc (40 mL), washed with water, brine and organic extract was dried (Na₂SO₄), filtered, and concentrated under vacuum to afford 6.52 g crude product. Crude product was purified by flash chromatography on silica gel (100-200 mesh) with 12% EtOAc/n-hexane to provide the desired pure product (6.26 g, 96% yield) as yellow oil

¹H NMR (CDCl₃, 400 MHz) δ: 7.59-7.53 (m, 3H), 7.31 (d, J=8.4 Hz, 2H), 7.26-7.16 (m, 2H), 7.15-7.07 (m, 3H), 6.94 (d, J=8.4 Hz, 2H), 6.68-6.66 (dd, J₁=2 Hz, J₂=9.2 Hz, 1H), 6.59-6.56 (m, 2H), 6.03-5.99 (m, 1H), 5.41-5.36 (dd, J=1.6 Hz, J₂=17.2 Hz, 1H), 5.29-5.26 (dd, J₁=1.6 Hz, J₂=10.4 Hz, 1H), 4.50 (d, J=5.2 Hz, 2H), 4.17 (s, 2H), 4.05 (s, 2H), 2.60 (s, 3H);

MS (ESI+) m/z 527.7 (M+Na)⁺.

### Step-2: Preparation of 4-(((4-(3-hydroxyphenoxy)benzyl)(2-methyl-3-nitrophenyl)amino)methyl)benzonitrile

To a stirred solution of 4-(((4-(3-(allyloxy)phenoxy)benzyl)(2-methyl-3-nitrophenyl)amino)methyl)benzonitrile (6.2 g, 12.27 mmol, obtained above) in MeOH (40 mL) maintained under nitrogen, was added tetrakis(triphenylphosphine)palladium (0) (0.14 g, 0.12 mmol), potassium carbonate (5.10 g, 36.83 mmol) at room temperature, and the resulting reaction mixture was heated at 50-60° C. for 3 h. The reaction mixture was placed under reduced pressure to remove the solvent. The residue was diluted with EtOAc (40 mL), washed with water, brine and the organic extract was dried (Na₂SO₄), filtered, and concentrated under vacuum to afford 6.11 g crude product. Crude product was purified by flash chromatography on silica gel (100-200 mesh) with 20% EtOAc/n-hexane to provide the desired pure product (5.13 g, 90% yield) as yellow oil.

¹H NMR (CDCl₃, 400 MHz) δ: 7.59-7.57 (m, 3H), 7.32 (d, J=8.0 Hz, 2H), 7.22-7.16 (m, 2H), 7.12-7.09 (m, 3H), 6.93 (d, J=8.0 Hz, 2H), 6.59-6.55 (m, 2H), 6.46 (t, J=2.0 Hz, 1H), 5.05 (s, 1H), 4.17 (s, 2H), 4.05 (s, 2H), 2.57 (s, 3H);

MS (ESI+) m/z 487.7 (M+Na)⁺.

### Step-3: Preparation of 4-(((2-methyl-3-nitrophenyl)(4-(3-((tetrahydrofuran-3-yl)methoxy)phenoxy)benzyl)amino)methyl)benzonitrile

Mixture of 4-(((4-(3-hydroxyphenoxy)benzyl)(2-methyl-3-nitrophenyl)amino)methyl)benzonitrile (5.1 g, 10.96 mmol, obtained in step 2 above), cesium carbonate (10.72 g, 32.90 mmol). (tetrahydrofuran-3-yl)methyl methanesulfonate (2.37 g, 13.16 mmol) in DMF (40 mL) was stirred at room temperature and to it was added catalytic TBAB (50 mg). The resulting reaction mixture was heated at 100-110° C. for 2 h. Reaction mixture was diluted with EtOAc (50 mL), washed with water, brine and the organic extract was dried (Na₂SO₄), filtered, and concentrated under vacuum to afford 5.32 g crude product. Crude product was purified by flash chromatography on silica gel (100-200 mesh) with 22% EtOAc/n-hexane to provide the desired pure product (4.9 g, 90% yield) as yellow oil.

¹H NMR (CDCl₃, 400 MHz) δ: 7.59-7.53 (m, 3H), 7.31 (d, J=8.0 Hz, 2H), 7.25-7.16 (m, 2H), 7.12-7.08 (m, 3H), 6.93 (d, J=8.4 Hz, 2H), 6.66-6.64 (dd, J₁=2 Hz, J₂=8.4 Hz, 1H), 6.57-6.54 (m, 2H), 4.17 (s, 2H), 4.05 (s, 2H), 3.91-3.83 (m, 3H), 3.81-3.74 (m, 2H), 3.70-3.67 (m, 1H), 2.75-2.68 (m, 1H), 2.59 (s, 3H), 2.14-2.04 (m, 1H), 1.75-1.67 (m, 1H);

MS (ESI+) m/z 571.9 (M+Na)⁺.

### Step-4: Preparation of 4-(((3-amino-2-methylphenyl)(4-(3-((tetrahydrofuran-3-yl)methoxy)phenoxy)benzyl)amino)methyl)benzonitrile

Solution of 4-(((2-methyl-3-nitrophenyl)(4-(3-((tetrahydrofuran-3-yl)methoxy)phenoxy)-benzyl)amino)methyl)benzonitrile (3.9 g, 7.12 mmol, from above) and iron metal powder (2.78 g, 49.87 mmol) in EtOH (40 mL) was stirred at room temperature. Ammonium chloride (0.27 g, 4.98 mmol) dissolved in water (5 mL) was added, and the resulting reaction mixture was heated to reflux for 2 h. Reaction mixture was diluted with dichloromethane (50 mL), filtered through celite, residue was washed with dichloromethane, and combined organic extract was dried (Na₂SO₄), filtered, and concentrated under vacuum to provide the desired product (2.58 g, 70% yield) as light brown colored oil.

¹H NMR (CDCl₃, 400 MHz) δ: 7.53 (d, J=8.0 Hz, 2H), 7.31 (d, J=8.0 Hz, 2H), 7.25-7.17 (m, 2H), 6.94-6.87 (m, 3H), 6.64-6.62 (dd, J₁=8.4 Hz, J₂=2.0 Hz, 1H), 6.58-6.54 (m, 2H), 6.47-6.41 (m, 2H), 4.09 (s, 2H), 3.98 (s, 2H), 3.91-3.85 (m, 4H), 3.83-3.48 (m, 4H), 2.74-2.67 (m, 1H), 2.24 (s, 3H), 2.15-2.05 (m, 1H), 1.75-1.67 (m, 1H);

MS (ESI+) m/z 541.7 (M+Na)⁺.

### Step-5: N-(3-((4-cyanobenzyl)(4-(3-((tetrahydrofuran-3-yl)methoxy)phenoxy)benzyl)amino)-2-methylphenyl)methanesulfonamide

To a stirred solution of 4-(((3-amino-2-methylphenyl)(4-(3-((tetrahydrofuran-3-yl)methoxy)phenoxy)benzyl)amino)methyl)benzonitrile (2.54 mL, 4.93 mmol) in pyridine (20 mL) maintained under nitrogen, was added methanesulfonyl chloride (0.48 mL, 5.89 mmol) drop wise at 0-5° C., and the resulting reaction mixture was stirred at room temperature for 2 h. Reaction mixture was diluted with EtOAc (40 mL), washed with water, brine and organic extract was dried (Na₂SO₄), filtered, and concentrated under vacuum to afford 2.73 g crude product. Crude product was purified by flash chromatography on silica gel (100-200 mesh) with 40% EtOAc/n-hexane to provide the desired pure product (2.70 g, 92% yield) as brownish solid.

¹H NMR (CDCl₃, 400 MHz) δ: 7.55 (d, J=8.4 Hz, 3H), 7.32 (d, J=8.0 Hz, 2H), 7.23-7.19 (m, 2H), 7.17-7.09 (m, 3H), 6.92 (d, J=8.8 Hz, 2H), 6.86 (d, J=8.0 Hz, 1H), 6.65-6.63 (dd, J1=8.4 Hz, J2=2.0 Hz, 1H), 6.57-6.55 (dd, J1=8.0 Hz, J2=1.6 Hz, 1H), 6.52 (t, J=2.4 Hz, 1H), 6.20 (s, 1H), 4.12 (s, 2H), 3.99 (s, 2H), 3.91-3.73 (m, 4H), 3.70-3.66 (m, 1H), 2.99 (s, 3H), 2.74-2.68 (m, 1H), 2.37 (s, 3H), 2.13-2.04 (m, 1H), 1.75-1.72 (m, 1H);

MS (ESI+) m/z 620.0 (M+Na)⁺.

### Example 75

### 3-(4-(((4-Cyanobenzyl)(2-methyl-3-(methylsulfonamido)phenyl)amino)methyl)phenoxy)-5-(2-(pyridin-3-yl)ethoxy)benzamide

To a solution of 3-(4-(((4-Cyanobenzyl)(2-methyl-3-(methylsulfonamido)phenyl)amino)methyl)phenoxy)-5-(2-(pyridin-3-yl)ethoxy)benzoic acid (150 mg, 0.22 mmol) in dichloromethane (20 mL) was added EDAC.HCl (87 mg, 0.45 mmol), and HOBt (60 mg, 0.45 mmol) at 25-30° C. and the mixture was stirred at same temperature for 5 minutes and ammonia gas was passed through the reaction mixture for 3 minutes. Reaction was continued under stirring at 25-30° C. for 1 h. After completion of reaction mixture was diluted with dichloromethane (40 mL) and the organic layer was washed with water, brine, dried over Na₂SO₄, filtered and concentrated under reduced pressure to get 130 mg of crude product. Crude product was purified by flash chromatography over silica gel (100-200 mesh) with 4-5% MeOH/CHCl₃ to provide the desired pure product (120 mg, 91% yield) as off white solid.

¹H NMR (CDCl₃, 400 MHz) δ: 8.53 (d, J=1.6 Hz, 1H), 8.51-8.50 (dd, J₁=1.2 Hz, J₂=4.8 Hz, 1H), 7.61-7.58 (m, 3H), 7.42 (d, J=8.0 Hz, 2H), 7.26-7.22 (m, 2H), 7.17-7.14 (m, 2H), 7.08-7.03 (m, 3H), 6.96-6.95 (m, 1H), 6.88-6.86 (m, 2H), 6.76-6.75 (m, 1H), 6.64 (t, J=2.4 Hz, 1H), 6.01 (bs, 1H), 5.70 (bs, 1H), 4.19 (t, J=6.4 Hz, 2H), 4.15 (s, 2H), 3.98 (s, 2H), 3.09 (t, J=6.4 Hz, 2H), 2.94 (s, 3H), 2.25 (s, 3H).

### Example 78

### N-(3-(benzyl(4-(3-(2-(pyridin-3-yl)ethoxy)phenoxy)benzyl)amino)-2-methylphenyl)methanesulfonamide

### Step-1: N-(3-((4-(3-(allyloxy)phenoxy)benzyl)(benzyl)amino)-2-methylphenyl)-N-(methylsulfonyl)methanesulfonamide

To a solution of N1-(4-(3-(allyloxy)phenoxy)benzyl)-N1-benzyl-2-methylbenzene-1,3-diamine (4.2 g, 9.32 mmol) and triethylamine (5.2 ml, 37.3 mmol) in dry DCM (100 ml) mesyl chloride was added (1.52 ml, 19.6 mmol). The reaction mixture was stirred at 40° C. for 1 hour. Organic solvents were evaporated in vacuo and the remaining crude product was purified on SiO₂ (EtOAc/Heptane 0:1-EtOAc/Heptane/acetic acid 1:1:0.1) affording 5.15 g of pure compound (91% yield). ES/MS m/z: 607.2 (M+H); ¹H NMR (CDCl₃, 500 MHz) δ: 7.31-7.19 (m, 6H), 7.16-7.11 (m, 3H), 7.05-7.00 (m, 2H), 6.93 (m, 2H), 6.66 (m, 1H), 6.59-6.56 (m, 2H), 6.03 (m, 1H), 5.39 (m, 1H), 5.28 (m, 1H), 4.50 (m, 2H), 4.08 (s, 2H), 4.03 (s, 2H), 3.44 (s, 6H) and 2.50 (s, 3H).

### Step-2: N-(3-(benzyl(4-(3-hydroxyphenoxy)benzyl)amino)-2-methylphenyl)-N-(methylsulfonyl)methanesulfonamide

To a solution of N-(3-((4-(3-(allyloxy)phenoxy)benzyl)(benzyl)amino)-2-methylphenyl)-N-(methylsulfonyl)methanesulfonamide (5.08 g, 8.36 mmol) and Pd(PPh₃)₄ (0.48 g, 0.42 mmol) in dry DCM (60 ml) phenylsilane was added (2.06 ml, 16.73 mmol) and the resulting mixture was stirred for 2 hours at room temperature. Solvent was evaporated in vacuo and the product was isolated by SiO₂ flash column chromatography (EtOAc/Heptane 0:1-1:1) affording 4.5 g of pure compound (95% yield).

ES/MS m/z: 567.2 (M+H), 565.2 (M−H); ¹H NMR (CDCl₃, 500 MHz) δ: 7.32-7.23 (m, 5H), 7.18-7.10 (m, 5H), 7.03 (m, 1H), 6.91 (m, 2H), 6.57 (m, 1H), 6.53 (m, 1H), 6.29 (t, 1H, J=2.4 Hz), 4.07 (s, 2H), 4.03 (s, 2H), 3.43 (s, 6H) and 2.43 (s, 3H).

### Step-3: N-(3-(benzyl(4-(3-(2-(pyridin-3-yl)ethoxy)phenoxy)benzyl)amino)-2-methyl phenyl)methanesulfonamide

N-(3-(benzyl(4-(3-hydroxyphenoxy)benzyl)amino)-2-methylphenyl)-N-(methylsulfonyl)methanesulfonamide (15 mg, 0.03 mmol), 2-(pyridin-3-yl)ethanol (8.15 mg, 0.07 mmol) and PPh₃ (10.4 mg, 0.04 mmol) were dissolved in 0.6 ml of dry THF and resulting solution was cooled down to 0° C. under nitrogen atmosphere. DBAD (9.14 mg, 0.04 mmol) dissolved in 0.2 ml of dry THF was added. The reaction mixture was stirred at room temperature over night. Organic solvents were removed and the residue was redisolved in 1 ml of DMF/H₂O (6:1). K₂CO₃ (18.3 mg, 0.13 mmol) was added and the resulting mixture was stirred at 80° C. for 2 hours. Evaporation of solvents and purification on preparative C8 HPLC using acidic (formic acid, pH2.8) CH₃CN/H₂O gradient (35-65% MeCN over 40 min) afforded 8 mg of pure desired product (51% yield).

ES/MS m/z: 594.4 (M+H), 592.3 (M−H); ¹H NMR (CDCl₃, 500 MHz) δ: 8.54-8.49 (m, 2H), 7.60 (m, 1H), 7.29-7.26 (m, 2H), 7.25-7.18 (m, 5H), 7.15-7.10 (m, 3H), 6.93 (dd, J₁=0.8, J₂=8.1 Hz), 6.89 (m, 2H), 6.62-6.55 (m, 3H), 6.46 (t, 1H, J=2.3 Hz), 4.13 (t, 2H, J=6.6 Hz), 4.05 (s, 2H9, 4.02 (s, 2H), 3.06 (t, 2H, J=6.6 Hz), 2.94 (s, 3H) and 2.35 (s, 3H).

### Example 84

### N-(3-((4-(3-cyano-5-(2-(pyridin-3-yl)ethoxy)phenoxy)benzyl)(4-cyanobenzyl)amino)-2-methylphenyl)methanesulfonamide

Into the solution of 3-(4-(((4-Cyanobenzyl)(2-methyl-3-(methylsulfonamido)phenyl)amino)methyl)phenoxy)-5-(2-(pyridin-3-yl)ethoxy)benzamide (70 mg, 0.10 mmol) in DMF (2.0 mL) was added P₂O₅ (150 mg, 1.05 mmol) at once. The mixture was heated in the microwave oven at 800 W for 5 minutes. After the completion of reaction the reaction mixture was quenched with ice water and compound was extracted in EtOAc. Combined EtOAc layer was washed with water, brine, dried over Na₂SO₄, filtered and evaporated under reduced pressure to get 14 mg of desired compound. Compound was purified by preparative TLC with 2% MeOH/CHCl₃ to provide the desired pure product (14 mg, 20% yield) as off white solid.

¹H NMR (CDCl₃, 400 MHz) δ: 8.59-8.51 (m, 2H), 7.60-7.57 (m, 3H), 7.35 (d, J=8.0 Hz, 2H), 7.32-7.25 (m, 1H), 7.20-7.17 (m, 3H), 7.12 (t, J=8.0 Hz, 1H), 6.92-6.87 (m, 3H), 6.83-6.82 (m, 1H), 6.78-6.77 (m, 1H), 6.64-6.63 (m, 2H), 4.16-4.13 (m, 4H), 4.04 (s, 2H), 3.08 (t, J=6.0 Hz, 2H), 3.00 (s, 3H), 2.39 (s, 3H).

### Example 97

### N1-(4-(4-(((4-cyanobenzyl)(2-methyl-3-(methylsulfonamido)phenyl)amino)methyl)-phenoxy)-2-(2-(pyridin-3-yl)ethoxy)phenyl)-N3-methylmalonamide

### Step-1: N-(3-((4-(allyloxy)benzyl)(4-cyanobenzyl)amino)-2-methylphenyl)-N-(methylsulfonyl)methanesulfonamide

A solution of N-(3-((4-(allyloxy)benzyl)(4-cyanobenzyl)amino)-2-methylphenyl)methanesulfonamide (3124 mg, 6.8 mmol), triethylamine (4150 μL, 4.4 eq) and mesyl chloride (1362 μL, 2.6 eq) in dry DCM (50 mL) was stirred at 40° C. over night. Reaction was quenched with water and product extracted with DCM. The organic solvents were evaporated and the remaining residue was purified on silica column (EtOAc/Hexane 0:1-1:1) affording 3.6 g of pure product as a white sticky solid (98% yield).

¹H NMR (MeOD, 500 MHz): 7.57 (m, 2H), 7.38 (m, 2H), 7.14-7.05 (m, 5H), 6.81 (m, 2H), 6.03 (m, 1H), 5.36 (m, 1H), 5.22 (m, 1H), 4.49 (m, 2H), 4.15 (s, 2H), 4.00 (s, 2H), 3.43 (s, 6H) and 2.46 (s, 3H);

MS (ESI) m/z 540.2 (M+H), 538.5 (M−H).

### Step-2: N-(3-((4-cyanobenzyl)(4-hydroxybenzyl)amino)-2-methylphenyl)-N-(methylsulfonyl)methanesulfonamide

N-(3-((4-(allyloxy)benzyl)(4-cyanobenzyl)amino)-2-methylphenyl)-N-(methylsulfonyl)methanesulfonamide (1.85 g, 3.4 mmol) and Pd(PPh₃)₄ (198 mg, 0.05 eq.) were dissolved in DCM (60 mL). Nitrogen gas was bubbled through the solution, followed by addition of phenylsilane (846 μL, 2 eq). The reaction mixture was stirred for 4 hours at room temperature under argon atmosphere. Organic solvents were evaporated and the crude residue was purified on a silica column (EtOAc/Heptane 0:1-1:1) giving 1604 mg of required compound as a white solid (94% yield).

¹H NMR (MeOD, 500 MHz): 7.57 (m, 2H), 7.38 (m, 2H), 7.14-7.05 (m, 3H), 6.98 (m, 2H), 6.67 (m, 2H), 4.15 (s, 2H), 3.96 (s, 2H), 3.43 (s, 6H) and 2.45 (s, 3H); MS (ESI) m/z 500.1 (M+H), 498.3 (M−H).

### Step-3: N-(3-((4-cyanobenzyl)(4-(4-nitro-3-(2-(pyridin-3-yl)ethoxy)phenoxy)benzyl)amino)-2-methylphenyl)-N-(methylsulfonyl)methanesulfonamide

To a dried flask containing 6 g of crushed molecular sieves was added Cu(OAc)₂ (671 mg, 1.2 eq), N-(3-((4-cyanobenzyl)(4-hydroxybenzyl)amino)-2-methylphenyl)-N-(methylsulfonyl)methanesulfonamide (1.54 g, 3.1 mmol) dissolved in DCM (50 mL), 4-nitro-3-(2-(pyridin-3-yl)ethoxy)phenylboronic acid (1.77 g, 2 eq) dissolved in DCM (50 mL). Pyridine (1245 μL, 5 eq) and triethylamine (2142 μL, 5 eq) were added and the reaction mixture was stirred at ambient temperature and atmosphere for 2 days. Addition of water and DCM, extraction followed by solvents evaporation gave brown oil. Product was purified on a silica column (Heptane/EtOAc 1:0-0:1) giving 950 mg of the title compound as a slightly sticky oil, (42% yield).

¹H NMR (acetone-d6, 500 MHz): 8.57 (d, 1H, J=2.0 Hz), 8.43 (dd, 1H, J=4.8, 1.6 Hz), 7.91 (d, 1H, J=9.0 Hz), 7.77 (m, 1H), 7.69 (m, 2H), 7.53 (m, 2H), 7.35 (m, 2H), 7.28 (m, 1H), 7.22-7.14 (m, 3H), 7.05 (m, 2H), 6.85 (d, 1H, J=2.5 Hz), 6.52 (dd, 1H, J=9.0, 2.5 Hz), 4.36 (t, 211, J=6.5 Hz), 4.28 (s, 2H), 4.18 (s, 2H), 3.50 (s, 6H, 3.15 (t, 2H, J=6.5 Hz) and 2.54 (s, 3H);

MS (ESI) m/z 742.3 (M+H)

### Step-4: N-(3-((4-(4-amino-3-(2-(pyridin-3-yl)ethoxy)phenoxy)benzyl)(4-cyanobenzyl)amino)-2-methylphenyl)-N-(methylsulfonyl)methanesulfonamide

N-(3-((4-cyanobenzyl)(4-(4-nitro-3-(2-(pyridin-3-yl)ethoxy)phenoxy)benzyl)amino)-2-methylphenyl)-N-(methylsulfonyl)methanesulfonamide (450 mg, 0.6 mmol) was dissolved in a mixture of EtOAc and EtOH (3:5) and the resulting solution was added to a vial containing Fe (475 mg, 14 eq) and NH₄Cl (45 mg, 1.4 eq dissolved in 0.5 mL of H₂O). The vial was sealed and heated at 75° C. for 1 hour. The mixture was cooled and diluted with DCM (30 mL), followed by filtration through Celite. Filtrate was concentrated, water was added and product extracted with DCM. Solvent evaporation afforded N-(3-((4-(4-amino-3-(2-(pyridin-3-yl)ethoxy)phenoxy)benzyl)(4-cyanobenzyl)amino)-2-methylphenyl)-N-(methylsulfonyl)methanesulfonamide (414 mg, 96%) as an light brown oil. Compound was utilised in the next step without further purification.

MS (ESI) m/z 712.3 (M+H)

### Step-5: Methyl 3-(4-(4-(((4-cyanobenzyl)(2-methyl-3-(N-(methylsulfonyl)methylsulfonamido)phenyl)amino)methyl)phenoxy)-2-(2-(pyridin-3-yl)ethoxy)phenylamino)-3-oxopropanoate

Solution of N-(3-((4-(4-amino-3-(2-(pyridin-3-yl)ethoxy)phenoxy)benzyl)(4-cyanobenzyl)amino)-2-methylphenyl)-N-(methylsulfonyl)methanesulfonamide (42 mg, 0.06 mmol) and triethylamine (10 μL, 0.07 mmol) in DCM (2 mL) was cooled down to 0° C. Methyl 3-chloro-3-oxopropanoate (7 μL, 0.06 mmol) was added. The reaction mixture was stirred at 0° C. for 2 hours and then at room temperature overnight. Saturated aqueous NH₄Cl was added and product extracted with DCM. Resulting crude material (48 mg, quantitative yield) was used in the next step without further purification.

MS (ESI) m/z 812.3 (M+H), 810.3 (M−H).

### Step-6: 3-(4-(4-(((4-cyanobenzyl)(2-methyl-3-(N-(methylsulfonyl)methylsulfonamido)phenyl)amino)methyl)phenoxy)-2-(2-(pyridin-3-yl)ethoxy)phenylamino)-3-oxopropanoic acid

Methyl 3-(4-(4-(((4-cyanobenzyl)(2-methyl-3-(N-(methylsulfonyl)methylsulfonamido)phenyl)amino)methyl)phenoxy)-2-(2-(pyridin-3-yl)ethoxy)phenylamino)-3-oxopropanoate (48 mg, 0.06 mmol) was dissolved in THF (2 mL) and 0.5 mL of 1M aqueous LiOH were added. The reaction mixture was stirred at room temperature for 3 hours. The reaction mixture was titrated with 1M aqueous HCl until pH 2 followed by product extraction with DCM. Resulting compound (41 mg, 96% yield) was used in the next step without further purification.

¹H NMR (CDCl₃, 500 MHz): 8.84 (br s, 1H), 8.44 (br s, 1H), 8.23 (d, 1H, J=8.7 Hz), 7.73 (d, 1H, J=7.8 Hz), 7.55 (m, 2H), 7.38-7.30 (m, 3H), 7.18 (d, 1H, J=7.9 Hz), 7.12-7.06 (m, 3H), 6.84 (m, 2H), 6.56-6.50 (m, 2H), 4.17 (t, 2H, J=5.7 Hz), 4.11 (s, 2H), 3.98 (s, 2H), 3.50 (br s, 2H), 3.17 (t, 2H, J=5.7 Hz), 2.99 (s, 3H) and 2.36 (s, 3H);

MS (ESI) m/z 720.3 (M+H), 718.4 (M−H)

### Step-7: N1-(4-(((4-(4-cyanobenzyl)(2-methyl-3-(methylsulfonamido)phenyl)amino)methyl)phenoxy)-2-(2-(pyridin-3-yl)ethoxy)phenyl)-N3-methylmalonamide

3-(4-(4-(((4-cyanobenzyl)(2-methyl-3-(N-(methylsulfonyl)methylsulfonamido)phenyl)amino)methyl)phenoxy)-2-(2-(pyridin-3-yl)ethoxy)phenylamino)-3-oxopropanoic acid (15 mg, 0.02 mmol) was dissolved in DMF (1 mL) followed by addition of N-ethyl-N-isopropylpropan-2-amine (8 μL, 0.05 mmol) and O-(1H-benzotriazol-1-yl)-N,N,N′,N′-tetramethyluronium tetrafluoroborate (7.4 mg, 0.02 mmol). Methylamine was added as 2M THF solution (15 μL, 0.03 mmol) and the reaction mixture was stirred overnight at room temperature. Saturated aqueous NH₄Cl was added and product extracted with DCM. Residue obtained after evaporation of organic solvents was separated by preparative C8 HPLC using acidic (0.05% HCOOH, pH2.8) CH₃CN/H₂O gradient (40-70% CH₃CN over 15 minutes) to afford 4 mg of pure product (26% yield).

¹H NMR (CDCl₃, 500 MHz): 8.68 (br s, 1H), 8.52 (d, 1H, J=4.3 Hz), 8.15 (d, 1H, J=8.7 Hz), 7.83 (m, 1H), 7.55 (m, 2H), 7.37-7.30 (m, 3H), 7.18 (d, 1H, J=8.2 Hz), 7.11-7.08 (m, 3H), 6.87-6.84 (m, 3H), 6.56-6.53 (m, 2H), 4.18 (t, 2H, J=6.1 Hz), 4.10 (s, 2H), 3.98 (s, 2H), 3.35 (s, 2H), 3.21 (t, 2H, J=6.1 Hz), 2.99 (s, 3H), 2.89 (s, 3H) and 2.36 (s, 3H);

MS (ESI) m/z 733.4 (M+H), 731.4 (M−H).

### Example 98

### N-(3-((4-cyanobenzyl)(4-(4′-hydroxybiphenyl-3-yloxy)benzyl)amino)-2-methylphenyl)methanesulfonamide

### Step-1: N-(3-((4-(3-bromophenoxy)benzyl)(4-cyanobenzyl)amino)-2-methylphenyl)methanesulfonamide

A flask was charged with 8.44 g of crushed molecular sieves and copper acetate (1.08 g, 6 mmol). 3-Bromophenylboronic acid (2.4 g, 11.9 mmol), N-(3-((4-cyanobenzyl)(4-hydroxybenzyl)amino)-2-methylphenyl)methanesulfonamide (2.5 g, 5.9 mmol) and pyridine (2.4 mL, 30 mmol) dissolved in dry DCM (50 mL) were added. The reaction mixture was stirred at ambient temperature and atmosphere for 17 hours. Additional amounts of 3-bromophenylboronic acid (1.2 g, 6 mmol) and copper acetate (1.1 g, 6 mmol) were added and stirring continued for another 20 hours. DCM and water were added. The product was extracted with DCM and purified by flash chromatography on silica gel (CH₂Cl₂/isohexane 1:1->CH₂Cl₂/MeOH 39:1) to provide the desired product (1.84 g, 54% yield).

¹H NMR (acetone-d6, 500 MHz): 7.88 (br s, 1H), 7.68 (m, 2H), 7.54 (m, 2H), 7.36 (m, 2H), 7.34-7.36 (m, 2H), 7.18 (d, J=8.15 Hz, 1H), 7.13 (m, 1H), 7.09 (t, J=7.83 Hz, 1H), 7.04 (d, J=8.30 Hz, 1H), 7.01-6.90 (m, 3H), 4.26 (s, 2H), 4.14 (s, 2H), 2.94 (s, 3H), 2.53 (s, 3H);

MS (ESI) m/z 576.2/578.1 (M+H), 574.1/576.2 (M−H).

### Step-2: N-(3-((4-cyanobenzyl)(4-(4′-hydroxybiphenyl-3-yloxy)benzyl)amino)-2-methylphenyl)methanesulfonamide

N-(3-((4-(3-bromophenoxy)benzyl)(4-cyanobenzyl)amino)-2-methylphenyl)methanesulfonamide (30 mg, 0.05 mmol), 4-(4,4,5,5-tetramethyl-1,3,2-dioxaborolan-2-yl)phenyl acetate (20 mg, 0.08 mmol), S-Phos (2 mg, 0.01 mmol), palladium acetate (0.3 mg, 0.001 mmol) and potassium carbonate (14 mg, 0.1 mmol) were dissolved in 1 mL of toluene/EtOH/H₂O 100:10:1 solvent mixture. Reaction mixture was stirred at 60° C. under nitrogen atmosphere for 3 hours. Water was added and product extracted with DCM. Crude material was purified on a silica column (DCM/MeOH 99:1) and further purified by preparative HPLC (40-90% MeOH/0.05% HCOOH, pH2.8, 25 mL/min, 30 minute gradient time). Yield 16 mg (52%).

1H (acetone-d6, 500 MHz): 7.67 (m, 2H), 7.53 (m, 2H), 7.48 (m, 2H), 7.40 (t, 1H, J=8.1 Hz), 7.35 (m, 1H), 7.32 (m, 2H), 7.19-7.16 (m, 2H), 7.08 (t, 1H, J=8.2 Hz), 7.03 (dd, 1H, J=8.1, 1.0 Hz), 6.97 (m, 2H), 6.91 (m, 2H), 6.88 (m, 1H), 4.25 (s, 2H9, 4.12 (s, 2H), 2.93 (s, 3H9 and 2.53 (s, 3H);

MS (ESI) m/z 590.3 (M+H), 588.3 (M−H).

### Example 101

### N-(3-((4-cyanobenzyl)(4-(4-(2-(pyridin-3-yl)ethoxy)pyridin-2-yloxy)benzyl)amino)-2-methylphenyl)methanesulfonamide

The title compound was made from N-(3-((4-cyanobenzyl)(4-hydroxybenzyl)amino)-2-methylphenyl)-N-(methylsulfonyl)methanesulfonamide (8 mg, 0.02 mmol) 2-(2-(pyridin-3-yl)ethoxy)pyridin-4-ylboronic acid (8 mg, 0.03 mmol) triethylamine and copper acetate utilizing the same procedure as described for N-(3-((4-(3-bromophenoxy)benzyl)(4-cyanobenzyl)amino)-2-methylphenyl)methanesulfonamide (see preparation of Example 98, step 1). After all the 2-(2-(pyridin-3-yl)ethoxy)pyridin-4-ylboronic acid was consumed CHCl₃ and water were added. The organic phase was evaporated and the residue was treated with THF and NaOH (1M aq) at 50° C. for 1 h. CHCl₃ and water were added and the mixture was filtered through a phase separator. The organic phase was evaporated and the residue subjected to preparative HPLC (0.05% aqueous HCOOH, 30-100% MeOH, in 30 to minutes, 25 mL/min) to give 2 mg of the desired product (18% yield).

1H-NMR (acetone-d6, 500 MHz): 8.51 (d, 1H, J=1.7 Hz), 8.43 (dd, 1H, J=4.9, 1.7 Hz), 8.01 (d, 1H, J=6.0 Hz), 7.72-7.68 (m, 3H), 7.56 (m, 2H), 7.41 (m, 2H), 7.28 (m, 1H), 7.19 (dd, 1H, J=7.8, 1.3 Hz), 7.10 (t, 1H, J=7.8 Hz), 7.07-7.04 (m, 3H), 6.54 (dd, 1H, J=5.9, 2.2 Hz), 6.02 (d, 1H, J=2.2 Hz), 4.51 (t, 2H, J=6.8 Hz), 4.28 (s, 2H), 4.18 (s, 2H), 3.06 (t, 1H, J=6.8 Hz), 2.94 (s, 3H) and 2.53 (s, 3H);

MS (ESI) m/z 620.2 (M+H), 618.3 (M−H).

### Example 105

### N-(3-((4-cyanobenzyl)(4-(3-(2-(pyridin-3-yl)ethyl)phenoxy)benzyl)amino)-2-methylphenyl)methanesulfonamide

### Step-1: (E)-N-(3-((4-cyanobenzyl)(4-(3-(2-(pyridin-3-yl)vinyl)phenoxy)benzyl)amino)-2-methylphenyl)methanesulfonamide

N-(3-((4-(3-bromophenoxy)benzyl)(4-cyanobenzyl)amino)-2-methylphenyl)methanesulfonamide (20 mg, 0.03 mmol), 3-vinylpyridine (7 mg, 0.07 mmol), palladium acetate (0.8 mg, 0.001 mmol), tri-o-tolylphosphine (1.1 mg, 0.003 mmol), triethylamine (14 μL, 0.1 mmol) and DMF (0.5 mL) were mixed in a microwave vessel which was sealed and purged with N₂. The vial was heated to 130° C. for 15 minutes. Water and CH₂Cl₂ were added, the mixture was filtered through a phase separator, the organic phase was evaporated and the residue was purified by preparative HPLC using 30-75% AcCN/0.05% aqueous HCOOH, (pH2.8), 25 mL/min, 25 minutes gradient. Yield 3 mg (14%).

1H-NMR (acetone-d6, 500 MHz): 8.76 (d, 1H, J=1.6 Hz), 8.46 (m, 1H), 8.00 (m, 1H), 7.68 (m, 2H), 7.54 (m, 2H), 7.42-7.26 (m, 8H), 7.17 (d, 1H, J=7.6 Hz), 7.11-7.04 (m, 2H), 6.96 (m, 2H), 6.91 (m, 1H), 4.26 (s, 2H), 4.14 (s, 2H), 2.94 (s, 3H) and 2.53 (s, 3H);

MS (ESI) m/z 601.2 (M+H), 599.2 (M−H).

### Step-2: N-(3-((4-cyanobenzyl)(4-(3-(2-(pyridin-3-yl)ethyl)phenoxy)benzyl)amino)-2-methylphenyl)methanesulfonamide

A mixture of (E)-N-(3-((4-cyanobenzyl)(4-(3-(2-(pyridin-3-yl)vinyl)phenoxy)benzyl)amino)-2-methylphenyl)methanesulfonamide (2.5 mg, 0.004 mmol) and 0.5 mg of 5% palladium on carbon in 1 mL of EtOH was stirred under 3 PSI of H₂-gas for 1 h. The mixture was filtered through celite and the filtrate was evaporated. The product was purified by preparative HPLC (0.05% aqueous HCOOH, pH2.8, 20-100% AcCN in 25 minutes, 25 mL/min). Yield 1.4 mg (56%).

1H-NMR (acetone-d6, 500 MHz): 8.39-8.37 (m, 2H), 7.68 (m, 2H), 7.58-7.53 (m, 3H), 7.30-7.26 (m, 3H), 7.23 (m, 1H), 7.17 (dd, 1H, J=7.9, 1.2 Hz), 7.09 (t, 1H, J=7.8 Hz), 7.05-7.00 (m, 2H), 6.84 (m, 2H), 6.81-6.79 (m, 2H), 4.26 (s, 2H), 4.12 (s, 2H), 2.94 (s, 3H), 2.93 (s, 4H) and 2.53 (s, 3H);

MS (ESI) m/z 603.2 (M+H), 601.2 (M−H).

### Example 106

### N-(3-((4-cyanobenzyl)(4-(3-((pyridin-3-ylmethoxy)methyl)phenoxy)benzyl)amino)-2-methylphenyl)methanesulfonamide

### Step-1: N-(3-((4-cyanobenzyl)(4-(3-formylphenoxy)benzyl)amino)-2-methylphenyl)-N-(methylsulfonyl)methanesulfonamide

The title compound was made from N-(3-((4-cyanobenzyl)(4-hydroxybenzyl)amino)-2-methylphenyl)-N-(methylsulfonyl)methanesulfonamide (360 mg, 0.72 mmol) using 3-formylphenylboronic acid, triethylamine and copper acetate utilizing the same procedure as described for N-(3-((4-(3-bromophenoxy)benzyl)(4-cyanobenzyl)amino)-2-methylphenyl)methanesulfonamide (see preparation of Example 98, step 1). Yield 394 mg (91%).

¹H NMR (acetone-d6, 500 MHz): 10.00 (s, 1H), 7.70-7.65 (m, 3H), 7.60 (t, J=7.93 Hz, 1H), 7.52 (m, 2H), 7.45 (m, 1H), 7.34-7.29 (m, 3H), 7.21-7.16 (m, 3H), 6.99 (m, 2H), 4.27 (s, 2H), 4.15 (s, 2H), 3.50 (s, 6H), 2.56 (s, 3H);

MS (ESI) m/z 604.1 (M+H), 602.4 (M−H).

### Step-2: N-(3-((4-cyanobenzyl)(4-(3-(hydroxymethyl)phenoxy)benzyl)amino)-2-methylphenyl)-N-(methylsulfonyl)methanesulfonamide

N-(3-((4-cyanobenzyl)(4-(3-formylphenoxy)benzyl)amino)-2-methylphenyl)-N-(methylsulfonyl)methanesulfonamide (214 mg, 0.35 mmol) was dissolved MeOH/THF 1:1 mixture (4 mL) followed by addition of acetic acid (405 μL, 0.71 mmol). NaBH₄ (54 mg, 1.42 mmol) was added portion wise and the reaction mixture was stirred at room temperature for 3 hours. Water was added and product extracted with DCM. Silica column chromatography (CH₂Cl₂/MeOH 39:1) afforded 164 mg of pure product (76% yield).

¹H NMR (acetone-d6, 500 MHz): 7.67 (m, 2H), 7.50 (m, 2H), 7.32 (t, J=7.86 Hz, 1H), 7.26 (m, 2H), 7.19-7.14 (m, 3H), 7.11 (m, 1H), 7.03 (m, 1H), 6.91 (m, 2H), 6.85 (m, 1H), 4.63 (d, J=5.82 Hz, 2H), 4.25 (s, 2H), 4.12 (s, 2H), 3.50 (s, 6H), 2.56 (s, 3H);

MS (ESI) m/z 606.1 (M+H), 604.4 (M−H)

### Step-3: 3-(4-(((4-cyanobenzyl)(2-methyl-3-(N-(methylsulfonyl)methylsulfonamido)phenyl)amino)methyl)phenoxy)benzyl methanesulfonate

N-(3-((4-cyanobenzyl)(4-(3-(hydroxymethyl)phenoxy)benzyl)amino)-2-methylphenyl)-N-(methylsulfonyl)methanesulfonamide (48 mg, 0.08 mmol) was dissolved in 1 mL of dry DCM and triethylamine (18 μL, 0.13 mmol) was added. To resulting solution methanesulfonyl chloride (8 μL, 0.1 mmol) was added and the reaction mixture was stirred at room temperature for 30 minutes. Water was added and product extracted with DCM. Product was used in the next step without further purification. Yield 55 mg (quantitative).

¹H NMR (acetone-d6, 500 MHz): 7.68 (m, 2H), 7.51 (m, 2H), 7.43 (t, J=7.89 Hz, 1H), 7.28 (m, 2H), 7.23 (m, 1H), 7.21-7.15 (m, 3H), 7.11 (m, 1H), 7.00 (m, 1H), 6.94 (m, 2H), 5.61 (s, 2H), 4.26 (s, 2H), 4.13 (s, 2H), 3.50 (s, 6H), 3.11 (s, 3H), 2.55 (s, 3H);

MS (ESI) m/z 684.1 (M+H)

### Step-4: N-(3-((4-cyanobenzyl)(4-(3-((pyridin-3-ylmethoxy)methyl)phenoxy)benzyl)amino)-2-methylphenyl)methanesulfonamide

Pyridin-3-ylmethanol (7 μL, 0.075 mmol) and was dissolved in dry DMF (0.3 mL) and sodium hydride (2 mg, 0.07 mmol) was added. The reaction mixture was stirred at room temperature for 15 minutes. 3-(4-(((4-cyanobenzyl)(2-methyl-3-(N-(methylsulfonyl)methylsulfonamido)phenyl)amino)methyl)phenoxy)benzyl methanesulfonate (10 mg, 0.015 mmol) dissolved in 0.2 mL of dry DMF was added and the reaction mixture was stirred at room temperature for 30 minutes. Saturated aqueous NH₄Cl was added and product extracted with DCM. Silica column chromatography (DCM/MeOH 39:1) followed by preparative HPLC (0.05% aqueous HCOOH, 50-70% AcCN in 25 min, 25 mL/min) gave 4 mg of the desired product (44% yield).

1H-NMR (acetone-d6, 500 MHz): 8.56 (s, 1H), 8.50 (dd, 1H, J=4.7, 1.1 Hz), 7.75 (m, 1H), 7.67 (m, 2H), 7.54 (m, 2H), 7.38-7.30 (m, 4H), 7.18-7.14 (m, 2H), 7.09 (t, 1H, J=8.1 Hz), 7.04-7.02 (m, 2H), 6.94-6.90 (m, 3H), 4.60 (s, 4H), 4.25 (s, 2H), 4.12 (s, 2H), 2.93 (s, 3H) and 2.52 (s, 3H);

MS (ESI) m/z 619.2 (M+H), 617.2 (M−H).

### Example 109

### N-(3-((4-cyano benzyl)(4-(2-(propylthio)-6-(2-(pyridin-3-yl)ethoxy)pyridin-4-yloxy)benzyl)amino)-2-methylphenyl)methanesulfonamide

### Step-1: N-(3-((4-cyanobenzyl)(4-(2,6-difluoropyridin-4-yloxy)benzyl)amino)-2-methylphenyl)methanesulfonamide

To a stirred solution of 2,4,6-trifluoropyridine (30 mg, 0.22 mmol) and N-(3-((4-cyanobenzyl)(4-hydroxybenzyl)amino)-2-methylphenyl)-N-(methylsulfonyl)methanesulfonamide (50 mg, 0.10 mmol) in MeCN (0.5 mL) was added DBU (48 μL, 0.32 mmol) at room temperature, and the resulting reaction mixture was stirred for 20 hours. Aqueous NH₄Cl was added and product extracted with EtOAc (3×2 mL). Organic phase was concentrated under vacuum and crude product was purified by flash chromatography on silica gel (EtOAc/Heptane 0:1-1:1) to provide the desired product (25 mg, 54% yield) as a colourless solid.

¹H NMR (CDCl₃, 500 MHz): 7.57 (m, 2H), 7.33 (m, 2H), 7.28 (m, 2H), 7.20 (m, 2H), 7.12 (m, 1H), 7.03 (m, 2H), 6.86 (d, 1H, J=7.9 Hz), 6.26 (s, 2H), 4.15 (s, 2H), 4.08 (s, 2H), 3.02 (s, 3H) and 2.41 (s, 3H);

MS (ESI) m/z 535.4 (M+H), 533.3 (M−H).

### Step-2: N-(3-((4-cyanobenzyl)(4-(2-fluoro-6-(2-(pyridin-3-yl)ethoxy)pyridin-4-yloxy)benzyl)amino)-2-methylphenyl)methanesulfonamide

To a stirred solution of 2-(pyridin-3-yl)ethanol (6 mg, 0.05 mmol) in MeCN (0.3 mL) was added NaH (0.05 mmol) at 0° C. After 30 minutes stirring at room temperature, a solution of N-(3-(benzyl(4-(2,6-difluoropyridin-4-yloxy)benzyl)amino)-2-methylphenyl)methanesulfonamide (25 mg, 0.05 mmol) in MeCN (0.3 mL) was added. The resulting reaction mixture was stirred at 85° C. for 1 hour. Addition of water, extraction with EtOAc (3×2 mL) and concentration under vacuum gave a crude product. Filtration through silica gel column afforded 19 mg of pure product (54% yield).

¹H NMR (CDCl₃, 500 MHz): 8.54 (br s, 2H), 7.75 (d, 1H, J=8.4 Hz), 7.60 (m, 2H), 7.39 (m, 2H), 7.19 (m, 2H), 7.16-7.12 (m, 2H), 6.97-6.93 (m, 3H), 6.08 (d, 1H, J=1.6 Hz), 5.81 (d, 1H, J=1.6 Hz), 4.47 (t, 2H, J=6.3 Hz), 4.15 (s, 2H), 4.04 (s, 2H), 3.08 (t, 2H, J=6.3 Hz), 3.00 (s, 3H) and 2.37 (s, 3H);

MS (ESI) m/z 638.2 (M+H), 636.2 (M−H).

### Step-3: N-(3-((4-cyanobenzyl)(4-(2-(propylthio)-6-(2-(pyridin-3-yl)ethoxy)pyridin-4-yloxy)benzyl)amino)-2-methylphenyl)methanesulfonamide

To a stirred solution of N-(3-((4-cyanobenzyl)(4-(2-fluoro-6-(2-(pyridin-3-yl)ethoxy)pyridin-4-yloxy)benzyl)amino)-2-methylphenyl)methanesulfonamide (19 mg, 0.03 mmol) in dry DMSO (0.3 mL) propane-1-thiol (5 μL, 0.06 mmol) and potassium carbonate (8 mg, 0.06 mmol) were added. Resulting mixture was stirred at 120° C. for 1 hour. Addition of brine and neutralization with saturated aqueous ammonium chloride solution followed by extraction with EtOAc (3×1 mL) and concentration under vacuum gave crude product which was purified on preparative C8 HPLC using neutral (NH₄OAc, pH6.8) CH₃CN/H₂O gradient (20-95% CH₃CN over 35 minutes, 25 mL/min) to afford 0.75 mg of pure product (4% yield).

¹H NMR (CDCl₃, 500 MHz): 8.59 (br s, 1H), 8.55 (d, 1H, J=4.9 Hz), 7.59 (m, 2H), 7.48 (m, 1H), 7.39 (m, 2H), 7.22 (m, 1H), 7.18-7.12 (m, 3H), 7.05 (m, 1H), 6.96-6.92 (m, 3H), 6.41 (d, 1H, J=1.6 Hz), 5.68 (d, 1H, J=1.9 Hz), 4.54 (t, 2H, J=6.3 Hz), 4.15 (s, 2H), 4.02 (s, 2H), 3.12 (m, 2H), 3.06 (t, 2H, J=6.3 Hz), 3.00 (s, 3H), 2.34 (s, 3H), 1.73 (m, 2H) and 1.02 (t, 3H, J=7.5 Hz);

MS (ESI) m/z 694.2 (M+H), 692.2 (M−H).

The compounds listed below were prepared following analogous procedure as provided in the above detailed examples and/or the general reaction schemes above, with suitable modifications and alterations as necessary.

| Ex- | | | |
|---|---|---|---|
| am- | | | |
| ple | | | |
| No. | Structure | Name | ¹H NMR |
| 1) | 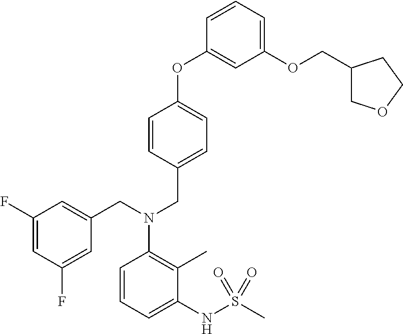 | N-(3-((2,4- difluorobenzyl)(4-(3- ((tetrahydrofuran-3- yl)methoxy)phenoxy) benzyl)amino)-2- methylphenyl) methanesulfonamide | (CDCl₃, 400 MHz) δ 7.30-7.18 (m, 4H), 7.13 (t, J = 8.0 Hz, 2H), 6.93- 6.90 (m, 3H), 6.76-6.69 (m, 2H), 6.64-6.62 (m, 1H), 6.57-6.51 (m, 2H), 6.14 (s, 1H), 4.07 (s, 2H), 4.04 (s, 2H), 3.91-9.85 (m, 4H), 3.83-3.73 (m, 1H), 3.70-3.66 (m, 1H), 2.95 (s, 3H), 2.71 (t, J = 6.8 Hz, 1H), 2.3 (s, 3H), 2.11-2.07 (m, 1H), 1.73-1.56 (m, 1H) m/z (M + Na)⁺: 631.00 |
| 2) | 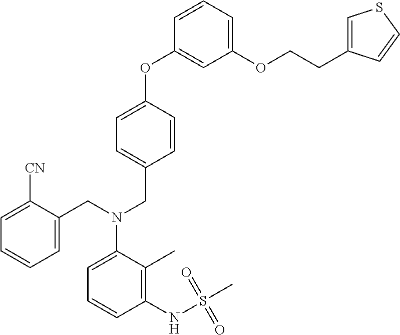 | N-(3-((2- cyanobenzyl)(4-(3- (2-(thiophen-3- yl)ethoxy)phenoxy) benzyl)amino)-2- methylphenyl) methanesulfonamide | (CDCl₃, 400 MHz) δ 7.57 (d, J = 7.6 Hz, 1H), 7.47 (t, J = 7.6 Hz, 1H), 7.41 (d, J = 7.6 Hz, 1H), 7.30- 7.25, (m, 2H), 7.23-7.18 (m, 2H), 7.16-7.13 (m, 3H), 7.06 (d, J = 2.0 Hz, 1H), 7.01 (d, J = 4.8 Hz, 1H), 6.96 (d, J = 8.0 Hz, 1H), 6.90 (d, J = 8.4 Hz, 2H), 6.66-6.64 (dd, J₁ = 8.0 Hz & J₂ = 2.0 Hz, 1H), 6.56- 6.52 (m, 2H), 6.12 (s, 1H), 4.30 (s, 2H), 4.13 (t, J = 6.8 Hz, 2H), 4.07 (s, 2H), 3.10 (t, J = 6.8 Hz, 2H), 2.93 (s, 3H), 2.29 (s, 3H). m/z (M + H)⁺: 624.00 |
| 3) | 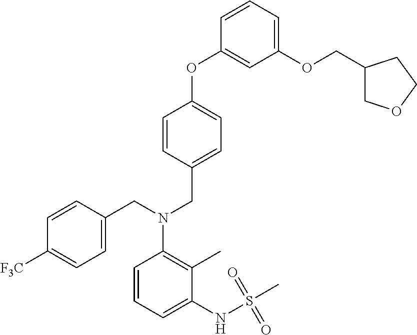 | N-(2-methyl-3-((4- (3-((tetrahydrofuran- 3- yl)methoxy)phenoxy) benzyl)(4- (trifluoromethyl) benzyl)amino)phenyl) methanesulfonamide | (CDCl₃, 400 MHz) δ 7.52 (d, J = 8.0 Hz, 2H), 7.32 (d, J = 8.0 Hz, 2H), 7.23-7.18 (m, 2H), 7.14-7.10 (m, 3H), 6.93-6.88 (m, 3H), 6.65- 6.62 (dd, J₁ = 2.0 Hz, J2 = 8.0 Hz, 1H), 6.57-6.53 (dd, J₁ = 2.0 Hz & J₂ = 8.0 Hz, 1H), 6.52 (t, J = 2.0 Hz, 1H), 6.19 (s, 1H), 4.11 (s, 2H), 4.01 (s, 2H), 3.90-3.85 (m, 4H), 3.83-3.75 (m, 1H), 3.70-3.66 (m, 1H), 2.96 (s, 3H), 2.73-2.68 (m, IH), 2.36 (s, 3H), 2.20-1.72 (m, IH), 1.75-1.66 (m, 1H). m/z (M + H)⁺: 641.10 |
| 4) | 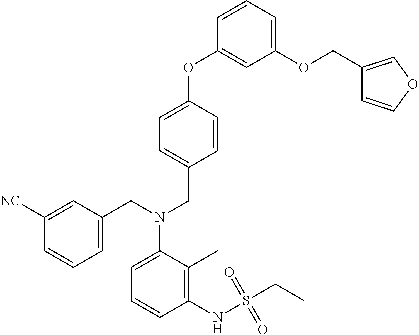 | N-(3-((3- cyanobenzyl)(4-(3- (furan-3- ylmethoxy)phenoxy) benzyl)amino)-2- methylphenyl)ethane sulfonamide | (CDCl₃, 400 MHz) δ 7.51-7.47 (m, 3H), 7.42-7.41 (m, 2H), 7.40 (d, J = 7.6 Hz, 1H), 7.22 (d, J = 6.8 Hz, 2H), 7.14-7.10 (m, 3H), 6.93 (d, J = 8.4 Hz, 2H), 6.86 (d, J = 7.6 Hz, 1H), 6.72-6.70 (m, 1H), 6.60-6.58 (m, 2H), 6.46 (s, 1H), 6.05 (s, 1H), 4.89 (s, 2H), 4.08 (s, 2H), 3.99 (s, 2H), 3.15-3.10 (q, J = 7.4 Hz, 2H), 2.35 (s, 3H), 1.36 (t, J = 7.4 Hz, 3H). m/z (M + H)⁺: 630.10 |
| 5) | 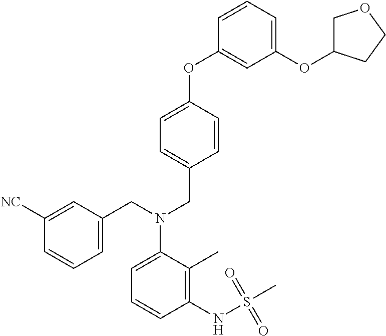 | N-(3-((3- cyanobenzyl)(4-(3- (tetrahydrofuran-3- yloxy)phenoxy) benzyl)amino)-2- methylphenyl) methanesulfonamide | (CDCl₃, 400 MHz) δ 7.52-7.49 (m, 2H), 7.43 (d, J = 8.4 Hz, 1H), 7.39- 7.37 (m, 1H), 7.23-7.20 (m, 2H), 7.14-7.13 (m, 3H), 6.93 (d, J = 6.8 Hz, 2H), 6.87 (d, J = 8.0 Hz, 1H), 6.61-6.57 (m, 2H), 6.48-6.47 (m, 1H), 6.15 (s, 1H), 4.87 (s, 1H), 4.09 (s, 2H), 4.00 (s, 2H), 3.98- 3.94 (m, 3H), 3.89-3.88 (m, 1H), 2.99 (s, 3H), 2.36 (s, 3H), 2.17- 2.14 (m, 2H). m/z (M + Na)⁺: 606.10 |
| 6) | 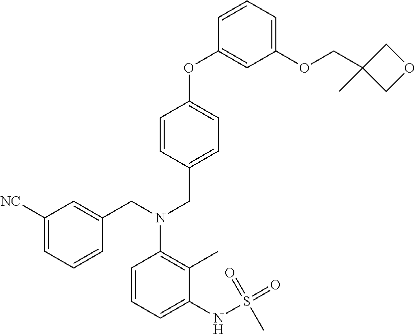 | N-(3-((3- cyanobenzyl)(4-(3- ((3-methyloxetan-3- yl)methoxy)phenoxy) benzyl)amino)-2- methylphenyl) methanesulfonamide | (CDCl₃, 400 MHz) δ 7.51 (d, J = 8.8 Hz, 2H), 7.43 (d, J = 7.6 Hz, 1H), 7.38-7.35 (m, 1H), 7.23-7.20 (m, 2H), 7.14-7.12 (m, 3H), 6.93 (d, J = 7.6 Hz, 2H), 6.87 (d, J = 7.6 Hz, 1H), 6.69-6.67 (dd, J₁ = 7.6 Hz & J₂ = 1.8 Hz, 1H), 6.60-6.57 (m, 2H), 6.21 (s, 1H), 4.60 (d, J = 6 Hz, 2H), 4.44 (d, J = 5.6 Hz, 2H), 4.11 (s, 2H), 4.00 (s, 2H), 3.98 (s, 2H), 2.99 (s, 3H), 2.35 (s, 3H), 1.46 (s, 3H). m/z (M + H)⁺: 597.93 |
| 7) | 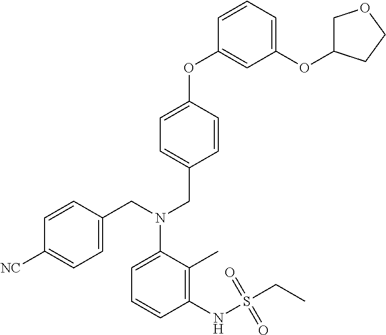 | N-(3-((4- cyanobenzyl)(4-(3- ((tetrahydrofuran-3- yl)oxy)phenoxy) benzyl)amino)-2- methylphenyl)ethane sulfonamide | (CDCl₃, 400 MHz) δ 7.55 (d, J = 8.0 Hz, 2H), 7.31 (d, J = 8.4 Hz, 2H), 7.23-7.19 (m, 2H), 7.14-7.07 (m, 3H), 6.93-6.91 (m, 2H), 6.83 (d, J = 8.0 Hz, 1H), 6.61-6.55 (m, 2H), 6.47 (t, J = 2.4 Hz, 1H), 6.13 (s, 1H), 4.88-4.86 (m, 1H), 4.11 (s, 2H), 3.99 (s, 2H), 3.97-3.93 (m, 3H), 3.91-3.87 (m, 1H), 3.12 (t, J = 7.4 Hz, 2H), 2.36 (s, 3H), 2.19- 2.12 (m, 2H), 1.36 (t, J = 7.4 Hz, 3H). m/z (M + H)⁺: 598.09 |
| 8) | 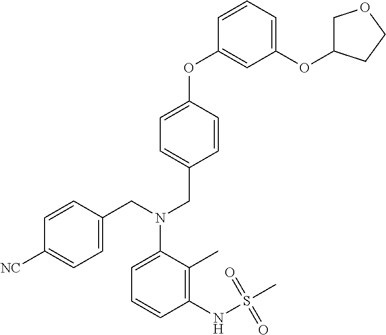 | N-(3-((4- cyanobenzyl)(4-(3- ((tetrahydrofuran-3- yl)oxy)phenoxy) benzyl)amino)-2- methylphenyl) methanesulfonamide | (CDCl₃, 400 MHz) δ 7.55 (d, J = 8.0 Hz, 2H), 7.32 (d, J = 8.0 Hz, 2H), 7.23-7.19 (m, 2H), 7.14-7.09 (m, 3H), 6.94-6.91 (m, 2H), 6.86 (d, J = 8.4 Hz, 1H), 6.60-6.55 (m, 2H), 6.47 (t, J = 2.4 Hz, 1H), 6.22 (s, 1H), 4.88-4.85 (m, 1H), 4.12 (s, 2H), 4.00 (s, 2H), 3.97-3.85 (m, 4H), 2.99 (s, 3H), 2.37 (s, 3H), 2.19-2.12 (m, 2H). m/z (M + H)⁺: 584.13 |
| 9) | 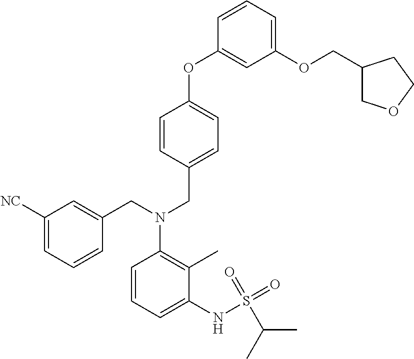 | N-(3-((3- cyanobenzyl)(4-(3- ((tetrahydrofuran-3- yl)methoxy)phenoxy) benzyl)amino)-2- methylphenyl) propane-2- sulfonamide | (CDCl₃, 400 MHz) δ 7.51-7.47 (m, 3H), 7.42-7.41 (m, 1H), 7.38-7.36 (m, 1H), 7.21 (t, J = 8.4 Hz, 1H), 7.14-7.06 (m, 3H), 6.92 (d, J = 8.4 Hz, 2H), 6.81 (d, J = 8.0 Hz, 1H), 6.65-6.63 (dd, J₁ = 8.0 Hz & J₂ = 2.0 Hz, 1H), 6.57-6.54 (m, 2H), 5.97 (s, 1H), 4.08 (s, 2H), 3.98 (s, 2H), 3.91-3.83 (m, 4H), 3.81-3.74 (m, 1H), 3.70-3.67 (m, 1H), 3.35- 3.32 (m, 1H), 2.74-2.69 (m, 1H), 2.35 (s, 3H), 2.11-2.07 (m, 1H), 1.73-1.69 (m, 1H), 1.38 (d, J = 6.8 Hz, 6H). m/z (M + Na)⁺: 648.00 |
| 10) | 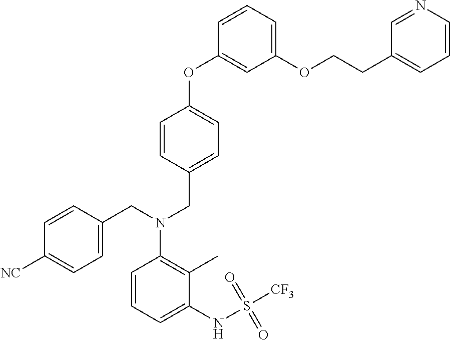 | N-(3-((4- cyanobenzyl)(4-(3- (2-(pyridin-3- yl)ethoxy)phenoxy) benzyl)amino)-2- methylphenyl)-1,1,1- trifluoro- methanesulfonamide | CDCl₃, 400 MHz δ 8.54 (d, 1H, J = 1.6 Hz), 8.48-8.50 (m, 1H), 7.60 (d, J = 8.0 Hz, 1H), 7.52 (d J = 8.4 Hz, 2H), 7.30 (d, J = 8.4 Hz, 2H), 7.23 (d, J = 2.8 Hz, 2H), 7.22 (s, 1H), 7.16-7.20 (m, 1H), 6.90 (d, J = 6.8 Hz, 2H), 6.87 (s, 1H), 6.14 (dd, J₁ = 2.0 & J₂ = 10.0 Hz, 1H), 6.55 (d, J = 8.4 Hz, 1H), 6.53-6.55 (m, 1H), 6.45 (d, J = 7.6 Hz, 1H), 6.41 (d, J = 8.0 Hz, 1H), 4.08-4.16 (m, 4H), 4.0 (s, 2H), 3.98 (s, 2H), 3.05 (t, J = 6.8 Hz, 2H), 2.24 (s, 3H). |
| 11) | 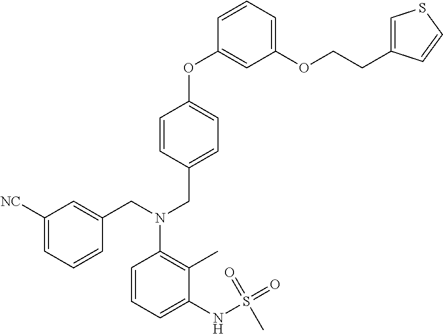 | N-(3-((3- cyanobenzyl)(4-(3- (2-(thiophen-3- yl)ethoxy)phenoxy) benzyl)amino)-2- methylphenyl) methanesulfonamide | (CDCl₃, 400 MHz) δ 7.52-7.49 (m, 2H), 7.42 (d, J = 7.6 Hz, 1H), 7.36 (t, J = 7.6 Hz, 1H), 7.27-7.26 (m, 1H), 7.23-7.19 (m, 2H), 7.13-7.10 (m, 3H), 7.07 (s, 1H), 7.01 (d, J = 4.8 Hz, 1H), 6.92 (d, J = 8.4 Hz, 2H), 6.85 (d, J = 8.0 Hz, 1H), 6.67- 6.65 (m, 1H), 6.57-6.54 (m, 2H), 6.15 (s, 1H), 4.15-4.08 (m, 2H), 4.05 (s, 2H), 3.99 (s, 2H), 3.10 (t, J = 6.8 Hz, 2H), 2.99 (s, 3H), 2.36 (s, 3H). m/z (M + Na)⁺: 646.80 |
| 12) | 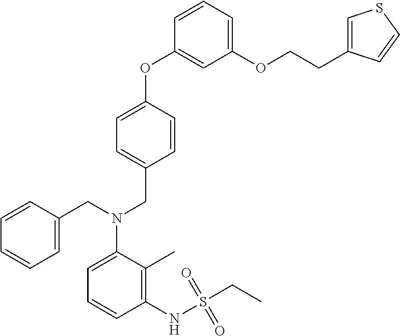 | N-(3-(benzyl(4-(3-(2- (thiophen-3- yl)ethoxy)phenoxy) benzyl)amino)-2- methylphenyl)ethane sulfonamide | (CDCl₃, 400 MHz) δ 7.23-7.18 (m, 6H), 7.17-7.10 (m, 4H), 7.08-6.99 (m, 3H), 6.91-6.85 (m, 3H), 6.66- 6.63 (m, 1H), 6.56-6.54 (m, 2H), 6.03 (s, 1H), 4.13 (t, J = 7.0 Hz, 2H), 4.04 (s, 2H), 4.00 (s, 2H), 3.13-3.05 (m, 4H), 2.34 (s, 3H), 1.34 (t, J = 7.2 Hz, 3H). m/z (M + Na)⁺: 634.50 |
| 13) | 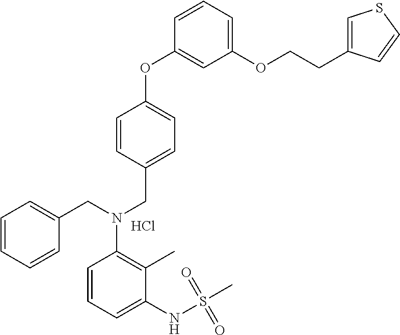 | N-(3-(benzyl(4-(3-(2- (thiophen-3- yl)ethoxy)phenoxy) benzyl)amino)-2- methylphenyl) methanesulfonamide hydrochloride | (DMSO-d₆, 400 MHz) δ 8.98 (s, 1H), 7.45-7.43 (m, 1H), 7.26-7.23 (m, 7H), 7.2-7.17 (m, 2H), 7.07- 7.05 (dd, J₁ = 4.8 Hz & J₂ = 1.2 Hz, 1H), 7.02-6.94 (m, 3H), 6.91- 6.89 (d, J = 8.4 Hz, 2H), 6.71-6.68 (dd, J₁ = 8.4 Hz & J₂ = 2.0 Hz, 1H), 6.51 (t, J = 2.4 Hz, 1H), 6.49- 6.42 (m, 1H), 4.13 (t, J = 6.6 Hz, 2H), 4.04 (s, 2H), 4.01 (s, 2H), 3.0 (t, J = 6.6 Hz, 2H), 2.89 (s, 3H), 2.38 (s, 3H). m/z (M + H)⁺: 598.90 |
| 14) | 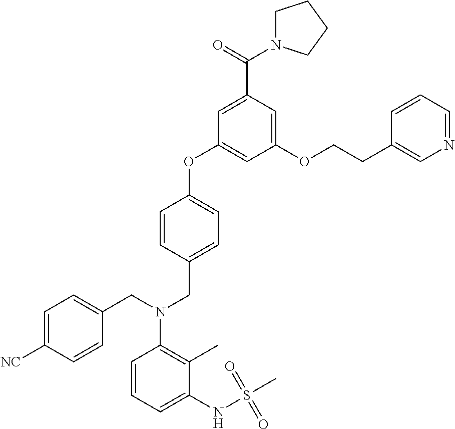 | N-(3-((4- cyanobenzyl)(4-(3- (2-(pyridin-3- yl)ethoxy)-5- (pyrrolidine-1- carbonyl)phenoxy) benzyl)amino)-2- methylphenyl) methanesulfonamide | (CDCl₃, 400 MHz) δ 8.52-8.49 (m, 2H), 7.59-7.56 (m, 3H), 7.37 (d, J = 8.4 Hz, 2H), 7.25-7.19 (m, 2H), 7.13-7.08 (m, 3H), 6.94 (d, J = 7.6 Hz, 1H), 6.89 (d, J = 8.8 Hz, 2H), 6.83 (s, 1H),6.73 (d, J = 1.2 Hz, 1H), 6.60 (t, J = 1.6 Hz, 1H), 6.50 (t, J = 2.2 Hz, 1H), 4.17 (t, J = 6.4 Hz, 2H), 4.13 (s, 2H), 3.99 (s, 2H), 3.59 (t, J = 6.8 Hz, 2H), 3.38 (t, J = 6.4 Hz, 2H), 3.06 (t, J = 6.4 Hz, 2H), 2.96 (s, 3H), 2.31 (s, 3H), 1.95-1.90 (m, 2H), 1.88-1.84 (m, 2H). m/z (M + H)⁺: 716.20 |
| 15) | 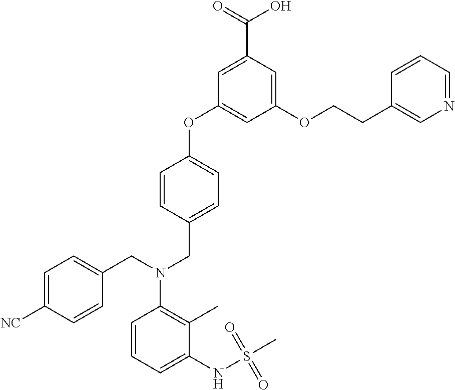 | 3-(4-(((4- cyanobenzyl)(2- methyl-3- (methylsulfonamido) phenyl)amino) methyl)phenoxy)- 5-(2-(pyridin-3- yl)ethoxy) benzoic acid | (CDCl₃, 400 MHz) δ 8.67 (s, 1H), 8.56 (d, J = 4.0 Hz, 1H), 7.71 (d, J = 7.6 Hz, 1H), 7.57 (d, J = 8.4 Hz, 2H), 7.39-7.33 (m, 4H), 7.21 (d, J = 8 Hz, 1H), 7.15 (d, J = 8.0 Hz, 1H), 7.12-7.08 (m, 3H), 6.97 (d, J = 8.0 Hz, 1H), 6.90-6.88 (m, 2H), 6.81 (bs, 1H), 6.72 (t, J = 2.2 Hz, 1H), 4.22 (t, J = 6.4 Hz, 2H), 4.14 (s, 2H), 3.99 (s, 2H), 3.12 (t, J = 6.4 Hz, 2H), 2.94 (s, 3H), 2.27 (s, 3H). m/z (M + H)⁺: 663.00 |
| 16) | 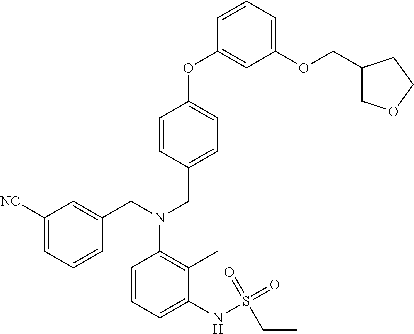 | N-(3-((3- cyanobenzyl)(4-(3- ((tetrahydrofuran-3- yl)methoxy)phenoxy) benzyl)amino)-2- methylphenyl)ethane sulfonamide | (CDCl₃, 400 MHz) δ 7.52-7.48 (m, 2H), 7.41-7.36 (m, 2H), 7.26-7.19 (m, 2H), 7.14-7.10 (m, 3H), 6.92 (d, J = 8.4 Hz, 2H), 6.83 (d, J = 8 Hz, 1H), 6.65-6.63 (dd, J₁ = 8.0 Hz & J₂ = 2.0 Hz, 1H), 6.57-6.53 (m, 2H), 6.13 (s, 1H), 4.08 (s, 2H), 3.99 (s, 2H), 3.87-3.83 (m, 4H), 3.78- 3.76 (m, 1H), 3.70-3.69 (m, 1H), 3.13 (q, J = 7.2 Hz, 2H), 2.71-2.66 (m, 1H), 2.36 (s, 3H), 2.10-2.08 (m, 1H), 1.73-1.69 (m, 1H), 1.36 (t, J = 7.6 Hz, 3H). m/z (M + Na)⁺: 634.00 |
| 17) | 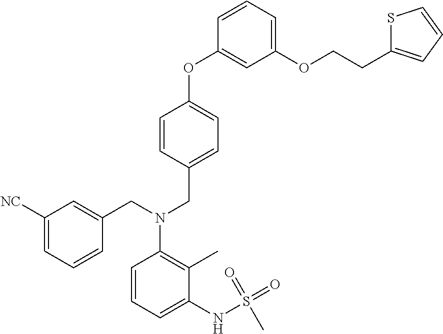 | N-(3-((3- cyanobenzyl)(4-(3- (2-(thiophen-2- yl)ethoxy)phenoxy) benzyl)amino)-2- methylphenyl) methanesulfonamide) | (CDCl₃, 400 MHz) δ 7.51-7.49 (m, 2H), 7.43-7.34 (m, 2H), 7.26-7.20 (m, 2H), 7.16-7.11 (m, 4H), 6.93- 6.84 (m, 5H), 6.67 (d, J = 7.6 Hz, 1H), 6.57 (d, J = 8.4 Hz, 2H), 6.14 (s, 1H), 4.15 (t, J = 6.2 Hz, 2H), 4.09 (s, 2H), 3.99 (s, 2H), 3.29 (t, J = 6.2 Hz, 2H), 2.99 (s, 3H), 2.36 (s, 3H). m/z (M + Na)⁺: 646.70 |
| 18) | 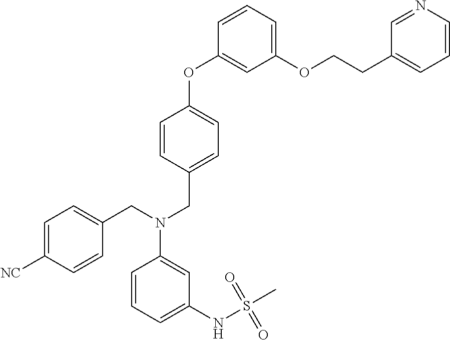 | N-(3-((4- cyanobenzyl)(4-(3- (2-(pyridin-3- yl)ethoxy)phenoxy) benzyl)amino)phenyl) methanesulfonamide | (CDCl₃, 400 MHz) δ 8.51-8.48 (m, 2H), 7.63-7.61 (m, 4H), 7.35 (d, J = 8.0 Hz, 2H), 6.63-6.58 (m, 3H), 6.51-6.49 (m, 2H), 6.45 (t, J = 2.2 Hz, 1H), 4.69 (s, 2H), 4.63 (s, 2H), 4.14 (t, J = 6.6 Hz, 2H), 3.06 (t, J = 6.6 Hz, 2H), 2.88 (s, 3H). m/z (M + H)⁺: 605.15 |
| 19) | 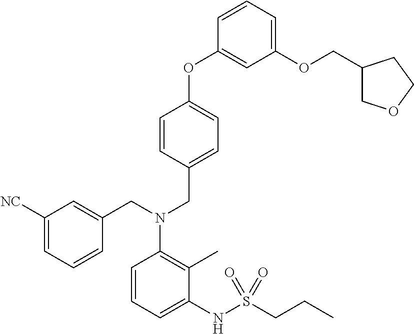 | N-(3-((3- cyanobenzyl)(4-(3- ((tetrahydrofuran-3- yl)methoxy)phenoxy) benzyl)amino)-2- methylphenyl) propane-1- sulfonamide | (CDCl₃, 400 MHz) δ 7.52-7.00 (m, 2H), 7.41-7.36 (m, 2H), 7.23-7.19 (m, 2H), 7.14-7.10 (m, 3H), 6.93 (d, J = 8.4 Hz, 2H), 6.83 (d, J = 8 Hz, 1H), 6.66-6.63 (dd, J₁ = 8.4 Hz & J₂ = 2.4 Hz, 1H), 6.58-6.53 (m, 2H), 6.1 (s, 1H), 4.08 (s, 2H), 3.99 (s, 2H), 3.91-3.83 (m, 4H), 3.70- 3.67 (m, 2H), 3.09-3.05 (m, 2H), 2.74-2.68 (m, 1H), 2.36 (s, 3H), 2.14-2.04 (m, 1H), 1.88-1.82 (m, 2H), 1.74-1.70 (m, 1H), 1.03 (t, J = 7.4 Hz, 3H). m/z (M + Na)⁺: 648.00 |
| 20) | 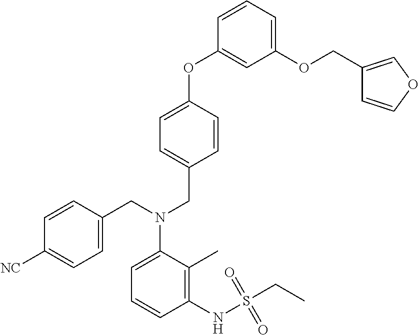 | N-(3-((4- cyanobenzyl)(4-(3- (furan-3- ylmethoxy)phenoxy) benzyl)amino)-2- methylphenyl)ethane sulfonamide | (CDCl₃, 400 MHz) δ 7.55 (d, J = 8.4 Hz, 2H), 7.47 (s, 1H), 7.42 (t, J = 1.6 Hz, IH), 7.31 (d, J = 8.4 Hz, 2H), 7.25-7.20 (m, 2H), 7.13-7.09 (m, 3H), 6.92 (d, J = 8.4 Hz, 2H), 6.81 (d, J = 7.6 Hz, 1H), 6.72-6.69 (m, 1H), 6.59-6.58 (m, 2H), 6.46 (s, 1H), 6.08 (s, 1H), 4.89 (s, 2H), 4.11 (s, 2H), 3.99 (s, 2H), 3.15- 3.10 (q, J = 7.4 Hz, 2H), 2.36 (s, 3H), 1.36 (t, J = 7.4 Hz, 3H). m/z (M + Na)⁺: 630.00 |
| 21) | 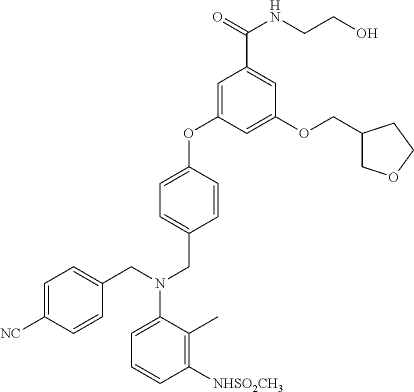 | 3-(4-(((4- cyanobenzyl)(2- methyl-3- (methylsulfonamido) phenyl)amino)methyl) phenoxy)-N-(2- hydroxyethyl)-5- ((tetrahydrofuran-3- yl)methoxy) benzamide | (CDCl₃, 400 MHz) δ 7.59 (d, J = 8.4 Hz, 2H), 7.44 (d, J = 8.0 Hz, 2H), 7.25-7.23 (m, 1H), 7.19 (t J = 8.0 Hz, 1H), 7.15 (s, 1H), 7.13- 7.11 (m, 1H), 7.05 (d, J = 8.4 Hz, 2H), 6.92 (t, J = 2.0 Hz, 1H), 6.87 (d, J = 8.4 Hz, 2H), 6.71 (t, J = 2.0 Hz, 1H), 6.69-6.68 (m, 1H), 6.52 (t, J = 5.2 Hz, 1H), 4.16 (s, 2H), 3.98 (s, 2H), 3.96-3.87 (m, 4H), 3.81-3.75 (m, 3H), 3.72-3.68 (dd, J₁ = 8.8 Hz & J₂ = 4.8 Hz, 1H), 3.62 (q, J = 5.2 Hz, 2H), 2.90 (s, 3H), 2.77-2.69 (m, 1H), 2.46-2.44 (m, 1H), 2.18 (s, 3H), 2.16-2.09 (m, 1H), 1.74-1.69 (m, 1H). m/z (M + H)⁺: 685.00 |
| 22) | 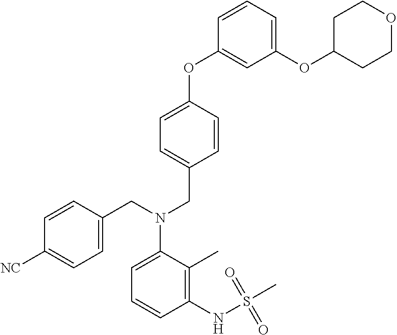 | N-(3-((4- cyanobenzyl)(4-(3- ((tetrahydro-2H- pyran-4- yl)oxy)phenoxy) benzyl)amino)-2- methylphenyl) methanesulfonamide | (CDCl₃, 400 MHz) δ 7.55 (d, J = 8.0 Hz, 2H), 7.31 (d, J = 8.0 Hz, 2H), 7.23-7.19 (m, 2H), 7.13-7.09 (m, 3H), 6.93-6.91 (m, 2H), 6.85 (d, J = 7.6 Hz, 1H), 6.67-6.65 (m, 1H), 6.56-6.53 (m, 2H), 6.18 (s, 1H), 4.46-4.42 (m, 1H), 4.12 (s, 2H), 3.98 (s, 2H), 3.97-3.93 (m, 2H), 3.58-3.52 (m, 2H), 3.00 (s, 3H), 2.37 (s, 3H), 2.04-1.97 (m, 2H), 1.81-1.72 (m, 2H). m/z (M + H)⁺: 598.27 |
| 23) | 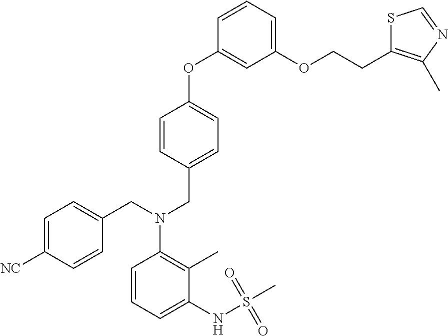 | N-(3-((4- cyanobenzyl)(4-(3- (2-(4-methythiazol- 5- yl)ethoxy)phenoxy) benzyl)amino)-2- methylphenyl) methanesulfonamide | (CDCl₃, 400 MHz) δ 8.59 (s, 1H), 7.56 d, J = 8.0 Hz, 2H), 7.32 (d, J = 8.0 Hz, 2H), 7.23-7.18 (m, 2H), 7.13-7.07 (m, 3H), 6.93 (d, J = 8.4 Hz, 2H), 6.86 (d, J = 8.0 Hz, 1H), 6.65-6.63 (dd, J₁ = 2.0 Hz & J₂ = 8.4 Hz, 1H), 6.58-6.55 (m, 1H), 6.52 (t, J = 2.4 Hz, 1H), 6.32 (s, 1H), 4.14-4.08 (m, 4H), 4.00 (s, 2H), 3.22 (t, J = 6.4 Hz, 2H), 2.99 (s, 3H), 2.41 (s, 3H), 2.37 (s, 3H). m/z (M + Na)⁺: 661.00 |
| 24) | 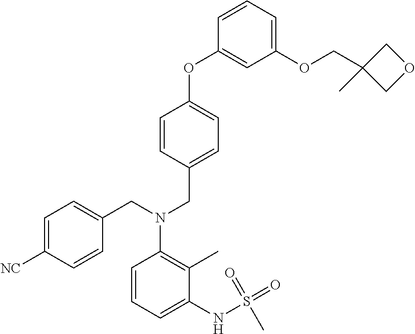 | N-(3-((4- cyanobenzyl)(4-(3- ((3-methyloxetan-3- yl)methoxy)phenoxy) benzyl)amino)-2- methylphenyl) methanesulfonamide | (CDCl₃, 400 MHz) δ 7.56 (d, J = 8.0 Hz, 2H), 7.32 (d, J = 8.4 Hz, 2H), 7.25-7.19 (m, 2H), 7.14-7.09 (m, 3H), 6.94-6.91 (m, 2H), 6.87 (d, J = 7.6 Hz, 1H), 6.69-6.67 (dd, J₁ = 1.6 Hz, J₂ = 8.4 Hz, 1H), 6.59- 6.56 (m, 2H), 6.24 (s, 1H), 4.60 (d, J = 6.0 Hz, 2H), 4.44 (d, J = 6.0 Hz, 2H), 4.12 (s, 2H), 4.00 (s, 2H), 3.97 (s, 2H), 2.99 (s, 3H), 2.36 (s, 3H), 1.41 (s, 3H). m/z (M + H)⁺: 598.21 |
| 25) | 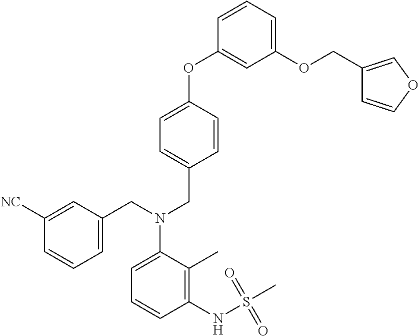 | N-(3-((3- cyanobenzyl)(4-(3- (furan-3- ylmethoxy)phenoxy) benzyl)amino)-2- methylphenyl) methanesulfonamide | (CDCl₃, 400 MHz) δ 7.52-7.47 (m, 3H), 7.43-7.42 (m, 2H), 7.38-7.34 (m, 1H), 7.23-7.20 (m, 2H), 7.14- 7.10 (m, 3H), 6.93 (d, J = 8.8 Hz, 2H), 6.86 (d, J = 8.0 Hz, 1H), 6.72- 6.69 (m, 1H), 6.60-6.58 (m, 2H), 6.46 (s, 1H), 6.14 (s, 1H), 4.89 (s, 2H), 4.09 (s, 2H), 3.99 (s, 2H), 2.99 (s, 3H), 2.36 (s, 3H). m/z (M + Na)⁺: 616.00 |
| 26) | 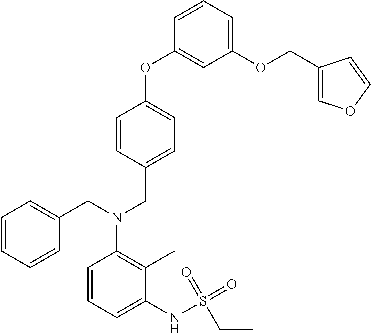 | N-(3-(benzyl(4-(3- (furan-3- ylmethoxy)phenoxy) benzyl)amino)-2- methylphenyl)ethane sulfonamide | (CDCl₃, 400 MHz) δ 7.46-7.41 (m, 2H), 7.28-7.25 (m, 1H), 7.23-7.19 (m, 6H), 7.14 (d, J = 8.8 Hz, 2H), 7.08 (t, J = 8.2 Hz, 1H), 6.90 (d, J = 8.4 Hz, 2H), 6.86 (d, J = 8.0 Hz, 1H), 6.70-6.68 (dd, J₁ = 8.0 Hz & J₂ = 2.0 Hz, 1H), 6.58-6.57 (m, 2H), 6.46 (s, 1H), 6.06 (s, 1H), 4.88 (s, 2H), 4.04 (s, 2H), 4.01 (s, 2H), 3.12-3.07 (m, 2H), 2.34 (s, 3H), 1.33 (t, J = 7.4 Hz, 3H). m/z (M + Na)⁺: 605.10 |
| 27) | 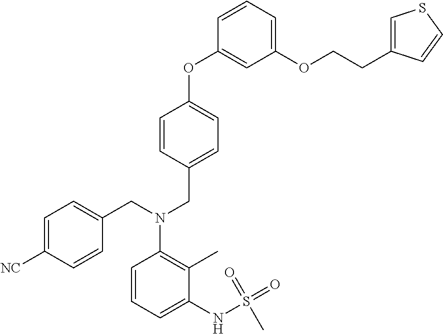 | N-(3-((4- cyanobenzyl)(4-(3- (2-(thiophen-3- yl)ethoxy)phenoxy) benzyl)amino)-2- methylphenyl) methanesulfonamide | (CDCl₃, 400 MHz) δ 7.55 (d, J = 8.4 Hz, 2H), 7.31 (d, J = 8.0 Hz, 2H), 7.27-7.19 (m, 3H), 7.12-7.06 (m, 4H), 7.02-7.01 (m, 1H), 6.91 (d, J = 8.4 Hz, 2H), 6.85 (d, J = 8.0 Hz, 1H), 6.67-6.65 (dd, J₁ = 2.4 Hz, J₂ = 8.8 Hz, 1H), 6.57-6.53 (m, 2H), 6.16 (s, 1H), 4.15-4.13 (m, 2H), 4.11 (s, 2H), 3.99 (s, 2H), 3.10 (t, J = 6.8 Hz, 2H), 2.99 (s, 3H), 2.36 (s, 3H). m/z (M + Na)⁺: 646.13 |
| 28) | 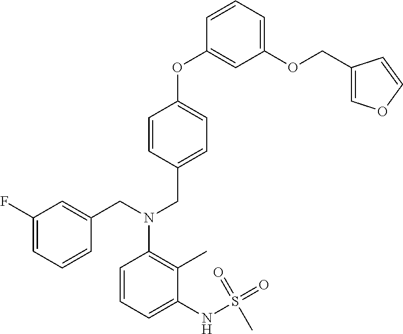 | N-(3-((3- fluorobenzyl)(4-(3- (furan-3- ylmethoxy)phenoxy) benzyl)amino)-2- methylphenyl) methanesulfonamide | CDCl₃, 400 MHz δ 7.47 (s, 1H), 7.41 (s, 1H), 7.19-7.22 (m, 2H), 7.09-7.14 (m, 3H), 6.97 (s, 1H), 6.87-6.94 (m, 4H), 6.68-6.71 (m, 1H), 6.57-6.59 (m, 2H), 6.46 (s, 1H), 6.16 (s, 1H), 4.89 (s, 2H), 4.05 (s, 2H), 4.01 (s, 2H), 2.97 (s, 3H), 2.04 (s, 3H). m/z (M + H)⁺: 587.10 |
| 29) | 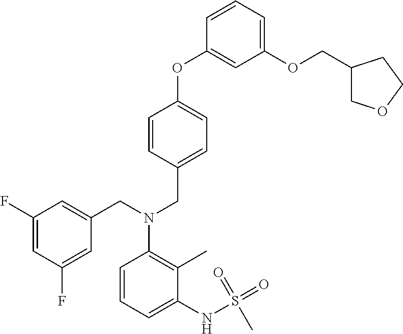 | N-(3-((3,5- difluorobenzyl)(4-(3- ((tetrahydrofuran-3- yl)methoxy)phenoxy) benzyl)amino)-2- methylphenyl) methanesulfonamide | (CDCl₃, 400 MHz) δ 7.23-7.19 (m, 2H), 7.14-7.10 (m, 3H), 6.91 (d, J = 8.4 Hz, 2H), 6.87 (d, J = 8.0 Hz, 1H), 6.75 (d, J = 6.0 Hz, 2H), 6.68- 6.62 (m, 2H), 6.57-6.52 (m, 2H), 6.16 (s, 1H), 4.04 (s, 2H), 4.00 (s, 2H), 3.91-3.83 (m, 3H), 3.81-3.73 (m, 2H), 3.70-3.66 (m, 1H), 2.98 (s, 3H), 2.73-2.69 (m, 1H), 2.37 (s, 3H), 2.13-1.05 (m, 1H), 1.75-1.67 (m, 1H). m/z (M + H)⁺: 609.00 |
| 30) | 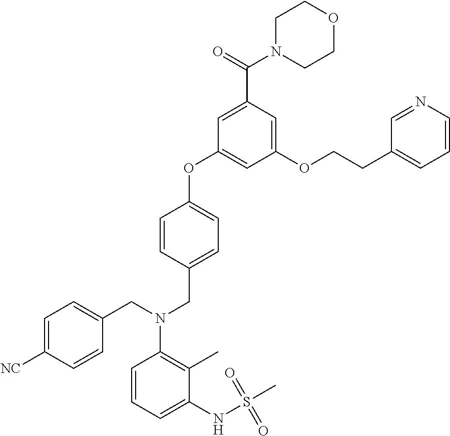 | N-(3-((4- cyanobenzyl)(4-(3- (morpholine-4- carbonyl)-5-(2- (pyridin-3- yl)ethoxy)phenoxy) benzyl)amino)-2- methylphenyl) methanesulfonamide | (DMSO-D₆, 400 MHz) δ 9.05 (s, 1H), 8.50 (d, J = 1.6 Hz, 1H), 8.42- 8.40 (dd, J₁ = 1.6 Hz & J₂ = 4.8 Hz, 1H), 7.73-7.70 (m, 3H), 7.45 (d, J = 8.4 Hz, 2H), 7.32-7.26 (m, 3H), 7.01-6.95 (m, 5H), 6.68-6.67 (m, 1H), 6.57 (t, J = 2.0 Hz, 1H), 6.45-6.44 (m, 1H), 4.20 (t, J = 6.4 Hz, 2H), 4.15 (s, 2H), 4.03 (s, 2H), 3.65-3.41 (m, 6H), 3.23-3.13 (m, 2H), 3.01 (t, J = 6.4 Hz, 2H), 2.90 (s, 3H), 2.37 (s, 3H). m/z (M + H)⁺: 732.40 |
| 31) | 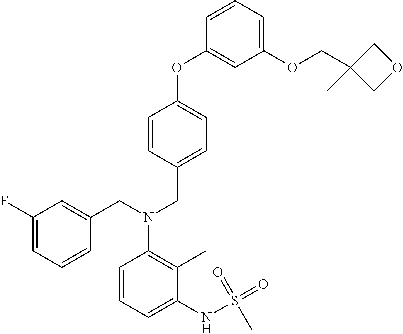 | N-(3-((3- fluorobenzyl)(4-(3- ((3-methyloxetan-3- yl)methoxy)phenoxy) benzyl)amino)-2- methylphenyl) methanesulfonamide | CDCl₃, 400 MHz δ 7.20-7.23 (m, 3H), 7.10-7.14 (m, 3H), 6.95-6.99 (m, 1H), 6.89-6.93 (m, 5H), 6.66 (dd, J₁ = 2.4 & J₂ = 8.0 Hz, 1H), 6.55-6.59 (m, 2H), 6.17 (s, 1H), 4.59 (d, J = 5.6 Hz, 2H), 4.43 (d, J = 6.0 Hz, 2H), 4.05 (s, 2H), 4.01 (s, 2H), 3.97 (s, 2H), 2.97 (s, 3H), 2.35 (s, 3H), 1.14 (s, 3H). m/z (M + H)⁺: 591.30 |
| 32) | 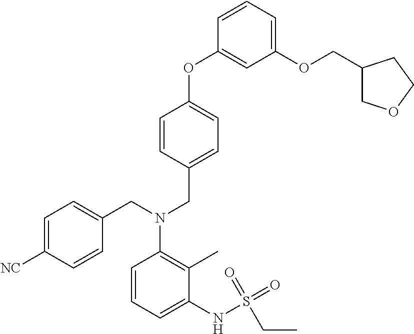 | N-(3-((4- cyanobenzyl)(4-(3- ((tetrahydrofuran-3- yl)methoxy)phenoxy) benzyl)amino)-2- methylphenyl)ethane sulfonamide | (CDCl₃, 400 MHz) δ 7.55 (d, J = 8.0 Hz, 2H), 7.31 (d, J = 8.0 Hz, 2H), 7.22-7.19 (m, 2H), 7.13-7.09 (m, 3H), 6.92 (d, J = 8.0 Hz, 2H), 6.83 (d, J = 7.8 Hz, 1H), 6.65-6.57 (dd, J₁ = 6.4 Hz & J₂ = 8.4 Hz, 1H), 6.57-6.52 (m, 2H), 6.08 (s, 1H), 4.11 (s, 2H), 3.99 (s, 2H), 3.90-3.83 (m, 5H), 3.70-3.67 (m, 1H), 3.15-3.10 (q, J = 7.2 Hz, 2H), 2.74-2.68 (m, 1H), 2.36 (s, 3H), 2.11-2.08 (m, 1H), 1.72-1.70 (m, 1H), 1.36 (t, J = 7.2 Hz, 3H). m/z (M + Na)⁺: 634.00 |
| 33) | 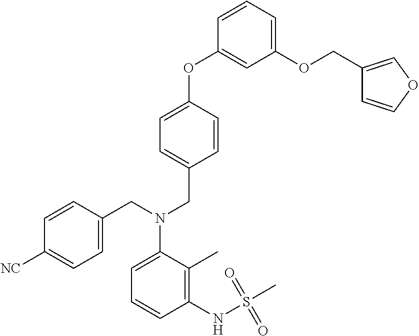 | N-(3-((4- cyanobenzyl)(4-(3- (furan-3- yl)methoxy)phenoxy) benzyl)amino)-2- methylphenyl) methanesulfonamide | (CDCl₃, 400 MHz) δ 7.55 (d, J = 8.0 Hz, 2H), 7.47-7.42 (m, 2H), 7.31 (d, J = 8.0 Hz, 2H), 7.21 (t, J = 8.0 Hz, 2H), 7.13-7.09 (m, 3H), 6.92 (d, J = 8.4 Hz, 2H), 6.85 (d, J = 7.6 Hz, 1H), 6.72-6.70 (m, 1H), 6.59-6.57 (m, 2H), 6.46 (s, 1H), 6.15 (s, 1H), 4.89 (s, 2H), 4.12 (s, 2H), 4.00 (s, 2H), 2.99 (s, 3H), 2.37 (s, 3H). m/z (M + Na)⁺: 616.10 |
| 34) | 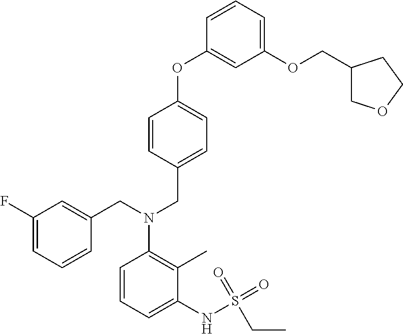 | N-(3-((3- fluorobenzyl)(4-(3- ((tetrahydrofuran-3- yl)methoxy)phenoxy) benzyl)amino)-2- methylphenyl)ethane sulfonamide | CDCl₃, 400 MHz δ 7.26 (m, 4H), 7.05 (m, 2H), 6.89 (m, 1H), 6.83 (m, 2H), 6.63 (d, J = 9.6 Hz, 1H), 6.5 (d, J = 7.6 Hz, 1H), 6.46 (s, 1H), 6.28 (s, 1H), 4.04 (s, 2H), 4.00 (s, 2H), 3.82 (m, 5H), 3.75 (m, 2H), 3.66 (m, 1H), 2.98 (s, 3H), 2.95 (s, 1H), 2.69 (m, 1H), 2.13 (s, 3H), 1.69 (m, 1H). m/z (M + H)⁺: 605.20 |
| 35) | 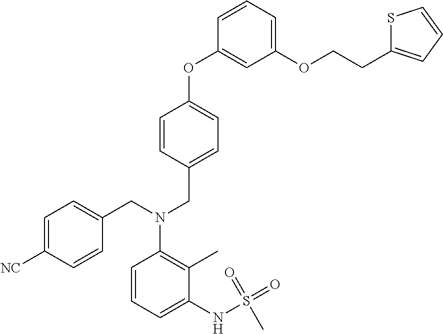 | N-(3-((4- cyanobenzyl)(4-(3- (2-(thiophen-2- yl)ethoxy)phenoxy) benzyl)amino)-2- methylphenyl) methanesulfonamide | (CDCl₃, 400 MHz) δ 7.55 (d, J = 8.0 Hz, 2H), 7.31 (d, J = 8.0 Hz, 2H), 7.23-7.19 (m, 2H), 7.16-7.09 (m, 4H), 6.95-6.89 (m, 4H), 6.85 (d, J = 8.0 Hz, 1H), 6.68-6.65 (m, 1H), 6.57-6.55 (m, 2H), 6.17 (s, 1H), 4.17-4.13 (m, 2H), 4.11 (s, 2H), 3.99 (s, 2H), 3.29 (t, J = 6.4 Hz, 2H), 2.99 (s, 3H), 2.37 (s, 3H) m/z (M + Na)⁺: 646.06 |
| 36) | 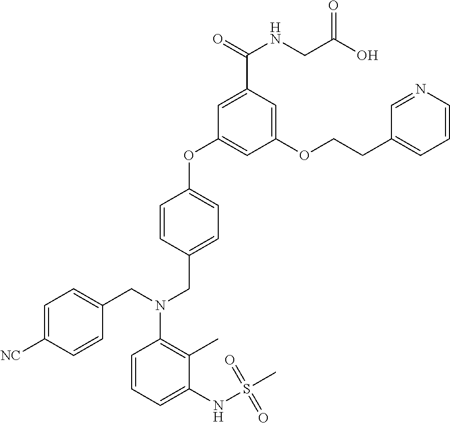 | 2-(3-(4-(((4- cyanobenzyl)(2- methyl-3- (methylsulfonamido) phenyl)amino)methyl) phenoxy)-5-(2- (pyridin-3- yl)ethoxy)benzamido) acetic acid | (DMSO-D₆, 400 MHz) δ 9.04 (s, 1H), 8.94 (t, J = 5.8 Hz, 1H), 8.90 (s, 1H), 8.76 (d, J = 5.2 Hz, 1H), 8.50 (d, J = 8.0 Hz, 1H), 8.14 (s, 1H), 7.96-7.93 (m, 1H), 7.72 (d, J = 8.4 Hz, 2H), 7.45 (d, J = 8.0 Hz, 2H), 7.28-7.25 (m, 3H), 7.04-7.00 (m, 2H), 6.97-6.93 (m, 3H), 6.73 (t, J = 2.2 Hz, 1H), 4.33 (t, J = 6.4 Hz, 2H), 4.15 (s, 2H), 4.03 (s, 2H), 3.84 (d, J = 5.6 Hz, 2H), 3.25 (t, J = 6.4 Hz, 2H), 3.14 (s, 3H), 2.91 (s, 3H). m/z (M + H)⁺: 720.10 |
| 37) | 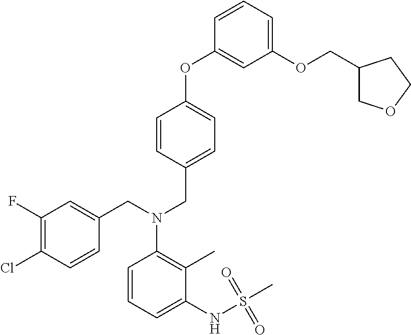 | N-(3-((4-chloro-3- fluorobenzyl)(4-(3- ((tetrahydrofuran-3- yl)methoxy)phenoxy) benzyl)amino)-2- methylphenyl) methanesulfonamide | (CDCl₃, 400 MHz) δ 7.29-7.27 (m, 1H), 7.23-7.19 (m, 1H), 7.14-7.10 (m, 3H), 7.02-6.99 (dd, J₁ = 1.6 Hz, J₂ = 10.0 Hz, 1H), 6.92 (d, J = 8.4 Hz, 3H), 6.86 (d, J = 8.0 Hz, 1H), 6.65-6.62 (dd, J₁ = 2.0 Hz & J₂ = 8-0 Hz, 1H), 6.57-6.55 (dd, J₁ = 2.0 Hz & J₂ = 8.4 Hz, 1H), 6.53 (t, J = 2.4 Hz, 1H), 6.18 (s, 1H), 4.02 (s, 2H), 3.99 (s, 3H), 3.91- 3.83 (m, 3H), 3.81-3.74 9 (m, 2H), 3.70-3.67 (m, 1H), 2.98 (s, 3H), 2.75-2.67 (m, IH), 2.36 (s, 3H), 2.11-2.05 (m, IH), 1.75-1.66 (m, 1H). m/z (M + H)⁺: 625.78 |
| 38) | 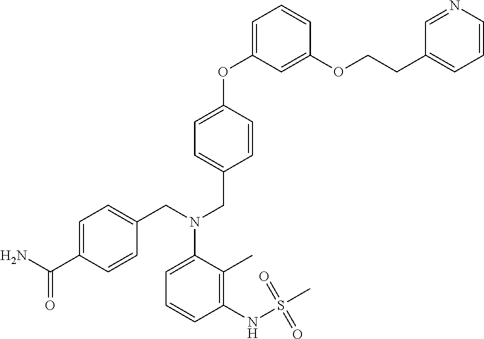 | 4-(((2-methyl-3- (methylsulfonamido) phenyl)(4-(3-(2- (pyridin-3- yl)ethoxy)phenoxy) benzyl)amino)methyl) benzamide | (DMSO-d₆, 400 MHz) δ 8.98 (s, 1H), 8.49 (d, J = 1.6 Hz, 1H), 8.41- 8.40 (m, 1H), 7.86 (s, 1H), 7.74 (d, J = 8.0 Hz, 2H), 7.74-7.72 (m, 1H), 7.31-7.20 (m, 7H), 7.015-6.99 (m, 1H), 6.96-6.90 (m, 4H), 6.68 (dd, J₁ = 8.4 Hz, J₂ = 2.0 Hz, 1H), 6.51 (t, J = 1.6 Hz, 1H), 6.47 (dd, J₁ = 8.0 Hz, J₂ = 2.0 Hz, 1H), 4.16 (t, J = 6.8 Hz, 2H), 4.10 (s, 2H), 4.02 (s, 2H), 3.01 (t, J = 6.4 Hz, 2H), 2.90 (s, 3H), 2.39 (s, 3H). m/z (M + H)⁺: 637.30 |
| 39) | 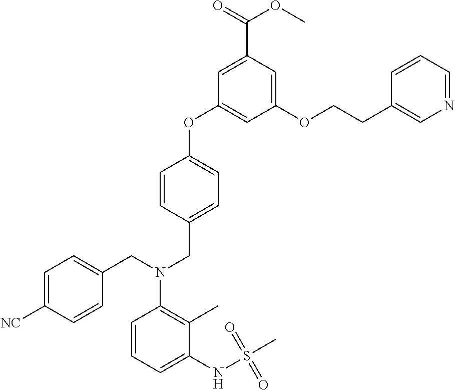 | methyl 3-(4-((4- cyanobenzyl)(2- methyl-3- (methylsulfonamido) phenyl)amino)methyl) phenoxy)-5-(2- (pyridin-3- yl)ethoxy)benzoate | (CDCl₃, 400 MHz) δ 8.53 (d, J = 1.6 Hz, 1H), 8.51-8.50 (dd, J₁ = 1.6 Hz, J₂ = 4.8 Hz, 1H), 7.62-7.57 (m, 3H), 7.38 (d, J = 8.4 Hz, 2H), 7.26- 7.21 (m, 3H), 7.16-7.09 (m, 4H), 6.96 (d, J = 8 Hz, 1H), 6.90-6.88 (m, 2H), 6.84 (s, 1H), 6.66 (t, J = 2Hz, 1H), 4.19 (t, J = 6.4 Hz, 2H), 4.13 (s, 2H), 4.00 (s, 2H), 3.88 (s, 3H), 3.08 (t, J = 6.4 Hz, 2H), 2.95 (s, 3H), 2.31 (s, 3H). m/z (M + H)⁺: 677.10 |
| 40) | 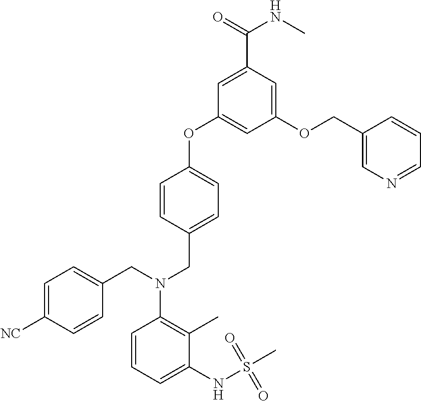 | 3-(4-(((4- cyanobenzyl)(2- methyl-3- (methylsulfonamido) phenyl)amino)methyl) phenoxy)-N-methyl- 5-(pyridin-3- ylmethoxy)benzamide | (CDCl₃, 400 MHz) δ 8.65 (s 1H), 8.60 (d, J = 4.8 Hz, 1H), 7.74 (d, J = 7.6 Hz, 1H), 7.58 (d, J = 8.4 Hz, 2H), 7.39 (d, J = 8.4 Hz, 2H), 7.35- 7.32 (m, 1H), 7.22 (d, J = 7.2 Hz, 1H), 7.15 (t, J = 8.0 Hz, 1H), 7.09 (d, J = 8.4 Hz, 2H), 7.04-7.00 (m, 2H), 6.89 (d, J = 8.4 Hz, 2H), 6.8l- 6.80 (m, 2H), 6.72 (t, J = 2.2 Hz, 1H), 6.08 (d, J = 4.4 Hz, 1H), 5.09 (s, 2H), 4.15 (s, 2H), 3.99 (s, 2H), 2.99 (d, J = 4.8 Hz, 3H), 2.95 (s, 3H), 2.27 (s, 3H). m/z (M + H)⁺: 661.90 |
| 41) | 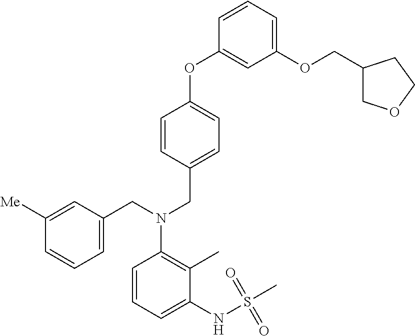 | N-(2-methyl-3-((3- methylbenzyl)(4-(3- ((tetrahydrofuran-3- yl)methoxy)phenoxy) benzyl)amino)phenyl) methanesulfonamide | (CDCl₃, 400 MHz) δ 7.22-7.18 (m, 2H), 7.15-7.08 (m, 4H), 7.04-7.00 (m, 3H), 6.90 (d, J = 8.4 Hz, 3H), 6.64-6.61 (dd, J₁ = 2.0 Hz, J₂ = 8.0 Hz, 1H), 6.56-6.54 (dd, J₁ = 1.6 Hz, J₂ = 8.0 Hz, 1H), 6.51 (t, J = 2.4 Hz, 1H), 6.15 (s, 1H), 4.01 (s, 4H), 3.90-3.84 (m, 3H), 3.82-3.73 (m, 2H), 3.69-3.66 (m, 1H), 2.95 (s, 3H), 2.66-2.74 (m, 1H), 2.35 (s, 3H), 2.30 (s, 3H), 2.11-2.04 (m, 1H), 1.75-1.66 (m, 1H). m/z (M + K)⁺: 624.88 |
| 42) | 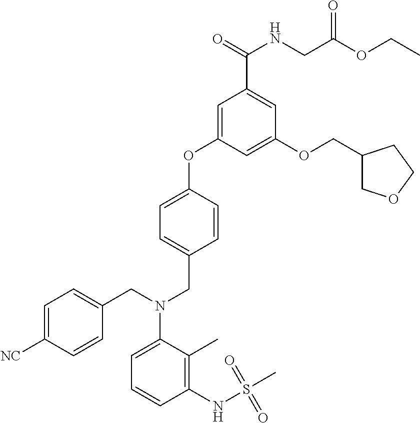 | ethyl 2-(3-(4-(((4- cyanobenzyl)(2- methyl-3- (methylsulfonamido) phenyl)amino)methyl) phenoxy)-5- ((tetrahydrofuran-3- yl)methoxy) benzamido)acetate | (CDCl₃, 400 MHz) δ 7.58 (d, J = 8.4 Hz, 2H), 7.40 (d, J = 8.4 Hz, 2H), 7.23 (d, J = 8.0 Hz, 1H), 7.16 (t, J = 8.0 Hz, 1H), 7.07 (d, J = 8.4 Hz, 2H), 7.03-7.00 (m, 2H), 6.89 (d, J = 8.4 Hz, 2H), 6.80 (s, 1H), 6.72-6.70 (m, 2H), 6.61-6.59 (m, 1H), 4.27-4.22 (m, 4H), 4.15 (s, 2H), 3.98 (s, 2H), 3.96-3.87 (m, 4H), 3.78 (q, J = 7.6 Hz, 1H), 3.71- 3.67 (dd, J₁ = 8.8 Hz, J₂ = 5.2 Hz, 1H), 2.92 (s, 3H), 2.75-2.70 (m, IH), 2.23 (s, 3H), 2.14-2.08 (m, 1H), 1.74-1.69 (m, 1H), 1.30 (t, J = 7.2 Hz, 3H). m/z (M + H)⁺: 728.03 |
| 43) | 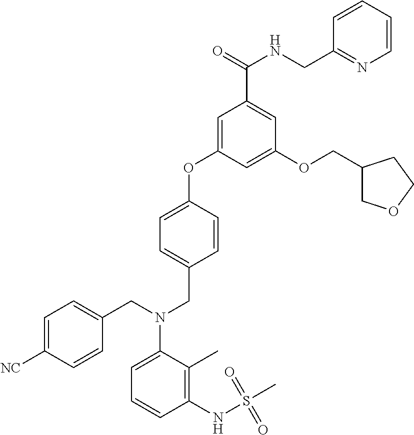 | 3-(4-(((4- cyanobenzyl)(2- methyl-3- (methylsulfonamido) phenyl)amino)methyl) phenoxy)-N- (pyridin-2-ylmethyl)- 5-((tetrahydrofuran- 3-yl)methoxy) benzamide | (CDCl₃, 400 MHz) δ 8.54 (d, J = 4.8 Hz, 1H), 7.68 (dt, J1 =7.6 Hz, J2 = 2.0 Hz, 1H), 7.58 (d, J = 8.4 Hz, 2H), 7.56-7.54 (m, 1H), 7.40 (d, J = 8.0 Hz, 2H), 7.35 (d, J = 8.0 Hz, 1H), 7.24-7.20 (m, 2H), 7.16 (t, J = 8.0 Hz, 1H), 7.08 (d, J = 8.4 Hz, 2H), 7.06-7.03 (m, 2H), 6.96 (s, 1H), 6.90 (d, J = 8.4 Hz, 2H), 6.84 (t, J = 2.0 Hz, 1H), 6.70 (t, J = 2.0 Hz, 1H), 4.75 (d, J = 4.8 Hz, 2H), 4.15 (s, 2H), 3.99 (s, 2H), 3.98-3.87 (m, 4H), 3.78 (q, J = 7.6 Hz, 1H), 3.72-3.68 (dd, J₁ = 8.8 Hz, J₂ = 5.2 Hz, 1H), 2.93 (s, 3H), 2.75-2.72 (m, 1H), 2.23 (s, 3H), 2.14-2.09 (m, 1H), 1.75-1.70 (m, 1H). m/z (M + H)⁺: 733.05 |
| 44) | 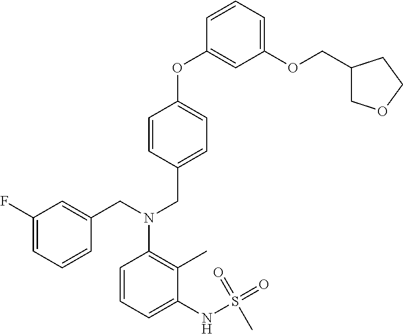 | N-(3-((3- fluorobenzyl)(4-(3- ((tetrahydrofuran-3- yl)methoxy)phenoxy) benzyl)amino)-2- methylphenyl) methanesulfonamide | CDCl₃, 400 MHz δ 7.18 (m, 5H), 6.97 (m, 2H), 6.92 (m, 2H), 6.87 (m, 3H), 6.63 (dd, J₁ = 1.6, J₂ = 8.8 Hz, 1H), 6.53 (dd, J₁ = 1.6 Hz, J₂ = 8.8 Hz, 1H), 6.16 (s, 1H), 4.04 (s, 2H), 4.00 (s, 2H), 3.85 (m, 4H), 3.76 (m, 1H), 3.66 (m, 1H), 2.98 (s, 3H), 2.90 (m, 1H), 2.28 (m, 1H), 2.04 (s, 3 H), 1.69 (m, 1H). m/z (M + H)⁺: 591.10 |
| 45) | 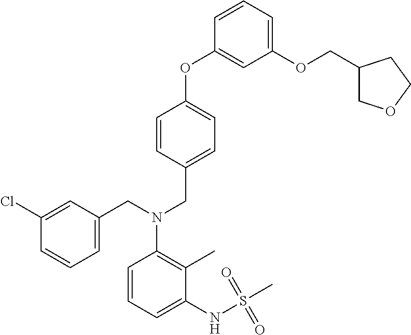 | N-(3-((3- chlorobenzyl)(4-(3- ((tetrahydrofuran-3- yl)methoxy)phenoxy) benzyl)amino)-2- methylphenyl) methanesulfonamide | (CDCl₃, 400 MHz) δ 7.23-7.18 (m, 5H), 7.17-7.12 (m, 3H), 7.10-7.06 (m, 1H), 6.92-6.88 (m, 3H), 6.64- 6.62 (m, 1H), 6.57-6.55 (m, 1H), 6.53 (t, J = 2.4 Hz, 1H), 6.17 (s, 1H), 4.03 (s, 2H), 4.00 (s, 2H), 3.91-3.83 (m, 3H), 3.81-3.73 (m, 2H), 3.70-3.66 (m, 1H), 2.96 (s, 3H), 2.75-2.69 (m, 1H), 2.35 (s, 3H), 2.10-2.07 (m, 1H), 1.73-1.68 (m, 1H). m/z (M + H)⁺: 607.19 |
| 46) | 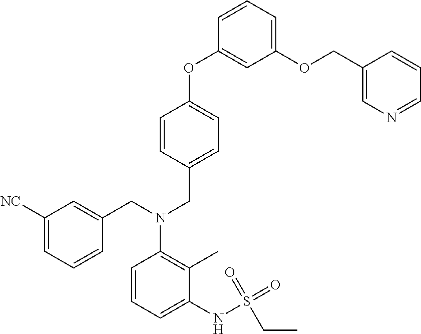 | N-(3-((3- cyanobenzyl)(4-(3- (pyridin-3- ylmethoxy)phenoxy) benzyl)amino)-2- methylphenyl)ethane sulfonamide | (CDCl₃, 400 MHz) δ 8.64 (d, J = 2.0 Hz, 1H), 8.59-8.57 (dd, J₁ = 4.4 Hz, J₂ = 1.2 Hz, 1H), 7.75 (d, J = 8.0 Hz, 1H), 7.52-7.49 (m, 2H), 7.41-7.38 (m, 1H), 7.35 (d, J = 7.8 Hz, 1H), 7.33-7.30 (m, 1H), 7.24- 7.21 (m, 2H), 7.14-7.10 (m, 3H), 6.92 (d, J = 8.4 Hz, 2H), 6.84 (d, J = 7.6 Hz, 1H), 6.73-6.70 (m, 1H), 6.62-6.59 (m, 2H), 6.17 (s, 1H), 5.04 (s, 2H), 4.08 (s, 2H), 3.99 (s, 2H), 3.15-3.10 (q, J = 7.6 Hz, 2H), 2.35 (s, 3H), 1.36 (t, J = 7.6 Hz, 3H). m/z (M + Na)⁺: 641.10 |
| 47) | 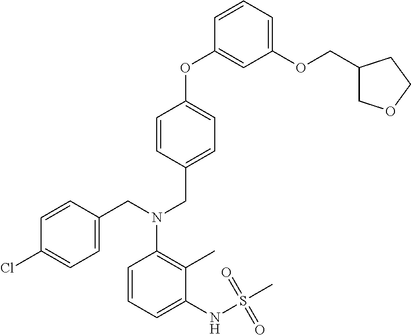 | N-(3-((4- chlorobenzyl)(4-(3- ((tetrahydrofuran-3- yl)methoxy)phenoxy) benzyl)amino)-2- methylphenyl) methanesulfonamide | (CDCl₃, 400 MHz) δ 7.23-7.18 (m, 4H), 7.15-7.09 (m, 5H), 6.92-6.86 (m, 3H), 6.64-6.62 (dd, J₁ = 2.0 Hz, J₂ = 8.0 Hz, 1H), 6.57-6.54 (dd, J₁ = 1.6 Hz, J₂ = 8.4 Hz, 1H), 6.52 (t, J = 2.0 Hz, 1H), 6.17 (s, 1H), 4.01 (s, 2H), 3.99 (s, 2H), 3.91-3.83 (m, 3H), 3.81-3.73 (m, 2H), 3.70-3.66 (m, 1H), 2.96 (s, 3H), 2.73-2.68 (m, 1H), 2.34 (s, 3H), 2.13-2.06 (m, 1H), 1.73-1.68 (m, 1H). m/z (M + H)⁺: 608.15 |
| 48) | 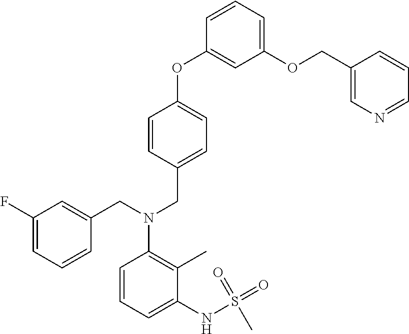 | N-(3-((3- fluorobenzyl)(4-(3- (pyridin-3- ylmethoxy)phenoxy) benzyl)amino)-2- methylphenyl) methanesulfonamide | CDCl₃, 400 MHz δ 8.64 (s, 1H), 8.57 (d, J = 4.4 Hz, 1H), 7.74 (d, J = 8.0 Hz, 1H), 7.30 (q, J = 7.6 Hz, 1H), 7.26 (s, 1H), 7.19 (t, J = 3.6 Hz, 1H), 7.09-7.15 (m, 3H), 6.95 (t, J = 8.4 Hz, 1H), 6.88-6.92 (m, 5H), 6.71 (dd, J₁ = 0.8, J₂ = 9.6 Hz, 1H), 6.58 (d, J = 2.4 Hz, 2H), 6.29 (s, 1H), 5.04 (s, 2H), 4.05 (s, 2H), 3.99 (s, 2H), 2.94 (s, 3H), 2.25 (s, 3H). m/z (M + H)⁺: 598.24 |
| 49) | 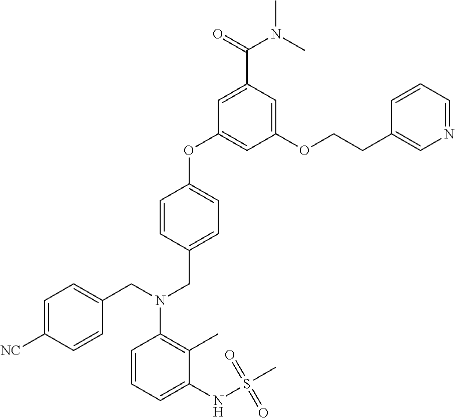 | 3-(4-(((4- cyanobenzyl)(2- methyl-3- (methylsulfonamido) phenyl)amino)methyl) phenoxy)-N,N- dimethyl-5-(2- (pyridin-3- yl)ethoxy)benzamide | (CDCl₃, 400 MHz) δ 8.50 (d, J = 6.0 Hz, 2H), 7.60-7.56 (m, 3H), 7.36 (d, J = 8.4 Hz, 2H), 7.25-7.19 (m, 2H), 7.14-7.09 (m, 3H), 6.94- 6.88 (m, 3H), 6.76 (s, 1H), 6.63- 6.62 (m, 1H), 6.52-6.51 (m, 1H), 6.49 (t, J = 2.2 Hz, 1H), 4.15 (t, J = 6.6 Hz, 2H), 4.11 (s, 2H), 4.00 (s, 2H), 3.08-3.05 (m, 5H), 2.99 (s, 3H), 2.95 (s, 3H), 2.32 (s, 3H). m/z (M + H)⁺: 689.10 |
| 50) | 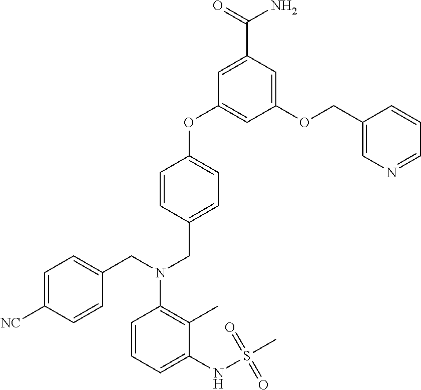 | 3-(4-(((4- cyanobenzyl)(2- methyl-3- (methylsulfonamido) phenyl)amino)methyl) phenoxy)-5- (pyridin-3- ylmethoxy)benzamide | (CDCl₃, 400 MHz) δ 8.66 (d, J = 2.0 Hz, 1H), 8.61-8.59 (dd, J₁ = 1.6 Hz, J₂ = 4.8 Hz, 1H), 7.76-7.74 (td, J₁ = 2.0 Hz, J₂ = 7.6 Hz, 1H), 7.59 (d, J = 8.4 Hz, 2H), 7.42 (d, J = 8.4 Hz, 2H), 7.35-7.32 (m, 1H), 7.24- 7.22 (m, 1H), 7.15 (t, J = 8 Hz, 1H), 7.09-7.07 (m, 3H), 7.04-7.02 (m, 1H), 6.90-6.86 (m, 2H), 6.79-6.78 (m, 1H), 6.77 (t, J = 2.4 Hz, 1H), 6.02 (bs, 1H), 5.74 (bs, 1H), 5.09 (s, 2H), 4.15 (s, 2H), 3.98 (s, 2H), 2.95 (s, 3H), 2.57 (s, 3H). m/z (M + H)⁺: 648.00 |
| 51) | 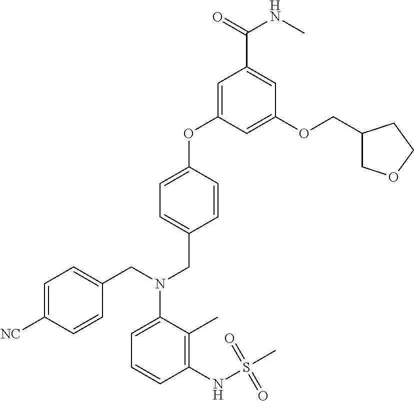 | 3-(4-(((4- cyanobenzyl)(2- methyl-3- (methylsulfonamido) phenyl)amino)methyl) phenoxy)-N-methyl- 5-((tetrahydrofuran- 3-yl)methoxy) benzamide | (CDCl₃, 400 MHz) δ 7.58 (d, J = 8.0 Hz, 2H), 7.39 (d, J = 8.0 Hz, 2H), 7.22 (d, J = 8.0 Hz, 1H), 7.15 (t, J = 8.0 Hz, 1H), 7.08 (d, J = 8.4 Hz, 2H), 7.01 (d, J = 8.0 Hz, 1H), 6.94 (s, 1H), 6.89 (d, J = 8.4 Hz, 2H), 6.76 (s, 2H), 6.68 (t, J = 2.0 Hz, 1H), 6.08 (brd, J = 4.8 Hz, 1H), 4.15 (s, 2H), 3.98 (s, 2H), 3.96-3.86 (m, 4H), 3.80-3.74 (m, 1H), 3.71-3.67 (m, 1H), 2.99 (d, J = 4.8 Hz, 3H), 2.95 (s, 3H), 2.75- 2.69 (m, 1H), 2.26 (s, 3H), 2.15- 2.04 (m, 1H), 1.75-1.67 (m, 1H). m/z (M + H)⁺: 655.24 |
| 52) | 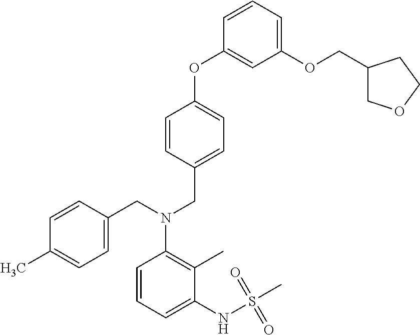 | N-(2-methyl-3-((4- methylbenzyl)(4-(3- ((tetrahydrofuran-3- yl)methoxy)phenoxy) benzyl)amino)phenyl) methanesulfonamide | (CDCl₃, 400 MHz) δ 7.22-7.18 (m, 2H), 7.15-7.10 (m, 3H), 7.09-7.05 (m, 4H), 6.91-6.88 (m, 3H), 6.64- 6.61 (dd, J₁ = 8.0 Hz, J₂ = 2.0 Hz, 1H), 6.56-6.54 (dd, J₁ = 8.0 Hz, J₂ = 1.6 Hz, 1H), 6.51 (t, J = 2.4 Hz, 1H), 6.18 (s, 1H),4.00(s, 4H), 3.91-3.82 (m, 4H), 3.80-3.73 (m, 1H), 3.70-3.66 (m, 1H), 2.95 (s, 3H), 2.72-2.69 (m, 1H), 2.34 (s, 3H), 2.31 (s, 3H), 2.13-2.06 (m, 1H), 2.04-1.66 (m, 1H). m/z (M + H)⁺: 587.24 |
| 53) | 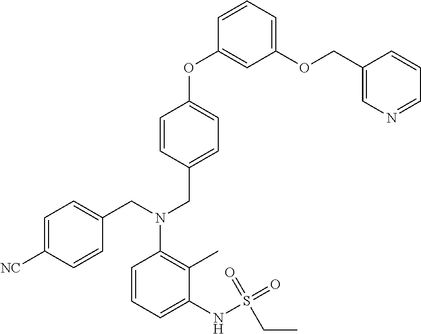 | N-(3-((4- cyanobenzyl)(4-(3- (pyridin-3- ylmethoxy)phenoxy) benzyl)amino)-2- methylphenyl)ethane sulfonamide | (CDCl₃, 400 MHz) δ 8.64 (d, J = 1.6 Hz, 1H), 8.59-8.57 (m, 1H), 7.74-7.75 (m, 1H), 7.55 (d, J = 8.4 Hz, 2H), 7.33-7.30 (m, 3H), 7.24-7.19 (m, 2H), 7.14-7.07 (m, 3H), 6.92 (d, J = 8.4 Hz, 2H), 6.84 (d, J = 8 Hz, 1H), 6.73-6.70 (m, 1H), 6.61-6.59 (m, 2H), 6.17 (s, 1H), 5.04 (s, 2H), 4.11 (s, 2H), 3.99 (s, 2H), 3.15-3.09 (q, J = 7.4 Hz, 2H), 2.36 (s, 3H), 1.36 (t, J = 7.4 Hz, 3H). m/z (M + H)⁺: 619.10 |
| 54) | 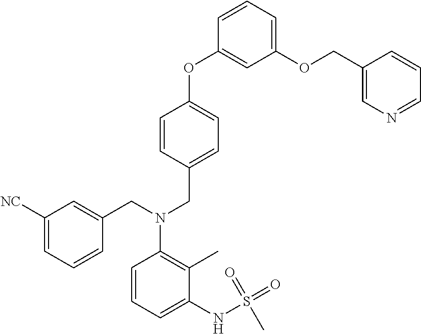 | N-(3-((3- cyanobenzyl)(4-(3- (pyridin-3- ylmethoxy)phenoxy) benzyl)amino)-2- methylphenyl) methanesulfonamide | (CDCl₃, 400 MHz) δ 8.64-8.59 (m, 1H), 8.59-8.57 (m, 1H), 7.75 (d, J = 8 Hz, 1H), 7.52-7.50 (m, 2H), 7.43 (d, J = 8 Hz, 1H), 7.37 (d, J = 7.2 Hz, 1H), 7.34-7.31 (m, 1H), 7.24-7.20 (m, 2H), 7.14-7.10 (m, 3H), 6.92 (d, J = 8.4 Hz, 2H), 6.87 (d, J = 8.6 Hz, 1H), 6.73-6.70 (dd, J₁ = 8.4 Hz, J₂ = 2.2 Hz, 1H), 6.62 (d, J = 1.6 Hz, 1H), 6.60-6.58 (m, 1H), 6.27 (s, 1H), 5.04 (s, 2H), 4.09 (s, 2H), 4.00 (s, 2H), 2.99 (s, 3H), 2.36 (s, 3H). m/z (M + Na)⁺: 606.10 |
| 55) | 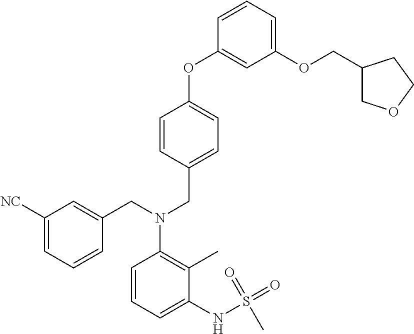 | N-(3-((3- cyanobenzyl)(4-(3- ((tetrahydrofuran-3- yl)methoxy)phenoxy) benzyl)amino)-2- methylphenyl) methanesulfonamide | (CDCl₃, 400 MHz) δ 7.52-7.49 (m, 2H), 7.42-7.36 (m, 2H), 7.26-7.19 (m, 2H), 7.14-7.12 (m, 3H), 6.92 (d, J = 8.4 Hz, 2H), 6.86 (d, J = 8 Hz, 1H), 6.65-6.63 (m, 1H), 6.57- 6.53 (m, 2H), 6.37 (s, 1H), 4.09 (s, 2H), 3.99 (s, 2H), 3.91-3.83 (m, 4H), 3.77-3.69 (m, 2H), 2.99 (s, 3H), 2.76-2.69 (m, 1H), 2.37 (s, 3H), 2.13-2.05 (m, 1H), 1.75-1.64 (m, 1H). m/z (M + Na)⁺: 619.50 |
| 56) | 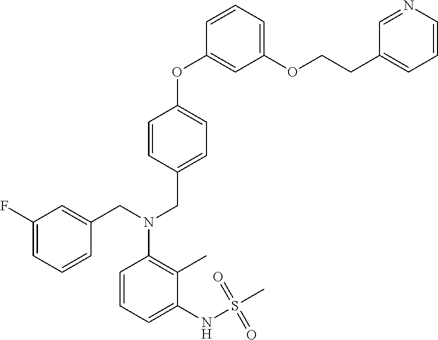 | N-(3-((3- fluorobenzyl)(4-(3- (2-(pyridin-3- yl)ethoxy)phenoxy) benzyl)amino)-2- methylphenyl) methanesulfonamide | CD₃OD, 400 MHz δ 9.88 (s, 1H), 8.39 (d, J = 8.0 Hz, 1H), 7.79-7.81 (m, 1H), 7.26-7.28 (m, 1H), 7.23- 7.26 (m, 1H), 7.17-7.21 (m, 3H), 7.04-7.09 (m, 3H), 6.96-6.99 (m, 2H), 6.91-6.94 (m, 1H), 6.85-6.88 (m, 2H), 6.63 (dd, J₁ = 1.6, J₂ = 8.4 Hz, 1H), 6.48-6.50 (m, 1H), 6.462- 6.467 (m, 1H), 4.21-4.24 (m, 2H), 4.11 (m, 2H), 4.07 (s, 2H), 3.07- 3.13 (m, 2H), 2.90 (s, 3H), 2.43 (s, 3H). m/z (M + H)⁺: 612.20 |
| 57) | 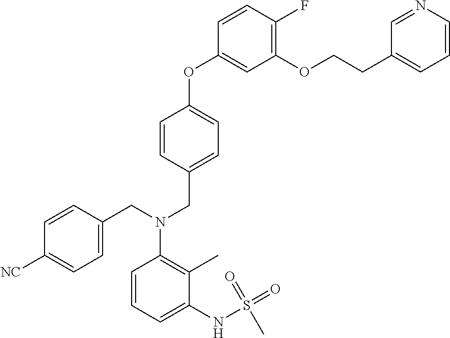 | N-(3-((4- cyanobenzyl)(4-(4- fluoro-3-(2-(pyridin- 3- yl)ethoxy)phenoxy) benzyl)amino)-2- methylphenyl) methanesulfonamide | (CDCl₃, 400 MHz,) δ 8.53-8.50 (m, 2H), 7.66 (d, J = 8.0 Hz, 1H), 7.56 (d, J = 8 Hz, 2H), 7.33 (d, J = 8.0 Hz, 2H), 7.25 (s, 1H), 7.20 (d, J = 8.0 Hz, 1H), 7.13-7.11 (m, 3H), 7.05-7.00 (m, 1H), 6.87-6.85 (m, 3H), 6.62-6.59 (dd, J₁ = 8.8 Hz, J₂ = 2.6 Hz, 1H), 6.52-6.49 (m, 1H), 6.40 (s, 1H), 4.16 (t, J = 6.8 Hz, 2H), 4.12 (s, 2H), 4.00 (s, 2H), 3.12 (t, J = 6.8 Hz, 2H), 3.01 (s, 3H), 2.38 (s, 3H). m/z (M + H)⁺: 637.32 |
| 58) | 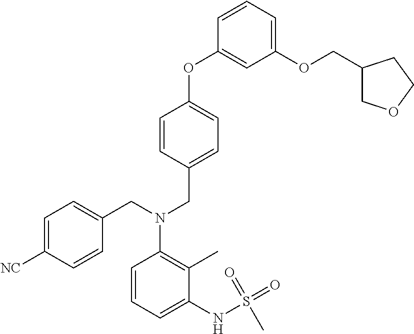 | N-(3-((4- cyanobenzyl)(4-(3- ((tetrahydrofuran-3- yl)methoxy)phenoxy) benzyl)amino)-2- methylphenyl) methanesulfonamide | (CDCl₃, 400 MHz) δ 7.55 (d, J = 8.4 Hz, 2H), 7.32 (d, J = 8.0 Hz, 2H), 7.23-7.19 (m, 2H), 7.17-7.09 (m, 3H), 6.92 (d, J = 8.8 Hz, 2H), 6.86 (d, J = 8.0 Hz, 1H), 6.65-6.63 (dd, J₁ = 2.0 Hz, J₂ = 8.4 Hz, 1H), 6.57-6.55 (dd, J₁ = 1.6 Hz, J₂ = 8.0 Hz, 1H), 6.52 (t, J = 2.4 Hz, 1H), 6.20 (s, 1H), 4.12 (s, 2H), 3.99 (s, 2H), 3.91-3.73 (m, 5H), 3.70-3.66 (m, 1H), 2.99 (s, 3H), 2.74-2.68 (m, 1H), 2.37 (s, 3H), 2.13-2.04 (m, 1H), 1.75-1.72 (m, 1H). m/z (M + Na)⁺: 620.00 |
| 59) | 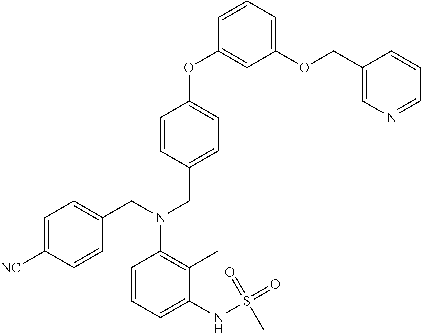 | N-(3-((4- cyanobenzyl)(4-(3- (pyridin-3- ylmethoxy)phenoxy) benzyl)amino)-2- methylphenyl) methanesulfonamide | (CDCl₃, 400 MHz) δ 8.65 (d, J = 1.6 Hz, 1H), 8.59-8.57 (m, 1H), 7.73-7.75 (m, 1H), 7.56 (d, J = 8.4 Hz, 2H), 7.33-7.30 (m, 3H), 7.26- 7.18 (m, 2H), 7.14-7.11 (m, 3H), 6.93-6.90 (m, 2H), 6.86 (d, J = 8.0 Hz, 1H), 6.73-6.70 (dd, J₁ = 2.4 Hz, J₂ = 8.4 Hz, 1H), 6.16.61-6.58 (m, 2H), 6.29 (s, 1H), 5.04 (s, 2H), 4.12 (s, 2H), 4.00 (s, 2H), 2.99 (s, 3H), 2.37 (s, 3H). m/z (M + H)⁺: 605.00 |
| 60) | 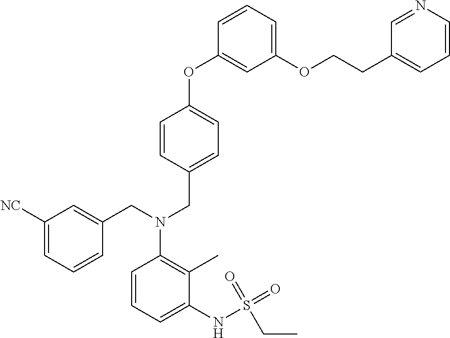 | N-(3-((3- cyanobenzyl)(4-(3- (2-(pyridin-3- yl)ethoxy)phenoxy) benzyl)amino)-2- methylphenyl)ethane sulfonamide | (DMSO-d₆, 400 MHz) δ 8.98 (s, 1H), 8.53-8.31 (m, 2H), 7.71 (d, J = 8.0 Hz, 1H), 7.65-7.53 (m, 5H), 7.46 (t, J = 8 Hz, 1H), 7.32-7.20 (m, 4H), 7.01-6.89 (m, 4H), 7.70- 7.67 (dd, J₁ = 8.0 Hz, J₂ = 2.0 Hz, 1H), 6.51-6.46 (m, 2H), 4.16 (t, J = 6.7 Hz, 2H), 4.11 (s, 2H), 4.02 (s, 2H), 3.02-2.97 (m, 4H), 2.37 (s, 3H), 1.19 (t, J = 7.4 Hz, 3H). m/z (M + H)⁺: 633.10 |
| 61) | 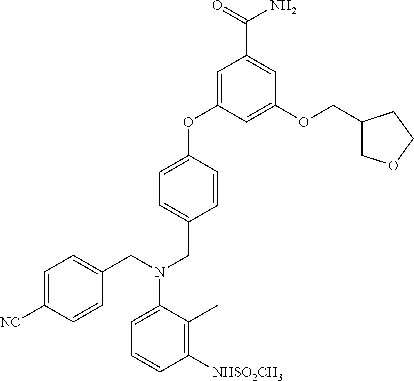 | 3-(4-(((4- cyanobenzyl)(2- methyl-3- (methylsulfonamido) phenyl)amino)methyl) phenoxy)-5- ((tetrahydrofuran-3- yl)methoxy) benzamide | (CDCl₃, 400 MHz) δ 7.59 (d, J = 8.4 Hz, 2H), 7.41 (d, J = 8.4.0 Hz, 2H), 7.24 (d, J = 8.0 Hz, 1H), 7.16 (t, J = 8.0 Hz, 1H), 7.07 (d, J = 8.8 Hz, 2H), 7.03 (d, J = 8.0 Hz, 1H), 6.98-6.97 (m, 1H), 6.89 (d, J = 8.4 Hz, 2H), 6.80 (s, 1H), 6.75-6.74 (m, 1H), 6.72-6.71 (m, 1H), 6.0 (br s, 1H), 5.7 (br s, 1H), 4.15 (s, 2H), 3.98 (s, 2H), 3.98-3.87 (m, 4H), 3.78 (q, J = 7.6 Hz, 1H), 3.71-3.67 (dd, J₁ = 8.8 Hz, J₂ = 5.2 Hz, 1H), 2.95 (s, 3H), 2.75-2.70 (m, 1H), 2.24 (s, 3H), 2.13-2.08 (m, 1H), 1.74-1.69 (m, 1H). m/z (M + H)⁺: 641.87 |
| 62) | 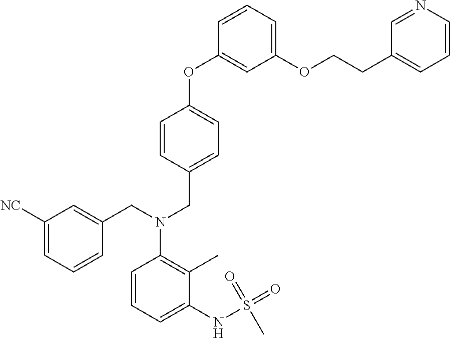 | N-(3-((3- cyanobenzyl)(4-(3- (2-(pyridin-3- yl)ethoxy)phenoxy) benzyl)amino)-2- methylphenyl) methanesulfonamide | (DMSO-d₆, 400 MHz) δ 9.00 (s, 1H), 8.50 (s, 1H), 8.41 (d, J = 8.4 Hz, 1H), 7.71 (d, J = 7.6 Hz, 1H), 7.65-7.57 (m, 3H), 7.47 (t, J = 7.6 Hz, 1H), 7.32-7.20 (m, 4H), 7.03- 6.89 (m, 5H), 7.70-7.67 (dd, J₁ = 8 Hz, J₂ = 1.8 Hz, 1H), 6.50-6.47 (m, 2H), 4.16 (t, J = 6.4 Hz, 2H), 4.12 (s, 2H), 4.02 (s, 2H), 3.01 (t, J = 6.4 Hz, 2H), 2.90 (s, 3H), 2.38 (s, 3H). m/z (M + H)⁺: 619.30 |
| 63) | 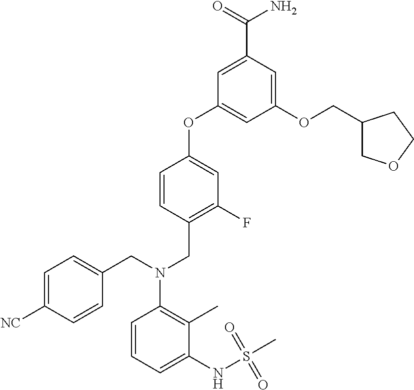 | 3-(4-(((4- cyanobenzyl)(2- methyl-3- (methylsulfonamido) phenyl)amino)methyl)- 3-fluorophenoxy)- 5-((tetrahydrofuran- 3- yl)methoxy)benzamide | (CDCl₃, 400 MHz) δ 7.59 (d, J = 8.4 Hz, 2H), 7.46 (d, J = 8.4 Hz, 2H), 7.24 (s, 1H), 7.16 (t, J = 8.0 Hz, 1H), 7.07 (d, J = 8.0 Hz, 1H), 7.00 (t, J = 1.8 Hz, 1H), 6.90-6.86 (m, 2H), 6.75-6.72 (m, 2H), 6.68- 6.65 (dd, J₁ = 10.4 Hz, J₂ = 2.4 Hz, 1H), 6.62-6.59 (dd, J₁ = 8.4 Hz, J₂ = 2.2 Hz, 1H), 6.05 (s, 1H), 5.79 (s, 1H), 4.18 (s, 2H), 4.02 (s, 2H), 3.97-3.87 (m, 4H), 3.81-3.75 (m, 1H), 3.72-3.68 (m, 1H), 2.93 (s, 3H), 2.74 (m, 1H), 2.23 (s, 3H), 2.14-2.09 (m, 1H), 1.74-1.61 (m, 1H). m/z (M + H)⁺: 658.90 |
| 64) | 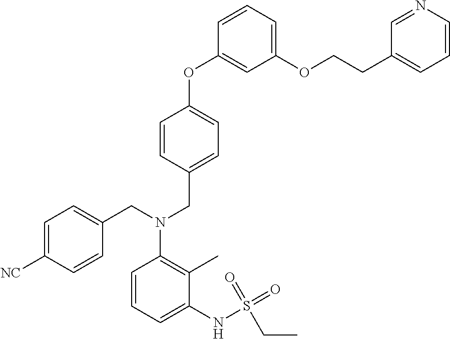 | N-(3-((4- cyanobenzyl)(4-(3- (2-(pyridin-3- yl)ethoxy)phenoxy) benzyl)amino)-2- methylphenyl)ethane sulfonamide | (CDCl₃, 400 MHz) δ 8.52-8.49 (m, 2H), 7.60 (d, J = 8.0 Hz, 1H), 7.56 (d, J = 8 Hz, 1H), 7.33 (d, J = 8.0 Hz, 2H), 7.22-7.18 (m, 4H), 7.12-7.07 (m, 3H), 6.90 (d, J = 8.4 Hz, 2H), 6.85 (d, J = 7.6 Hz, 1H), 6.92-6.60 (m, 1H), 6.58-6.55 (m, 1H), 6.48-6.47 (m, 1H), 6.42 (s, 1H), 4.13 (t, J = 7.4 Hz, 2H), 4.09 (s, 2H), 3.99 (s, 2H), 3.12 (t, J = 7.4 Hz, 2H), 3.07 (t, J = 6.6 Hz, 2H), 2.35 (s, 3H), 1.27 (t, J = 6.6 Hz, 3H). m/z (M + H)⁺: 633.29 |
| 65) | 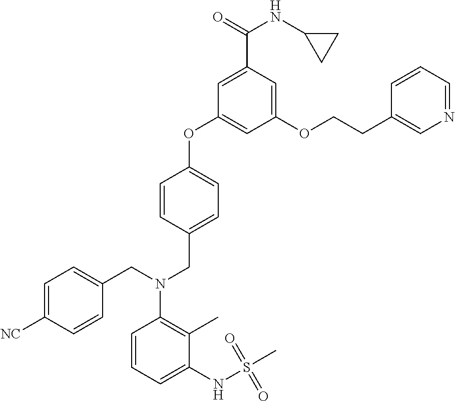 | 3-(4-(((4- cyanobenzyl)(2- methyl-3- (methylsulfonamido) phenyl)amino)methyl) phenoxy)-N- cyclopropyl-5-(2- (pyridin-3- yl)ethoxy)benzamide | (CDCl₃, 400 MHz) δ 8.52 (d, J = 2.0 Hz, 1H), 8.51-8.49 (dd, J₁ = 1.6 Hz, J₂ = 4.8 Hz, 1H), 7.60-7.57 (m, 3H), 7.39 (d, J = 8.0 Hz, 2H), 7.26- 7.20 (m, 2H), 7.14 (t, J = 7.2 Hz, 1H), 7.10-7.07 (m, 2H), 7.03-6.99 (m, 2H), 6.89-6.85 (m, 3H), 6.77- 6.76 (m, 1H), 6.58 (t, J = 2.2 Hz, 1H), 6.15 (s, 1H), 4.18 (t, J = 6.4 Hz, 2H), 4.14 (s, 2H), 3.99 (s, 2H), 3.07 (t, J = 6.4 Hz, 2H), 2.95 (s, 3H), 2.93-2.88 (m, 1H), 2.28 (s, 3H), 1.27-1.20 (m, 2H), 0.88-0.83 (m, 2H). m/z (M + H)⁺: 702.10 |
| 66) | 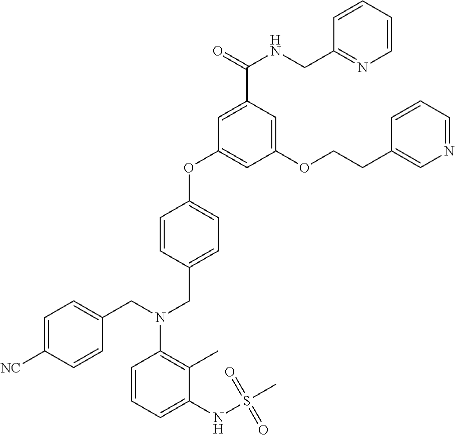 | 3-(4-(((4- cyanobenzyl)(2- methyl-3- (methylsulfonamido) phenyl)amino)methyl) phenoxy)-N- (pyridin-2-ylmethyl)- 5-(2-(pyridin-3- yl)ethoxy)benzamide | (CDCl₃, 400 MHz) δ 8.54-8.50 (m, 3H), 7.68 (dt, J₁ = 7.6 Hz, J₂ = 1.6 Hz, 1H), 7.62-7.58 (m, 4H), 7.54-7.51 (m, 1H), 7.41 (d, J = 8.0 Hz, 2H), 7.33 (d, J = 8 Hz, 1H), 7.23 (d, J = 8.8 Hz, 2H), 7.16 (t, J = 8.0 Hz, 1H), 7.09-7.04 (m, 4H), 6.89-6.85 (m, 3H), 6.62 (t, J = 2.2 Hz, 1H), 4.75 (d, J = 4.8 Hz, 2H), 4.21 (t, J = 6.4 Hz, 2H), 4.15 (s, 2H), 3.99 (s, 2H), 3.09 (t, J = 6.4 Hz, 2H), 2.92 (s, 3H), 2.24 (s, 3H). m/z (M + H)⁺: 753.00 |
| 67) | 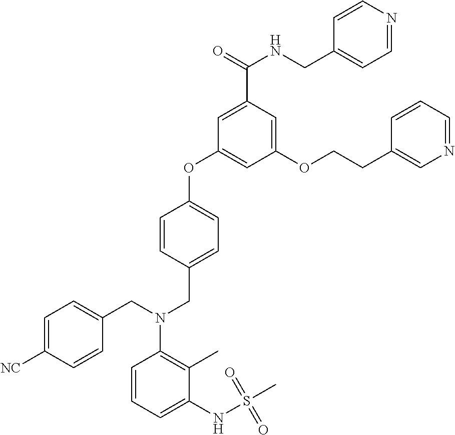 | 3-(4-(((4- cyanobenzyl)(2- methyl-3- (methylsulfonamido) phenyl)amino)methyl) phenoxy)-5-(2- (pyridin-3- yl)ethoxy)-N- (pyridin-4- ylmethyl)benzamide | (CDCl₃, 400 MHz) δ 8.56-8.55 (m, 2H), 8.50-8.49 (m, 2H), 7.60-7.57 (m, 3H), 7.41 (d, J = 2.8 Hz, 2H), 7.24-7.21 (m, 3H), 7.20 (s, 1H), 7.15 (t, J = 8.0 Hz, IH), 7.11 (s, 1H), 7.08-7.03 (m, 3H), 6.98-6.97 (m, 1H), 6.87 (d, J = 8.4 Hz, 2H), 6.83-6.82 (m, 1H), 6.64 (t, J = 2.2 Hz, 1H), 6.59 (t, J = 5.8 Hz, 1H), 4.66 (d, J = 6 Hz, 2H), 4.19 (t, J = 6.2 Hz, 2H), 4.11 (s, 2H), 3.98 (s, 2H), 3.08 (t, J = 6.4 Hz, 2H), 2.88 (s, 3H), 2.22 (s, 3H). m/z (M + H)⁺: 753.00 |
| 68) | 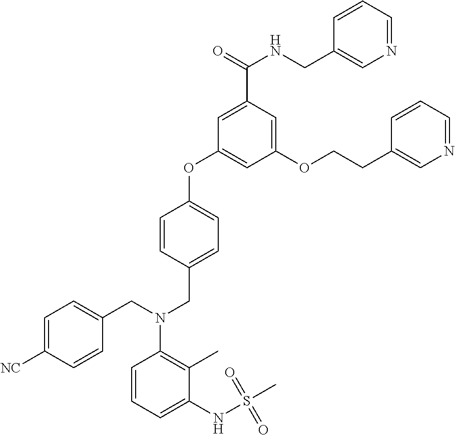 | 3-(4-(((4- cyanobenzyl)(2- methyl-3- (methylsulfonamido) phenyl)amino)methyl) phenoxy)-5-(2- (pyridin-3- yl)ethoxy)-N- (pyridin-3- ylmethyl)benzamide | (CDCl₃, 400 MHz) δ 8.58-8.49 (m, 4H), 7.69 (d, J = 8.0 Hz, 1H), 7.59 (d, J = 8.4 Hz, 3H), 7.41 (d, J = 8.4 Hz, 2H), 7.29-7.20 (m, 3H), 7.16 (d, J = 8.0 Hz, 1H), 7.08-7.03 (m, 3H), 6.94 (t, J = 1.8 Hz, 1H), 6.86 (d, J = 8.4 Hz, 2H), 6.78 (t, J = 1.8 Hz, 1H), 6.62 (t, J = 2.2 Hz, 1H), 6.48 (bt, 1H), 4.65 (d, J = 6.0 Hz, 2H), 4.18 (t, J = 6.4 Hz, 2H), 4.15 (s, 2H), 3.99 (s, 2H), 3.06 (t, J = 6.4 Hz, 2H), 2.90 (s, 3H), 2.24 (s, 3H). m/z (M + H)⁺: 753.30 |
| 69) | 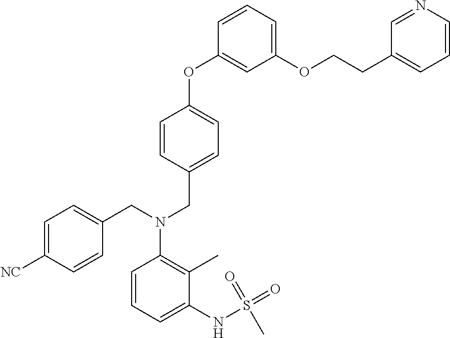 | N-(3-((4- cyanobenzyl)(4-(3- (2-(pyridin-3- yt)ethoxy)phenoxy) benzyl)amino)-2- methylphenyl) methanesulfonamide | (CDCl₃, 400 MHz) δ 8.52-8.59 (m, 2H), 7.62-7.55 (m, 3H), 7.23 (d, J = 8.4 Hz, 2H), 7.26-7.19 (m, 3H), 7.13-7.10 (m, 3H), 6.91-6.87 (m, 3H), 6.63-6.60 (dd, J₁ = 2.0 Hz, J₂ = 8.4 Hz, 1H), 6.58-6.56 (m, 2H), 6.46 (t, J = 2.0 Hz, 1H), 4.15-4.09 (m, 4H), 4.00 (s, 2H), 3.07 (t, J = 6.4 Hz, 2H), 2.98 (s, 3H), 2.36 (s, 3H). m/z (M + H)⁺: 619.30 |
| 70) | 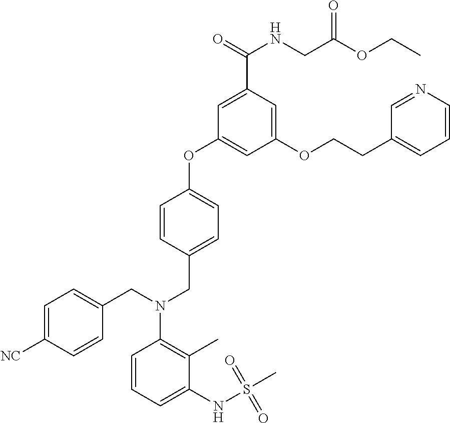 | ethyl 2-(3-(4-(((4- cyanobenzyl)(2- methyl-3- (methylsulfonamido) phenyl)amino)methyl) phenoxy)-5-(2- (pyridin-3- yl)ethoxy)benzamido) acetate | (CDCl₃, 400 MHz) δ 8.53-8.50 (m, 2H), 7.59 (d, J = 8.4 Hz, 3H), 7.41 (d, J = 8.0 Hz, 2H), 7.24-7.17 (m, 2H), 7.13 (d, J = 8.0 Hz, 1H), 7.07 (d, J = 8.4 Hz, 2H), 7.03-6.98 (m, 3H), 6.87 (d, J = 8.8 Hz, 2H), 6.82 (t, J = 1.8 Hz, 1H), 6.63 (t, J = 2 Hz, 1H), 6.59 (t, J = 4.8, 1H), 4.27-4.18 (m, 6H), 4.15 (s, 2H), 3.98 (s, 2H), 3.09 (t, J = 6.0 Hz, 2H), 2.92 (s, 3H), 2.24 (s, 3H), 1.30 (t, J = 7.2 Hz, 3). m/z (M + H)⁺: 748.10 |
| 71) | 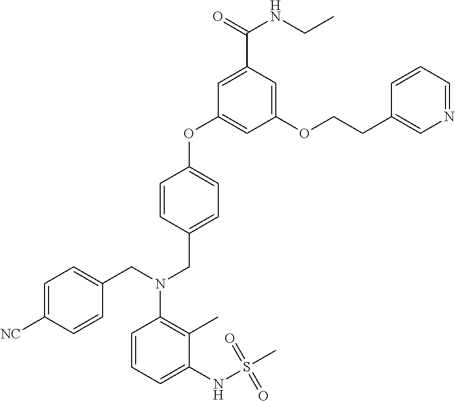 | 3-(4-(((4- cyanobenzyl)(2- methyl-3- (methylsulfonamido) phenyl)amino)methyl) phenoxy)-N-ethyl-5- (2-(pyridin-3- yl)ethoxy)benzamide | (CDCl₃, 400 MHz) δ 8.54 (m, 2H), 7.62-7.57 (m, 3H), 7.40 (d, J = 8.4 Hz, 2H), 7.27-7.21 (m, 2H), 7.15 (t, J = 8 Hz, 1H), 7.09-7.07 (m, 3H), 7.02-6.99 (m, 1H), 6.92-6.91 (m, 1H), 6.88-6.86 (m, 2H), 6.79- 6.78 (m, 1H), 6.59 (t, J = 2.0 Hz, 1H), 6.03 (bt, 1H), 4.19 (t, J = 6.4 Hz, 2H), 4.14 (s, 2H), 3.99 (s, 2H), 3.51-3.44 (m, 2H), 3.08 (t, J = 6.4 Hz, 2H), 2.93 (s, 3H), 2.26 (s, 3H), 1.22 (t, J = 7.2 Hz, 3 H). m/z (M + H)⁺: 690.40 |
| 72) | 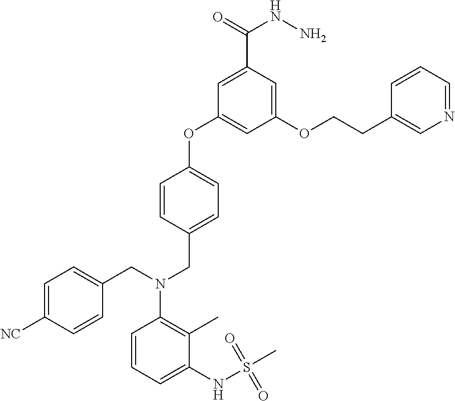 | N-(3-((4- cyanobenzyl)(4-(3- (hydrazinecarbonyl)- 5-(2-(pyridin-3- yl)ethoxy)phenoxy) benzyl)amino)-2- methylphenyl) methanesulfonamide | (CDCl₃, 400 MHz) δ 8.54-8.50 (m, 2H), 7.61-7.58 (m, 3H), 7.45 (d, J = 8.0 Hz, 1H), 7.40 (bs, 1H), 7.35 (s, 1H), 7.31-7.24 (m, 2H), 7.18 (t, J = 8.0 Hz, 1H), 7.14-7.10 (m, 2H), 7.06-7.04 (m, 2H), 6.90-6.85 (m, 3H), 6.66 (t, J = 2.2 Hz, 1H), 6.45 (t, J = 1.8 Hz, 1H), 4.20-4.11 (m, 4H), 3.98 (s, 2H), 3.09 (t, J = 6.4 Hz, 2H), 2.90 (s, 3H), 2.17 (s, 3H). m/z (M + H)⁺: 677.10 |
| 73) | 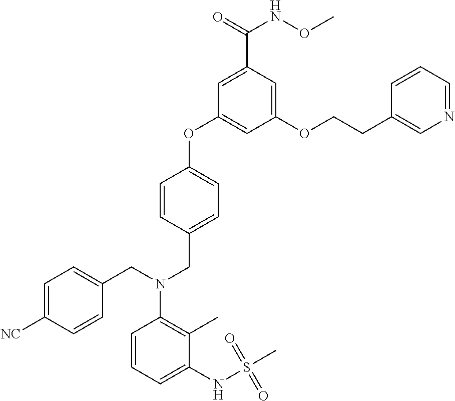 | 3-(4-(((4- cyanobenzyl)(2- methyl-3- (methylsulfonamido) phenyl)amino)methyl) phenoxy)-N- methoxy-5-(2- (pyridin-3- yl)ethoxy)benzamide | (CDCl₃, 400 MHz) δ 8.65-8.57 (m, 1H), 8.53-8.51 (m, 2H), 7.61-7.58 (m, 3H), 7.39 (d, J = 8.0 Hz, 2H), 7.26-7.24 (m, 1H), 7.21 (d, J = 8.0 Hz, 1H), 7.16 (d, J = 8.0 Hz, 1H), 7.11 (d, J = 8.4 Hz, 2H), 7.00-6.94 (m, 2H), 6.88 (d, J = 8.4 Hz, 2H), 6.82-6.79 (m, 2H), 6.61 (s, 1H), 4.19 (t, J = 6.4 Hz, 2H), 4.15 (s, 2H), 4.01 (s, 2H), 3.87 (s, 3H), 3.08 (t, J = 6.4 Hz, 2H), 2.98 (s, 3H), 2.32 (s, 3H). m/z (M + H)⁺: 692.10 |
| 74) | 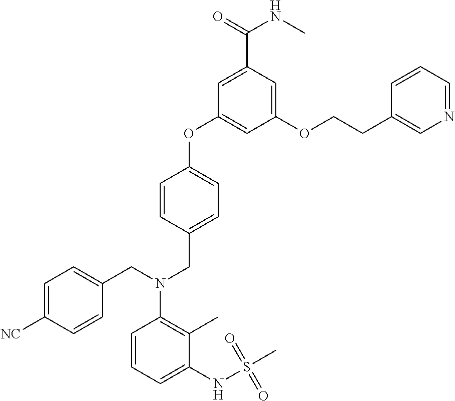 | 3-(4-(((4- cyanobenzyl)(2- methyl-3- (methylsulfonamido) phenyl)amino)methyl) phenoxy)-N-methyl- 5-(2-(pyridin-3- yl)ethoxy)benzamide | (CDCl₃, 400 MHz) δ 8.53-8.50 (m, 2H), 7.62-7.58 (m, 3H), 7.42 (d, J = 8.0 Hz, 2H), 7.24-7.21 (m, 1H), 7.15 (t, J = 8.0 Hz, 1H), 7.09-7.06 (m, 3H), 7.03-6.99 (m, 1H), 6.92 (t, J = 2.0 Hz, IH), 6.87 (d, J = 8.4 Hz, 2H), 6.77 (t, J = 1.6 Hz, 1H), 6.60 (t, J = 2 Hz, 1H), 6.09 (d, J = 4.4 Hz, 1H), 4.18 (t, J = 6.4 Hz, 2H), 4.15 (s, 2H), 3.99 (s, 2H), 3.08 (t, J = 6.0 Hz, 2H), 2.99 (d, J = 4.8 Hz, 3H), 2.94 (s, 3H), 2.26 (s, 3H). m/z (M + H)⁺: 676.10 |
| 75) | 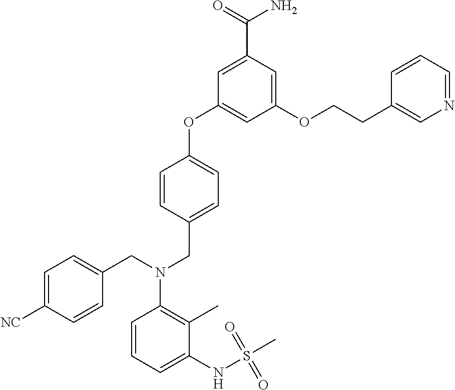 | 3-(4-(((4- cyanobenzyl)(2- methyl-3- (methylsulfonamido) phenyl)amino)methyl) phenoxy)-5-(2- (pyridin-3- yl)ethoxy)benzamide | (CDCl₃, 400 MHz) δ 8.53 (d, J = 1.6 Hz, 1H), 8.51-8.50 (dd, J₁ = 1.2 Hz & J₂ = 4.8 Hz, 1H), 7.61-7.58 (m, 3H), 7.42 (d, J = 8.0 Hz, 2H), 7.26-7.22 (m, 2H), 7.17-7.14 (m, 2H), 7.08-7.03 (m, 3H), 6.96-6.95 (m, 1H), 6.88-6.86 (m, 2H), 6.76- 6.75 (m, 1H), 6.64 (t, J = 2.4 Hz, 1H), 6.01 (bs, 1H), 5.70 (bs, 1H), 4.19 (t, J = 6.4 Hz, 2H), 4.15 (s, 2H), 3.98 (s, 2H), 3.09 (t, J = 6.4 Hz, 2H), 2.94 (s, 3H), 2.25 (s, 3H). m/z (M + H)⁺: 662.10 |
| 76) | 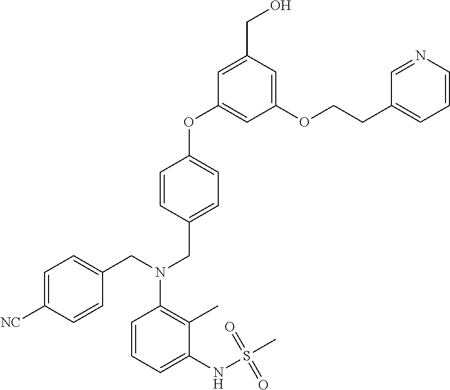 | N-(3-((4- cyanobenzyl)(4-(3- (hydroxymethyl)-5- (2-(pyridin-3- yl)ethoxy)phenoxy) benzyl)amino)-2- methylphenyl) methanesulfonamide | (CDCl₃, 400 MHz) δ 8.51-8.49 (m, 2H), 7.61 (t, 1H), 7.57 (d, J = 8.4 Hz, 2H), 7.37 (d, J = 8.0 Hz, 2H), 7.26-7.20 (m, 3H), 7.13 (t, J = 8.0 Hz, 1H), 7.08 (d, J = 8.8 Hz, 2H), 6.94 (d, J = 8.0 Hz, 1H), 6.88 (d, J = 8.4 Hz, 2H), 6.62-6.61 (m, 2H), 6.52 (s, 1H), 6.39 (t, J = 2.2 Hz, 1H), 4.61 (d, J = 4.4 Hz, 2H), 4.16- 4.13 (m, 4H), 3.99 (s, 2H), 3.06 (t, J = 6.4 Hz, 2H), 2.96 (s, 3H), 2.30 (s, 3H). m/z (M + H)⁺: 649.10 |
| 77) | 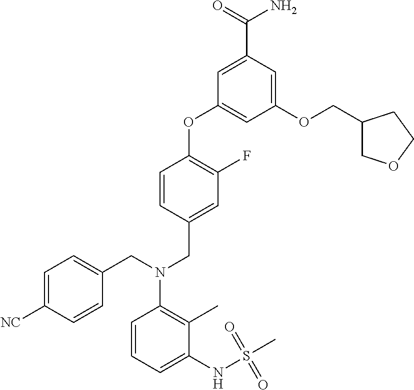 | 3-(4-(((4- cyanobenzyl)(2- methyl-3- (methylsulfonamido) phenyl)amino)methyl)- 2-fluorophenoxy)- 5-((tetrahydrofuran- 3- yl)methoxy)benzamide | (CDCl₃, 400 MHz) δ 7.61 (d, J = 8.4 Hz, 2H), 7.43 (d, J = 8.4 Hz, 2H), 7.18 (t, J = 7.4 Hz, 1H), 7.06 (d, J = 6.8 Hz, 1H), 6.99-6.94 (m, 4H), 6.91-6.88 (dd, J₁ = 10.8 Hz & J₂ = 1.8 Hz, 1H), 6.85 (d, J = 8.4 Hz, 1H), 6.72 (t, J = 2.2 Hz, 1H), 6.68 (s, 1H), 6.00 (bs, 1H), 5.75 (bs, 1H), 4.16 (s, 2H), 3.97 (s, 2H), 3.95-3.87 (m, 4H), 3.81-3.75 (m, 1H), 3.71-3.68 (m, 1H), 2.94 (s, 3H), 2.75-2.71 (m, 1H), 2.24 (s, 3H), 2.12-2.08 (m, 1H), 1.75-1.56 (m, 1H). m/z (M + H)⁺: 658.90 |
| 78) | 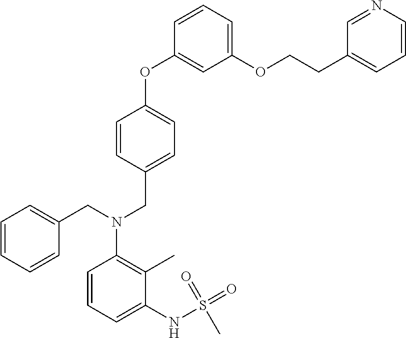 | N-(3-(benzyl(4-(3-(2- (pyridin-3- yl)ethoxy)phenoxy) benzyl)amino)-2- methylphenyl) methanesulfonamide | (CDCl₃, 500 MHz): 7.32-7.23 (m, 5H), 7.18-7.10 (m, 5H), 7.03 (m, 1H), 6.91 (m, 2H), 6.57 (m, 1H), 6.53 (m, 1H), 6.29 (t, 1H, J = 2.4 Hz), 4.07 (s, 2H), 4.03 (s, 2H), 3.43 (s, 6H) and 2.43 (s, 3H). m/z (M + H)⁺: 594.4 |
| 79) | 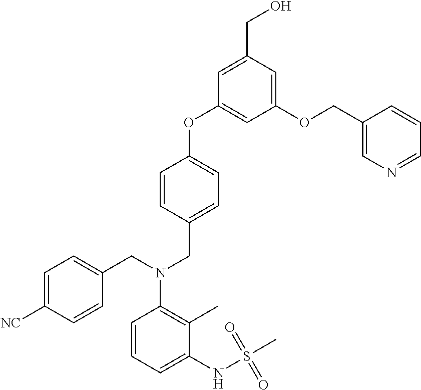 | N-(3-((4- cyanobenzyl)(4-(3- (hydroxymethyl)-5- (pyridin-3- ylmethoxy)phenoxy) benzyl)amino)-2- methylphenyl) methanesulfonamide | (DMSO-D₆, 400 MHz) δ 8.98 (s, 1H), 8.62 (d, J = 1.6 Hz, 1H), 8.53- 8.51 (dd, J₁ = 1.6 Hz & J₂ = 4.8 Hz, 1H), 7.83-7.80 (td, J₁ = 8.0 Hz & J₂ = 1.6 Hz, 1H), 7.72 (d, J = 8.0 Hz, 2H), 7.45 (d, J = 8.0 Hz, 2H), 7.41-7.38 (m, 1H), 7.25 (d, J = 8.4 Hz, 2H), 7.04-7.00 (m, 1H), 6.97- 6.91 (m, 2H), 6.90 (d, J = 8.8 Hz, 2H), 6.74 (s, 1H), 6.49 (d, J = 2.0 Hz, 2H), 5.20 (t, J = 5.6 Hz, 1H), 5.10 (s, 2H), 4.40 (d, J = 5.6 Hz, 2H), 4.15 (s, 2H), 4.02 (s, 2H), 2.91 (s, 3H), 2.38 (s, 3H). m/z (M + H)⁺: 635.00 |
| 80) | 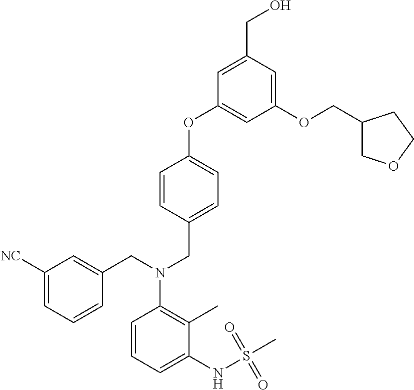 | N-(3-((3- cyanobenzyl)(4-(3- (hydroxymethyl)-5- ((tetrahydrofuran-3- yl)methoxy)phenoxy) benzyl)amino)-2- methylphenyl) methanesulfonamide | (CDCl₃, 400 MHz) δ 7.52-7.51 (m, 2H), 7.45 (d, J = 8.0 Hz, 1H), 7.37 (t, J = 8.0 Hz, 1H), 7.22 (d, J = 7.6 Hz, 1H), 7.14 (t, J = 8.0 Hz, 1H), 7.09 (d, J = 8.4 Hz, 2H), 6.94-6.90 (m, 3H), 6.63 (s, 1H), 6.52 (s, 1H), 6.64 (t, J = 2.0 Hz, 1H), 6.27 (s, 1H), 4.63 (d, J = 4.4 Hz, 2H), 4.11 (s, 2H), 3.98 (s, 2H), 3.91-3.85 (m, 4H), 3.82-3.74 (m, 1H), 3.70-3.64 (m, 1H), 2.97 (s, 3H), 2.75-2.67 (m, 1H), 2.29 (s, 3H), 2.11-2.04 (m, 1H), 1.08 (bt, 1H), 1.75-1.68 (m, 1H). m/z (M + H)⁺: 628.00 |
| 81) | 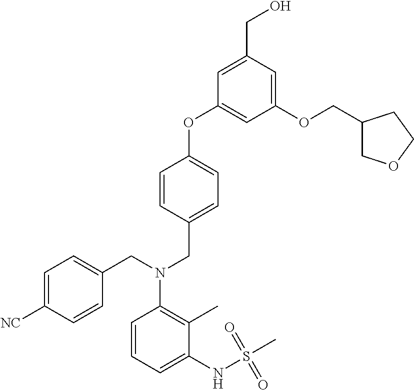 | N-(3-((4- cyanobenzyl)(4-(3- (hydroxymethyl)-5- ((tetrahydrofuran-3- yl)methoxy)phenoxy) benzyl)amino)-2- methylphenyl) methanesulfonamide | (CDCl₃, 400 MHz) δ 7.72 (d, J = 8.0 Hz, 2H), 7.45 (d, J = 8.4 Hz, 2H), 7.26 (d, J = 8.4 Hz, 2H), 6.97- 6.92 (m, 4H), 6.90-6.87 (m, 1H), 6.64 (s, 1H), 6.47 (s, 1H), 6.38 (t, J = 2.4 Hz, 1H), 5.19 (bs, 1H), 4.13 (s, 2H), 4.40 (s, 2H), 4.14 (s, 2H), 4.01 (s, 2H), 3.88-3.80 (m, 2H), 3.77-3.70 (m, 2H), 3.65-3.59 (m, 1H), 3.50-3.46 (m, 1H), 2.85 (s, 3H), 2.63-2.56 (m, 1H), 2.35 (s, 3H), 2.02-1.93 (m, 1H), 1.65-1.22 (m, 1H). m/z (M + H)⁺: 628.20 |
| 82) | 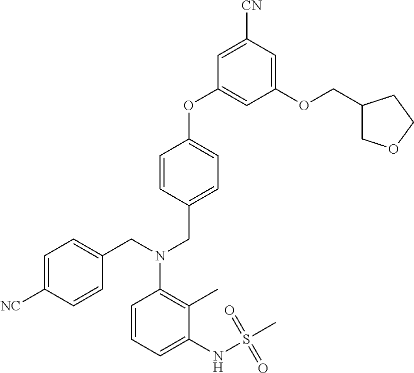 | N-(3-((4-(3-cyano-5- ((tetrahydrofuran-3- yl)methoxy)phenoxy) benzyl)(4- cyanobenzyl)amino)- 2- methylphenyl) methanesulfonamide | (CDCl₃, 400 MHz) δ 7.71 (d, J = 8.0 Hz, 2H), 7.46 (d, J = 8.0 Hz, 2H), 7.32 (d, J = 8.4 Hz, 2H), 7.20 (s, 1H), 7.00-6.98 (m, 3H), 6.92- 6.86 (m, 2H), 6.82 (t, J = 2.0 Hz, 1H), 6.70 (d, J = 6.8 Hz, 1H), 4.13 (s, 2H), 4.01 (s, 2H), 3.98-3.88 (m, 2H), 3.75-3.69 (m, 2H), 3.64-3.56 (m, 1H), 3.37-3.32 (m, 1H), 2.86 (s, 3H), 2.75-2.72 (m, 1H), 2.31 (s, 3H), 2.04-1.92 (m, 1H), 1.63-1.58 (m, 1H). m/z (M + H)⁺: 623.00 |
| 83) | 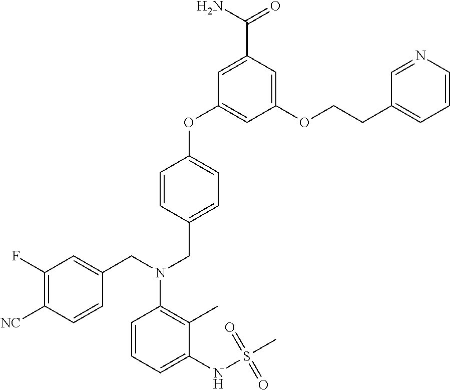 | 3-(4-(((4-cyano-3- fluorobenzyl)(2- methyl-3- (methylsulfonamido) phenyl)amino)methyl) phenoxy)-5-(2- (pyridin-3- yl)ethoxy)benzamide | (CDCl₃, 400 MHz) δ 854-8.55 (m, 2H), 7.63-7.59 (m, 1H), 7.56 (t, J = 7.2 Hz, 1H), 7.27-7.21 (m, 4H), 7.16 (t, J = 8.2 Hz, 1H), 7.07 (d, J = 8.8 Hz, 2H), 7.02 (d, J = 8.0 Hz, 2H), 6.91 (t, J = 2.0 Hz, 1H), 6.88 (d, J = 8.8 Hz, 2H), 6.76 (t, J = 2.0 Hz, 1H), 6.65 (t, J = 2.4 Hz, 1H), 6.00 (bs, 1H), 5.71 (bs, 1H), 4.20 (t, J = 6.4 Hz, 2H), 4.16 (s, 2H), 3.98 (s, 2H), 3.09 (t, J = 6.4 Hz, 2H), 2.96 (s, 3H), 2.65 (s, 3H). m/z (M + H)⁺: 680.00 |
| 84) | 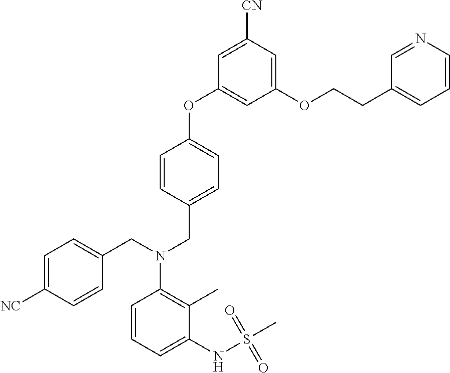 | N-(3-((4-(3-cyano-5- (2-(pyridin-3- yl)ethoxy)phenoxy) benzyl)(4- cyanobenzyl)amino)- 2- methylphenyl) methanesulfonamide | CDCl₃, 400 MHz) δ 8.59-8.51 (m, 2H), 7.60-7.57 (m, 3H), 7.35 (d, J = 8.0 Hz, 2H), 7.32-7.25 (m, 1H), 7.20-7.17 (m, 3H), 7.12 (t, J = 8.0 Hz, 1H), 6.92-6.87 (m, 3H), 6.83- 6.82 (m, 1H), 6.78-6.77 (m, 1H), 6.64-6.63 (m, 2H), 4.16-4.13 (m, 4H), 4.04 (s, 2H), 3.08 (t, J = 6.0 Hz, 2H), 3.00 (s, 3H), 2.39 (s, 3H). m/z (M + H)⁺: 644.20 |
| 85) | 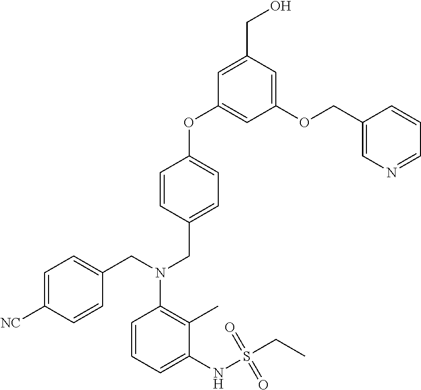 | N-(3-((4- cyanobenzyl)(4-(3- (hydroxymethyl)-5- (pyridin-3- ylmethoxy)phenoxy) benzyl)amino)-2- methylphenyl)ethane sulfonamide | (DMSO-D₆, 400 MHz) δ 8.95 (s, 1H), 8.62 (d, J = 2 Hz, 1H), 8.53- 8.51 (dd, J1 = 1.6 Hz, J2 = 4.8 Hz, 1H), 7.83-7.80 (m, 1H), 7.71 (d, J = 8.4 Hz, 2H), 7.44 (d, J = 8.4 Hz, 2H), 7.41-7.38 (m, 1H), 7.25 (d, J = 8.4 Hz, 2H), 7.01-6.99 (m, 1H), 6.95-6.93 (m, 2H), 6.90 (d, J = 8.4 Hz, 2H), 6.74 (s, 1H), 6.494- 6.490 (m, 2H), 5.19 (t, J = 5.8 Hz, 1H), 5.10 (s, 2H), 4.41 (d, J = 5.8 Hz, 2H), 4.14 (s, 2H), 4.02 (s, 2H), 3.03-2.97 (q, J = 7.6 Hz, 2H), 2.37 (s, 3H), 1.19 (t, J = 7.6 Hz, 3H). m/z (M + H)⁺: 648.90 |
| 86) | 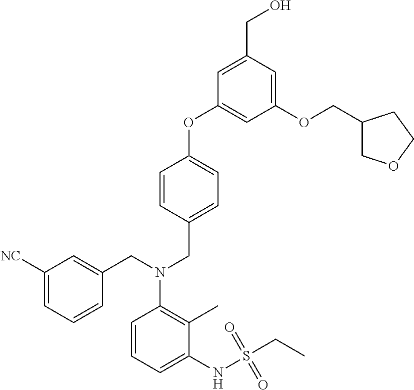 | N-(3-((3- cyanobenzyl)(4-(3- (hydroxymethyl)-5- ((tetrahydrofuran-3- yl)methoxy)phenoxy) benzyl)amino)-2- methylphenyl)ethane sulfonamide | (DMSO-D₆, 400 MHz) δ 8.96 (s, 1H), 7.65-7.63 (m, 1H), 7.61-7.57 (m, 2H), 7.46 (t, J = 7.6 Hz, 1H), 7.26 (d, J = 8.8 Hz, 2H), 7.04-7.00 (m, 1H), 6.96-6.90 (m, 4H), 6.647- 6.645 (m, 1H), 6.46 (s, 1H), 6.37 (t, J = 2.4 Hz, 1H), 5.17 (t, J = 6.0 Hz, 1H), 4.40 (d, J = 6.0 Hz, 2H), 4.12 (s, 2H), 4.02 (s, 2H), 3.90- 3.79 (m, 2H), 3.76-3.69 (m, 2H), 3.65-3.59 (m, 1H), 3.49-3.46 (m, 1H), 3.03-2.97 (q, J = 7.6 Hz, 2H), 2.62-2.55 (m, 1H), 2.37 (s, 3H), 2.01-1.92 (m, 1H), 2.62-1.55 (m, 1H), 1.19 (t, J = 7.6 Hz, 3H). m/z (M + H)⁺: 641.90 |
| 87) | 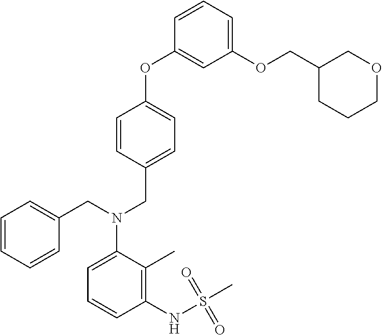 | N-(3-(benzyl(4-(3- ((tetrahydro-2H- pyran-3- yl)methoxy)phenoxy) benzyl)amino)-2- methylphenyl) methanesulfonamide | (CDCl₃, 500 MHz) δ 7.28-7.25 (m, 2H), 7.24-7.18 (m, 5H), 7.15- 7.10 (m, 3H), 6.92-6.89 (m, 3H), 6.62 (m, 1H), 6.54 (m, 1H), 6.51 (t, 1H, J = 2.3 Hz), 4.05 (s, 2H), 4.02 (s, 2H), 3.99 (m, 1H), 3.87-3.75 (m, 3H), 3.46 (m, 1H), 3.34 (dd, 1H, J = 9.1, 11.2 Hz), 2.95 (s, 3H), 2.35 (s, 3H), 2.11 (m, 1H), 1.87 (m, 1H), 1.67-1.62 (m, 2H) and 1.42 (m, 1H). m/z (M + H)⁺: 587.4 |
| 88) | 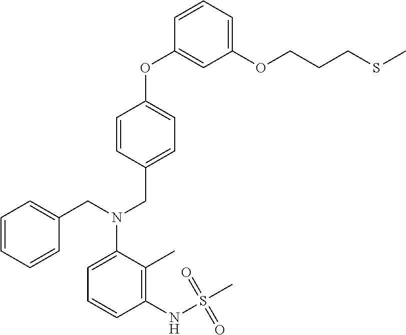 | N-(3-(benzyl(4-(3-(3- (methylthio)propoxy) phenoxy)benzyl) amino)-2- methylphenyl) methanesulfonamide | (CDCl₃, 500 MHz) δ 7.29-7.18 (m, 7H), 7.14 (m, 2H), 7.11 (t, 1H, J = 8.0 Hz), 6.92-6.90 (m, 3H), 6.64 (m, 1H), 6.56-6.53 (m, 2H), 4.05 (s, 2H), 4.02 (t, 2H, J = 6.1 Hz), 4.01 (s, 2H), 2.96 (s, 3H), 2.66 (t, 2H, J = 7.1Hz), 2.33 (s, 3H), 2.11 (s, 3H) and 2.05 (m, 2H). m/z (M + H)⁺: 577.3 |
| 89) | 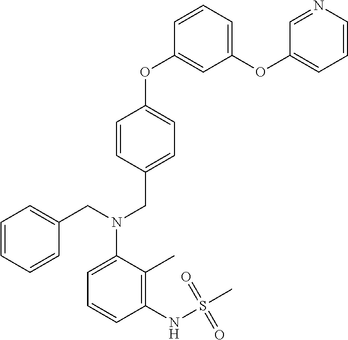 | N-(3-(benzyl(4-(3- (pyridin-3- yloxy)phenoxy)benzyl) amino)-2- methylphenyl) methanesulfonamide | (CDCl₃, 500 MHz) δ 7.44-7.09 (m, 14H), 6.94-6.91 (m, 3H), 6.78 (dd, 1H, J = 8.3, 2.0 Hz), 6.74 (dd, 1H, J = 8.1, 1.9 Hz), 6.63 (t, 1H, J = 2.2 Hz), 4.05 (s, 2H), 4.02 (s, 2H), 2.96 (s, 3H) and 2.33 (s, 3H). m/z (M + H)⁺: 566.2 |
| 90) | 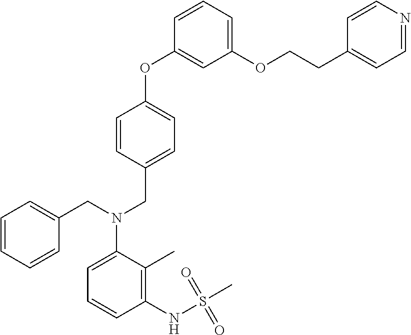 | N-(3-(benzyl(4-(3-(2- (pyridin-4- yl)ethoxy)phenoxy) benzyl)amino)-2- methylphenyl) methanesulfonamide | (CDCl₃, 500 MHz) δ 8.51 (m, 2H), 7.28-7.25 (m, 3H), 7.24-7.18 (m, 6H), 7.14 (m, 2H), 7.11 (t, 1H, J = 7.9 Hz), 6.92-6.89 (m, 3H), 6.62 (m, 1H), 6.56 (m, 1H), 6.51 (t, 1H, J = 2.3 Hz), 4.17 (t, 2H, J = 6.5 Hz), 4.05 (s, 2H), 4.02 (s, 2H), 3.07 (t, 2H, J = 6.5 Hz), 2.95 (s, 3H) and 2.35 (s, 3H). m/z (M + H)⁺: 594.4 |
| 91) | 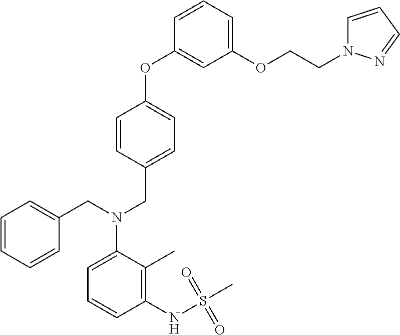 | N-(3-((4-(3-(2-(1H- pyrazol-1- yl)ethoxy)phenoxy) benzyl)(benzyl) amino)-2- methylphenyl) methanesulfonamide | (CDCl₃, 500 MHz) δ 7.52-7.50 (m, 2H), 7.30-7.10 (m, 8H), 6.96-6.91 (m, 2H), 6.88 (m, 2H), 6.60-6.56 (m, 2H9, 6.43-6.40 (m, 2H), 6.24 (t, 1H, J = 2.2 Hz), 4.50 (t, 2H, J = 5.5 Hz), 4.24 (t, 2H, J = 5.5 Hz), 4.06 (s, 2H), 4.02 (s, 2H), 2.95 (s, 3H) and 2.34 (s, 3H). m/z (M + H)⁺: 583.4 |
| 92) | 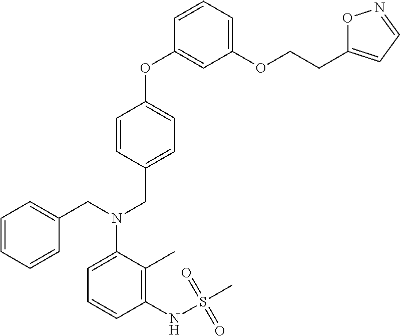 | N-(3-(benzyl(4-(3-(2- (isoxazol-5- yl)ethoxy)phenoxy) benzyl)amino)-2- methylphenyl) methanesulfonamide | (CDCl₃, 500 MHz) δ 8.34 (d, 1H, J = 1.6 Hz), 7.29-7.25 (m, 2H), 7.24- 7.19 (m, 5H), 7.16-7.10 (m, 3H), 6.92-6.89 (m, 3H), 6.64 (m, 1H), 6.57 (m, 1H), 6.52 (t, 1H, J = 2.3 Hz), 6.33 (d, 1H, J = 1.6 Hz), 4.22 (t, 2H, J = 6.4 Hz), 4.05 (s, 2H), 4.02 (s, 2H), 3.19 (t, 2H, J = 6.4 Hz), 2.95 (s, 3H) and 2.34 (s, 3H). m/z (M + H)⁺: 584.3 |
| 93) | 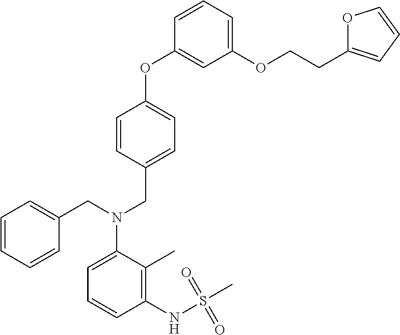 | N-(3-(benzyl(4-(3-(2- (furan-2- yl)ethoxy)phenoxy) benzyl)amino)-2- methylphenyl) methanesulfonamide | (CDCl₃, 500 MHz) δ 7.32 (m, IH), 7.29-7.25 (m, 2H), 7.24-7.19 (m, 4H), 7.15-7.09 (m, 3H), 6.92-6.89 (m, 3H), 6.65 (m, 1H), 6.57-6.53 (m, 2H), 6.30 (m, 1H), 6.12-6.10 (m, 2H), 4.17 (t, 2H, J = 6.9 Hz), 4.05 (s, 2H), 4.02 (s, 2H), 3.11 (t, 2H, J = 6.9 Hz), 2.96 (s, 3H) and 2.35 (s, 3H). m/z (M + H)⁺: 583.3 |
| 94) | 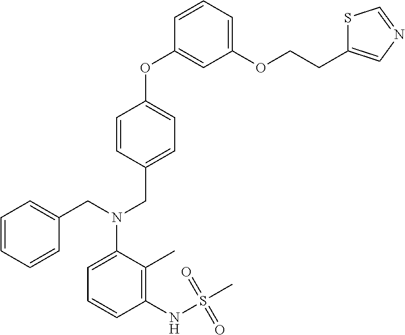 | N-(3-(benzyl(4-(3- (thiazol-5- ylmethoxy)phenoxy) benzyl)amino)-2- methylphenyl) methanesulfonamide | (CDCl₃, 500 MHz) δ 8.81 (s, 1H), 7.85 (s, 1H), 7.29-7.18 (m, 7H), 7.16 (m, 2H), 7.11 (t, 1H, J = 8.1 Hz), 6.92-6.89 (m, 3H), 6.70 (m, 1H), 6.61 (m, 1H), 6.57 (t, 1H, J = 2.3 Hz), 5.24 (s, 2H), 4.05 (s, 2H), 4.02 (s, 2H), 2.95 (s, 3H) and 2.35 (s, 3H). m/z (M + H)⁺: 586.2 |
| 95) | 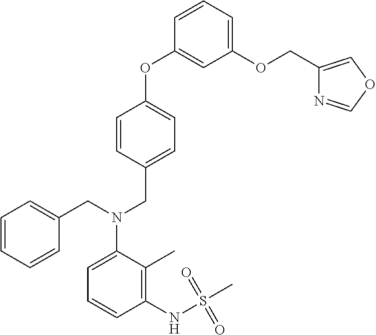 | N-(3-(benzyl(4-(3- (oxazol-4- ylmethoxy)phenoxy) benzyl)amino)-2- methylphenyl) methanesulfonamide | (CDCl₃, 500 MHz) δ 7.90 (s, 1H), 7.70 (m, 1H), 7.29-7.19 (m, 7H), 7.15 (m, 2H), 7.12 (t, 1H, J = 7.1 Hz), 6.93-6.89 (m, 3H), 6.71 (m, 1H), 6.61 (m, 1H), 6.55 (t, 1H, J = 2.4 Hz), 4.97 (s, 2H), 4.06 (s, 2H), 4.02 (s, 2H), 2.94 (s, 3H) and 2.34 (s, 3H). m/z (M + H)⁺: 570.3 |
| 96) | 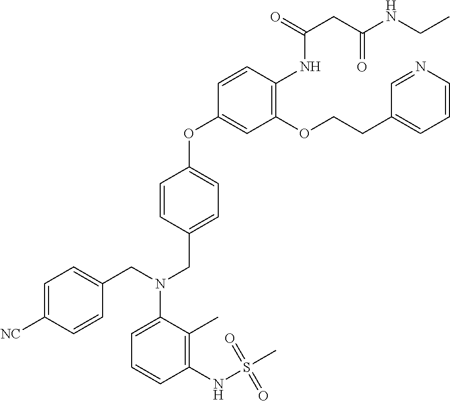 | N1-(4-(4-(((4- cyanobenzyl)(2- methyl-3- (methylsulfonamido) phenyl)amino)methyl) phenoxy)-2-(2- (pyridin-3- yl)ethoxy)phenyl)- N3-ethylmalonamide | (CDCl₃, 500 MHz) δ 8.64 (br s, 1H), 8.51 (br s, 1H), 8.16 (d, 1H, J = 8.6 Hz), 7.79 (m, 1H), 7.55 (m, 2H), 7.33-7.30 (m, 3H), 7.18 (d, 1H, J = 8.4 Hz), 7.11-7.08 (m, 3H), 6.86-6.84 (m, 3H), 6.56-6.53 (m, 2H), 4.17 (t, 2H, J = 6.5 Hz), 4.10 (s, 2H), 3.98 (s, 2H), 3.36 (m, 2H), 3.32 (s, 2H), 3.19 (t, 2H, J = 6.5 Hz), 2.99 (s, 3H), 2.37 (s, 3H) and 1.19 (t, 3H, J = 7.2 Hz). m/z (M + H)⁺: 747.4 |
| 97) | 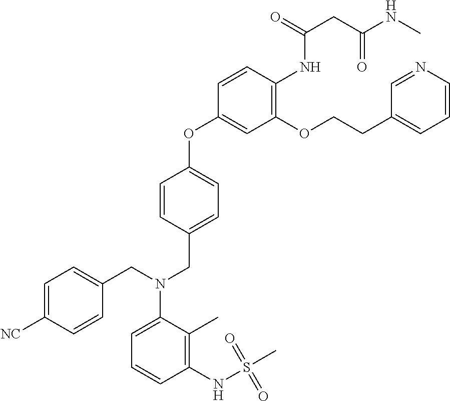 | N1-(4-(4-(((4- cyanobenzyl)(2- methyl-3- (methylsulfonamido) phenyl)amino)methyl) phenoxy)-2-(2- (pyridin-3- yl)ethoxy)phenyl)- N3- methylmalonamide | (CDCl₃, 500 MHz) δ 8.68 (br s, 1H), 8.52 (d, 1H, J = 4.3 Hz), 8.15 (d, 1H, J = 8.7 Hz), 7.83 (m, 1H), 7.55 (m, 2H), 7.37-7.30 (m, 3H), 7.18 (d, 1H, J = 8.2 Hz), 7.11-7.08 (m, 3H), 6.87-6.84 (m, 3H), 6.56- 6.53 (m, 2H), 4.18 (t, 2H, J = 6.1 Hz), 4.10 (s, 2H), 3.98 (s, 2H), 3.35 (s, 2H), 3.21 (t, 2H, J= 6.1 Hz), 2.99 (s, 3H), 2.89 (s, 3H) and 2.36 (s, 3H). m/z (M + H)⁺: 733.4 |
| 98) | 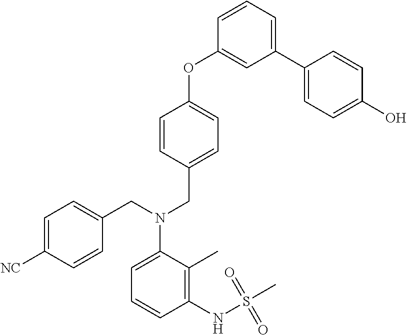 | N-(3-((4- cyanobenzyl)(4-(4′- hydroxybiphenyl-3- yloxy)benzyl)amino)- 2-methylphenyl) methanesulfonamide | (Acetone-d6, 500 MHz) δ 7.67 (m, 2H), 7.53 (m, 2H), 7.48 (m, 2H), 7.40 (t, 1H, J = 8.1 Hz), 7.35 (m, 1H), 7.32 (m, 2H), 7.19-7.16 (m, 2H), 7.08 (t, 1H, J = 8.2 Hz), 7.03 (dd, 1H, J = 8.1, 1.0 Hz), 6.97 (m, 2H), 6.91 (m, 2H), 6.88 (m, 1H), 4.25 (s, 2H9, 4.12 (s, 2H), 2.93 (s, 3H9 and 2.53 (s, 3H). m/z (M + H)⁺: 590.3 |
| 99) | 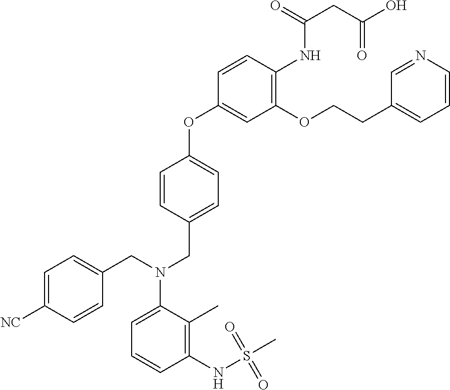 | 3-(3-(4-(((4- cyanobenzyl)(2- methyl-3- (methylsulfonamido) phenyl)amino)methyl) phenoxy)-5-(2- (pyridin-3- yl)ethoxy) phenylamino)-3- oxopropanoic acid | (MeOD, 500 MHz) δ 8.49-8.37 (m, 2H), 7.81 (m, 1H), 7.60 (m, 2H), 7.45 (m, 2H), 7.39 (m, 1H), 7.20 (m, 2H), 7.09-7.05 (m, 2H), 7.00 (m, 1H), 6.96 (s, 1H), 6.88 (m, 2H), 6.80 (s, 1H), 6.23 (s, 1H), 4.19 (s, 2H), 4.17 (t, 2H, J = 6.8 Hz), 4.06 (s, 2H), 3.63 (s, 2H), 3.09 (t, 2H, J = 6.8 Hz), 2.89 (s, 3H) and 2.39 (s, 3H). m/z (M + H)⁺: 720.2 |
| 100) | 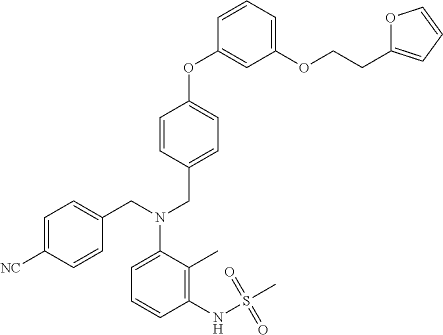 | N-(3-((4- cyanobenzyl)(4-(3- (2-(furan-2- yl)ethoxy)phenoxy) benzyl)amino)-2- methylphenyl) methanesulfonamide | (Acetone-d6, 500 MHz) δ 7.67 (m, 2H), 7.54-7.52 (m, 2H), 7.42 (m, 1H), 7.31 (m, 2H), 7.25 (m, 1H), 7.15 (dd, 1H, J = 7.8, 1.1 Hz), 7.08 (t, 1H, J = 7.9 Hz), 7.02 (dd, 1H, J = 8.0, 1.1 Hz), 6.93 (m, 2H), 6.71 (m, 1H), 6.54-6.52 (m, 2H), 6.32 (dd, 1H, J = 3.1, 1.9 Hz), 6.18 (m, 1H), 4.25 (s, 2H), 4.21 (t, 2H, J = 6.8 Hz), 4.12 (s, 2H), 3.08 (t, 2H, J = 6.8 Hz), 2.93 (s, 3H) and 2.51 (s, 3H). m/z (M + H)⁺: 608.2 |
| 101) | 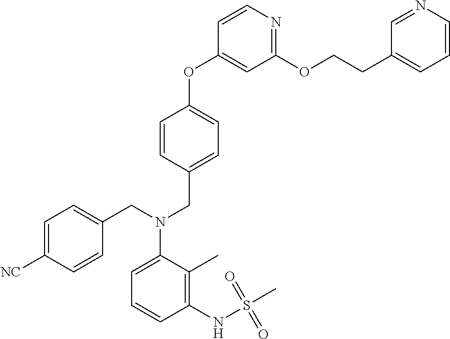 | N-(3-((4- cyanobenzyl)(4-(2- (2-(pyridin-3- yl)ethoxy)pyridin-4- yloxy)benzyl)amino)- 2-methylphenyl) methanesulfonamide | (Acetone-d6, 500 MHz) δ 8.51 (d, 1H, J = 1.7 Hz), 8.43 (dd, 1H, J = 4.9, 1.7Hz), 8.01 (d, 1H, J = 6.0 Hz), 7.72-7.68 (m, 3H), 7.56 (m, 2H), 7.41 (m, 2H), 7.28 (m, 1H), 7.19 (dd, 1H, J = 7.8, 1.3 Hz), 7.10 (t, 1H, J = 7.8 Hz), 7.07-7.04 (m, 3H), 6.54 (dd, 1H, J = 5.9, 2.2 Hz), 6.02 (d, 1H, J = 2.2 Hz), 4.51 (t, 2H, J = 6.8 Hz), 4.28 (s, 2H), 4.18 (s, 2H), 3.06 (t, 1H, J = 6.8 Hz), 2.94 (s, 3H) and 2.53 (s, 3H). m/z (M + H)⁺: 620.2 |
| 102) | 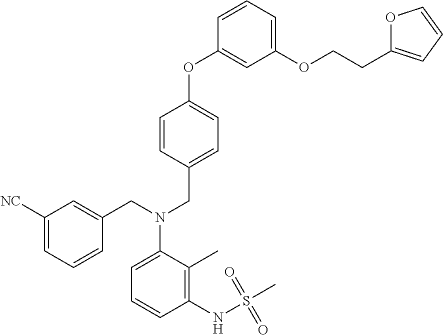 | N-(3-((3- cyanobenzyl)(4-(3- (2-(furan-2- yl)ethoxy)phenoxy) benzyl)amino)-2- methylphenyl) methanesulfonamide | (CDCl₃, 500 MHz) δ 7.52-7.48 (m, 2H), 7.42 (m, 1H), 7.37 (m, 1H), 7.32 (s, 1H), 7.26 (m, 1H), 7.23- 7.20 (m, 2H), 7.14-7.11 (m, 3H), 6.93 (m, 2H), 6.86 (d, 1H, J = 8.0 Hz), 6.66 (dd, 1H, J = 8.1, 2.1Hz), 6.58-6.54 (m, 2H), 6.30 (m, 1H), 6.12 (m, 1H), 4.18 (t, 2H, J = 6.8 Hz), 4.09 (s, 2H), 3.99 (s, 2H), 3.11 (t, 2H, J = 6.8 Hz), 2.99 (s, 3H) and 2.36 (s, 3H). m/z (M + H)⁺: 608.2 |
| 103) | 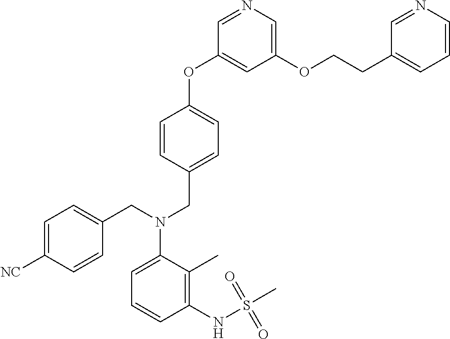 | N-(3-((4- cyanobenzyl)(4-(5- (2-(pyridin-3- yl)ethoxy)pyridin-3- yloxy)benzyl)amino)- 2-methylphenyl) methanesulfonamide | (CDCl₃, 500 MHz) δ 8.53 (br s, 2H), 8.04 (br s, 1H), 7.98 (br s, 1H), 7.60 (m, 1H), 7.58 (m, 2H), 7.35 (m, 2H), 7.20-7.10 (m, 4H), 6.92 (m, 2H),6.88 (d, 1H, J = 7.5 Hz), 6.71 (t, 1H, J = 2.3 Hz), 6.66 (br s, 1H), 4.19 (t, 2H, J = 6.6 Hz), 4.14 (s, 2H), 4.02 (s, 2H), 3.09 (t, 2H, J = 6.6 Hz), 3.00 (s, 3H) and 2.38 (s, 3H). m/z (M + H)⁺: 620.2 |
| 104) | 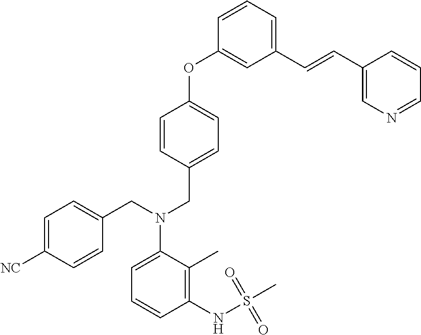 | (E)-N-(3-((4- cyanobenzyl)(4-(3- (2-(pyridin-3- yl)vinyl)phenoxy) benzyl)amino)-2- methylphenyl) methanesulfonamide | (Acetone-d6, 500 MHz) δ 8.76 (d, 1H, J = 1.6 Hz), 8.46 (m, 1H), 8.00 (m, 1H), 7.68 (m, 2H), 7.54 (m, 2H), 7.42-7.26 (m, 8H), 7.17 (d, 1H, J = 7.6 Hz), 7.11-7.04 (m, 2H), 6.96 (m, 2H), 6.91 (m, 1H), 4.26 (s, 2H), 4.14 (s, 2H), 2.94 (s, 3H) and 2.53 (s, 3H). m/z (M + H)⁺: 601.2 |
| 105) | 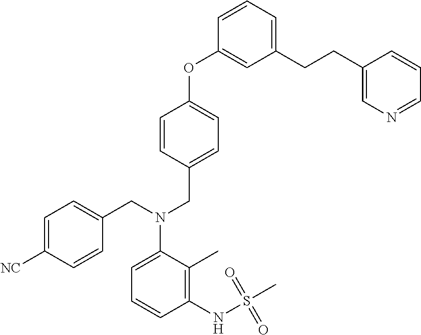 | N-(3-((4- cyanobenzyl)(4-(3- (2-(pyridin-3- yl)ethyl)phenoxy) benzyl)amino)-2- methylphenyl) methanesulfonamide | (Acetone-d6, 500 MHz) δ 8.39- 8.37 (m, 2H), 7.68 (m, 2H), 7.58- 7.53 (m, 3H), 7.30-7.26 (m, 3H), 7.23 (m, 1H), 7.17 (dd, 1H, J = 7.9, 1.2Hz), 7.09 (t, 1H, J = 7.8 Hz), 7.05-7.00 (m, 2H), 6.84 (m, 2H), 6.81-6.79 (m, 2H), 4.26 (s, 2H), 4.12 (s, 2H), 2.94 (s, 3H), 2.93 (s, 4H) and 2.53 (s, 3H). m/z (M + H)⁺: 603.02 |
| 106) | 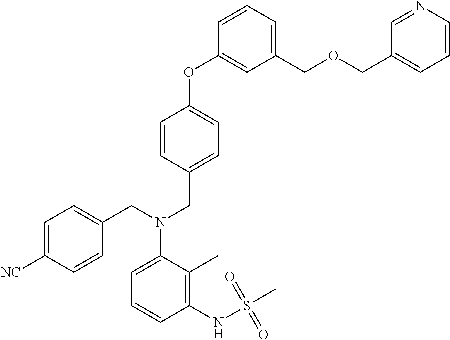 | N-(3-((4- cyanobenzyl)(4-(3- ((pyridin-3- ylmethoxy)methyl) phenoxy)benzyl) amino)-2- methylphenyl) methanesulfonamide | (Acetone-d6, 500 MHz) δ 8.56 (s, 1H), 8.50 (dd, 1H, J = 4.7, 1.1 Hz), 7.75 (m, 1H), 7.67 (m, 2H), 7.54 (m, 2H), 7.38-7.30 (m, 4H), 7.18- 7.14 (m, 2H), 7.09 (t, 1H, J = 8.1 Hz), 7.04-7.02 (m, 2H), 6.94- 6.90 (m, 3H), 4.60 (s, 4H), 4.25 (s, 2H), 4.12 (s, 2H), 2.93 (s, 3H) and 2.52 (s, 3H). m/z (M + H)⁺: 619.2 |
| 107) | 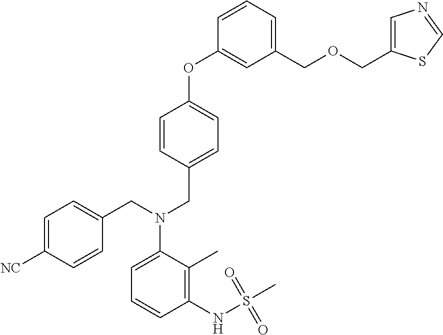 | N-(3-((4- cyanobenzyl)(4-(3- ((thiazol-5- ylmethoxy)methyl) phenoxy)benzyl) amino)-2- methylphenyl) methanesulfonamide | (Acetone-d6, 500 MHz) δ 8.95 (s, 1H), 7.81 (m, 1H), 7.67 (m, 2H), 7.54 (m, 2H), 7.63 (t, 1H, J = 8.0 Hz), 7.32 (m, 2H), 7.17 (dd, 1H, J = 7.8, 1.2 Hz), 7.13-7.03 (m, 3H), 6.99 (m, 1H), 6.93 (m, 2H), 6.91 (m, 1H), 4.82 (s, 2H), 4.57 (s, 2H), 4.26 (s, 2H), 4.13 (s, 2H), 2.93 (s, 3H9 and 2.52 (s, 3H). m/z (M + H)⁺: 625.2 |
| 108) | 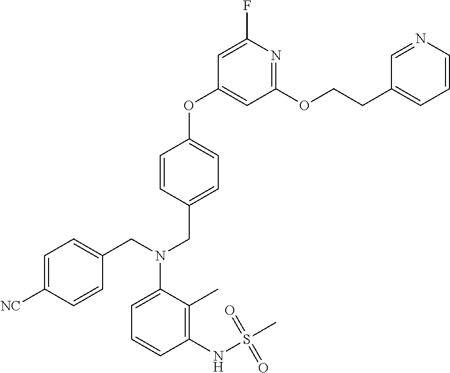 | N-(3-((4- cyanobenzyl)(4-(2- fluoro-6-(2-(pyridin- 3-yl)ethoxy)pyridin-4- yloxy)benzyl)amino)- 2-methylphenyl) methanesulfonamide | (CDCl₃, 500 MHz) δ 8.54 (br s, 2H), 7.75 (d, 1H, J = 8.4 Hz), 7.60 (m, 2H), 7.39 (m, 2H), 7.19 (m, 2H), 7.16-7.12 (m, 2H), 6.97-6.93 (m, 3H), 6.08 (d, 1H, J = 1.6 Hz), 5.81 (d, 1H, J = 1.6 Hz), 4.47 (t, 2H, J = 6.3 Hz), 4.15 (s, 2H), 4.04 (s, 2H), 3.08 (t, 2H, J = 6.3 Hz), 3.00 (s, 3H) and 2.37 (s, 3H). m/z (M + H)⁺: 638.2 |
| 109) | 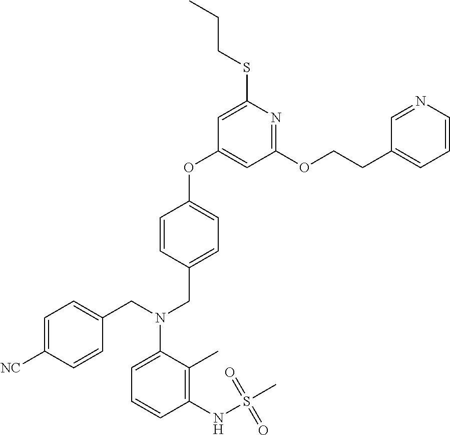 | N-(3-((4- cyanobenzyl)(4-(2- (propylthio)-6-(2- (pyridin-3- yl)ethoxy)pyridin-4- yloxy)benzyl)amino)- 2-methylphenyl) methanesulfonamide | (CDCl₃, 500 MHz) δ 8.59 (br s, 1H), 8.55 (d, 1H, J = 4.9 Hz), 7.59 (m, 2H), 7.48 (m, 1H), 7.39 (m, 2H), 7.22 (m, 1H), 7.18-7.12 (m, 3H), 7.05 (m, 1H), 6.96-6.92 (m, 3H), 6.41 (d, 1H, J = 1.6 Hz), 5.68 (d, 1H, J = 1.9 Hz), 4.54 (t, 2H, J = 6.3 Hz), 4.15 (s, 2H), 4.02 (s, 2H), 3.12 (m, 2H), 3.06 (t, 2H, J = 6.3 Hz), 3.00 (s, 3H), 2.34 (s, 3H), 1.73 (m, 2H) and 1.02 (t, 3H, J = 7.5 Hz). m/z (M + H)⁺: 694.2 |
| 110) | 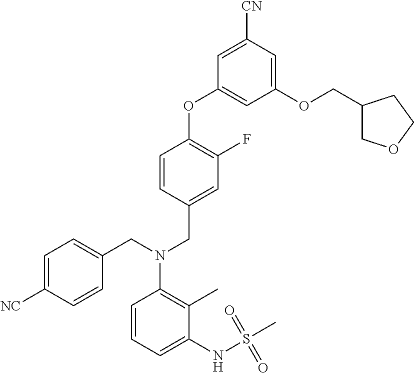 | N-(3-((4-(3-cyano-5- ((tetrahydrofuran-3- yl)methoxy)phenoxy)- 3-fluorobenzyl)(4- cyanobenzyl)amino)- 2-methylphenyl) methanesulfonamide | (CDCl₃, 400 MHz) δ 7.58 (d, J = 8.4 Hz, 2H), 7.32 (d, J = 8 Hz, 2H), 7.22-7.20 (m, 1H), 7.13 (t, J = 8 Hz, 1H), 7.08-7.05 (dd, J₁ = 11.6 Hz & J₂ = 2.0 Hz, IH), 7.03-6.97 (m, 2H), 6.86-6.85 (m, 2H), 6.72 (t, J = 2.4 Hz, 1H), 6.68 (d, J = 1.2 Hz, 1H), 6.18 (bs, 1H), 4.15 (s, 2H), 4.05 (s, 2H), 3.92-3.83 (m, 4H), 3.80-3.74 (m, 1H), 3.70-3.66 (m, 1H), 3.02 (s, 3H), 2.85-2.60 (m, 1H), 2.40 (s, 3H), 2.13-2.09 (m, 1H), 1.72-1.68 (m, 1H), m/z (M + H)⁺: 641.0 |
| 111) | 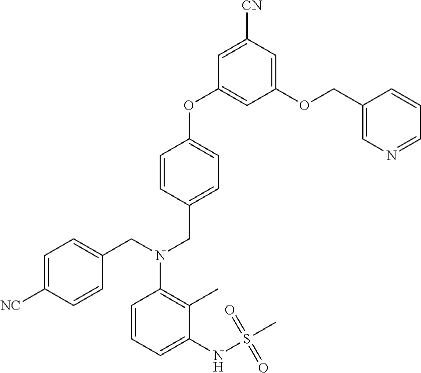 | N-(3-((4-(3-cyano-5- (pyridin-3- ylmethoxy)phenoxy) benzyl)(4- cyanobenzyl)amino)- 2-methylphenyl) methanesulfonamide | (CDCl₃, 400 MHz) δ 8.63-8.61 (m, 2H), 7.74-7.71 (td, J₁ = 8.0 Hz & J₂ = 2.0 Hz, 1H), 7.57 (d, J = 8.4 Hz, 2H), 7.36-7.32 (m, 3H), 7.21-7.18 (m, 3H), 7.11 (t, J = 8.0 Hz, 1H), 6.94-6.92 (m, 3H), 6.86 (d, J = 7.6Hz, 1H), 6.80-6.77 (m, 2H), 6.30 (s, 1H), 5.06 (s, 2H), 4.14 (s, 2H), 4.04 (s, 2H), 3.01 (s, 3H), 2.39 (s, 3H). m/z (M + H)⁺: 630.0 |

The compounds were tested for their efficacy and safety as follows.

### Efficacy & Safety of the Compounds of the Present Invention:

The in vitro and in vivo efficacies of the compounds of the invention may be determined as follows:

### I) GR Binding Assay

Glucocorticoid receptor (GR) binding assay may be performed as described by M. J., Paul-Clark et. al. (M. J., Paul-Clark et. al. J. Immunol. 2003, 171: pp 3245-3252), with minor modifications. Nuclear extracts of human GR (hGR) expressed in insect cells are used in this competition binding assay. A serial dilution of test compounds are added to the hGR in the presence of ³H-Dexamethasone at a fixed concentration in 96 well plates and incubated over night at +4° C. to reach equlibrium. The protein-ligand complex is separated from free ligand by filtration through GF/B filters and MeltiLex, containing the scintillator, is melted on to the filters. Filters are measured in MicroBeta Trilux and an IC50 value is determined for each compound by using a non-linear 4-parameter logistic model.

### II) GRAF Cell Assay

Testing of compounds for agonist or antagonist activity in GR-mediated transactivation in a cell line stably transfected with human glucocorticoid receptor and a glucocorticoid regulated reporter gene construct containing a GRE and alkaline phosphatase as reporter.

Cells are incubated over night in Ham's F12 (w/o phenol red) containing 1% L-glutamine and 2% FCS. The following day the medium is replaced with serum free OptiMEM.

The cells are stimulated with the indicated concentrations of dexamethasone or test compound (dissolved in DMSO, final DMSO concentration 0.1%) for 48 hours. Control cells are treated with 0.1% DMSO t dexamethasone at 5 nM resp 1 μM.

The amount of secreted alkaline phosphatase in the cell supernatants is determined by chemoluminescense.

### III) Modulation of Tyrosine Aminotransferase (TAT) Induction in Rat Hepatoma Cells (Determination of Transactivation)

H4-II-E cells are incubated overnight in 96 well plates in MEM medium containing 10% FCS and 1% non-essential amino acids. On the next day, the medium is replaced with medium containing 1% DCC stripped FCS. The cells are stimulated with the indicated concentrations of dexamethasone or test compound (dissolved in DMSO, final DMSO concentration 0.1%) for 18 hours. Control cells are treated with 0.1% DMSO. After 18 hours, the cells are lysed in buffer by freeze/thawing in two cycles. The TAT activity is measured in a photometric assay using tyrosine and alpha-ketoglutarate as substrates. Dose-response curves are calculated by fitting to a non-linear 4-parameter logistic model, and absolute EC50 (or IC50) values for the determination of dissociation are derived from these curves and the corresponding curve for dexamethasone on the same experimental occasion.

### IV) Evaluation of Bone Side Effects in MG63 Osteocalcin Cell Assay

The MG63 osteosarcoma cells are often used as a model for osteoblasts. The gene encoding osteocalcin contains a negative GRE and the gene is not transcribed in the presence of glucocorticoids. The level of osteocalcin in serum is clinically used to monitor bone formation.

The cells are seeded in 96 well plates and incubated for 72 hours prior to induction.

Cells are washed once and are then induced with medium containing 10 nM of vitD3.

Dexamethasone or test compounds are added at appropriate concentrations (dissolved in DMSO, final concentration 0.1%) and the cells are incubated for 48 hours.

The sectreted concentration of osteocalcin in the cell supernatants are measured by ELISA (e.g. Immunodiagnosticsystems Idsplc, N-MID Osteocalcin One Step ELISA).

Dexamethasone shows an inhibition of vitD3-induced osteocalcin which is an unwanted effect.

The response of dexamethasone is set to 100% and the % efficacy for the test compounds was determined in comparison to dexamethasone. Dose-response curves are calculated by fitting to a non-linear 4-parameter logistic model, and absolute EC50 (or IC50) values for the determination of dissociation are derived from these curves and the corresponding curve for dexamethasone on the same experimental occasion.

### V) Modulation of Tyrosine Aminotransferase (TAT) Induction in Rat Hepatoma Cells (Determination of Transactivation)

H4-II-E-C3 cells are incubated overnight in 96 well plates in MEM medium containing 10% heat inactivated FBS and 1% non-essential amino acids. The following day, cells are stimulated with the indicated concentrations of dexamethasone or test compound (dissolved in DMSO, final DMSO concentration 0.2%) for 18 hours. Control cells are treated with 0.2% DMSO. After 18 hours, the cells are lysed in a buffer containing 0.1% Triton X-100 and the TAT activity is measured in a photometric assay using tyrosine and alpha-ketoglutarate as substrates. TAT activity of Dexamethasone at 10 nM concentration was considered as 100% and % efficacy was determined as the TAT activity at 25 μM concentration of the test NCE. Dose-response curves are calculated by fitting to a non-linear 4-parameter logistic model, and absolute EC50 (or IC50) values for the determination of dissociation are derived from these curves and the corresponding curve for dexamethasone on the same experimental occasion.

### VI) Modulation of Proinflammatory Cytokine Production in U 937 Cells (Determination of Transrepression)

Testing of compounds in GR-mediated inhibition of LPS-induced TNFα, IL-1β, and IL-6 secretion in U 937 cells.

Differentiated U 937 cells are incubated for 2 days in RPMI1640 medium containing 10% CCS (charcoal treated calf serum). The cells are transferred to 96 well plates and stimulated with 5 μg/mL LPS (dissolved in PBS) in the presence or absence of dexamethasone or test compound (dissolved in DMSO, final concentration 0.2%). Control cells are treated with 0.2% DMSO. After required incubation periods, proinflammatory cytokine(s) concentrations in the cell supernatant are measured by ELISA, using the “BD OptEIA™ Set human TNF, BD OptEIA™ Set human IL-6, BD OptEIA™ Set human IL-1β.

TNF α inhibition induced by Dexamethasone at 10 nM concentration was considered as 100% and % inhibition of the test NCE was determined as compared to Dexamethasone. Dose-response curves are calculated by fitting to a non-linear 4-parameter logistic model, and absolute EC50 (or IC50) values for the determination of dissociation are derived from these curves and the corresponding curve for dexamethasone on the same experimental occasion. Data from this assay is depicted below:

| | |
|---|---|
| % IL 6 Inhibition | % TNF α Inhibition |

| Example. No. | 0.1 μM | 1 μM | 0.1 μM | 1 μM |
|---|---|---|---|---|
| 16 | 62 | 81 | 63.17 | 87.42 |
| 20 | 92.2 | 97.9 | 88.89 | 93.63 |
| 35 | 28 | 54 | 41.76 | 83.43 |
| 55 | 55 | 76 | 1.54 | −1.12 |
| 58 | 45 | 76 | 13.14 | 55.04 |
| 60 | 90.0 | 91.7 | 95.1 | 96.3 |
| 62 | 68.4 | 90.8 | 52.56 | 83.68 |
| 74 | 100 | 100 | 90.4 | 100 |
| 75 | 98.2 | 100 | 75.3 | 100 |
| 76 | 85.7 | 88.8 | 87.6 | 86.2 |
| 78 | 62 | 74 | 67.86 | 83.86 |
| 79 | 97 | 97 | 87.7 | 88.2 |
| 80 | ND | ND | 84.1 | 86.5 |
| 81 | ND | ND | 88.9 | 88.2 |
| 82 | 60.0 | 83.0 | 50.0 | 71.1 |
| 83 | 95.7 | 95.1 | 93.4 | 94.5 |
| 84 | 95.0 | 94.9 | 94.6 | 93.7 |
| 85 | ND | ND | 88.5 | 91.2 |
| 86 | ND | ND | 95.5 | 95.6 |

ND = Not done

### In Vivo Efficacy Models:

Transrepression may be determined as follows:

### VII) Evaluation of Antiinflammatory Effect in Rat Paw Edema (RPE) Model

Male Wistar rats, 150-180 g body weight, are divided into groups of 8-10 animals each and kept overnight fasted for at least 16 h. Control animals receives vehicle alone p.o. Animals in the other groups are dosed orally (test drug and standard) one hour prior to the subplanter injection of 1% w/v (0.1 ml/rat) carrageenan solution. Paw volumes are measured 0, 3 and 5 hours of carrageenan injection. Inhibition of paw volume at different time points are compared verses the control group. Transactivation may be determined as follows:

### VIII) Blood Glucose and Liver Tyrosine Amino Transferase (TAT) Estimation

Female Wistar rats, 140-160 g body weight, are divided into groups of 8-10 animals each and kept overnight fasted for at least 16 h. They are treated by intraperitoneal administration with vehicle (5% DMSO, 10% Tween 80), dexamethasone (0.3 mg/kg) or test compounds formulated in the vehicle. After 6 h, the animals are bled by retroorbital puncture under ether anesthesia and then sacrificed to collect the liver biopsies which are flash frozen in liquid nitrogen and stored for later analysis. Glucose in plasma is measured by colorimetric assay using a GOD/POD kit from Ranbaxy, N Delhi, India.

Biopsies are homogenized in 2 ml of homogenizing buffer (140 mM KCl in 20 mM potassium phosphate buffer, pH 7.6) and centrifuged to clear out the large debris. Supernatants are assayed for protein content by Protein Assay Kit (Pointe Scientific Inc, Canton, Mich.). Twenty microliters of supernatant is diluted and is incubated at 37° C. for 30 min with 200 μl of TAT-reaction buffer. To stop the incubation, KOH is added to the reaction mixture and extinction is measured at 340 nm. TAT activity is calculated after normalizing against total protein content. TAT induction is defined as x-fold increase in TAT activity compared to the vehicle-treated animals.

### IX) Serum Bone Marker Study

Male Wistar rats are allowed free access to food and water. Animals are weighed and randomly allocated into study groups to receive either vehicle (distilled water containing 5% DMSO, 10% Tween 80), test compound or dexamethasone (0.3, 1 and 3 mg/kg/day). Compounds are prepared as a suspension in vehicle and intraperitoneally administered to rats once daily for 7 days. Body weight is measured daily throughout the study. Twenty four hours after administration of the last dose of compound, blood is collected into ice-cold tubes by retroorbital route, and the serum is stored at 22° C. until analyzed. Following the bleeding, rats are euthenized and thymus, adrenal and spleen is dissected free of connective tissue and weighed. Serum samples are later analyzed for bone related biomarkers, such as osteocalcin and the bone-specific tartrate-resistant ACP (TRAP).

1. A compound of formula (1), 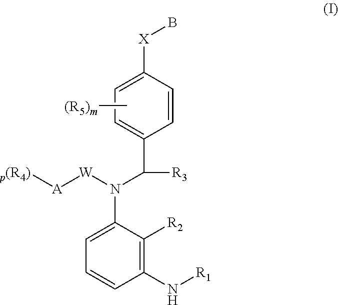
wherein, R₁ represents hydrogen or optionally substituted groups selected from linear or branched —(CH₂)rSO₂R₇, —(CH₂)r—C(O)R₇, —(CH₂)r—C(O)OR₇, —(CH₂)r—C(O)NR₇R₈, —(CH₂)r—C(O)NHOR₇, (CH₂)rSO₂—(CH₂)q—C(O)OR₇, (CH₂)rSO₂—(CH₂)q—OR₇ and SO₂—(CH₂)r—C(O)—NR₇R₈;
r and q independently represent an integer from 0-5;
R₇ and R₈ are the same or different and independently represent hydrogen, hydroxy, or optionally substituted groups selected from (C₁-C₆)alkyl, (C₂-C₄)alkenyl, and (C₂-C₄)alkynyl;
R₂ represents hydrogen, halogen, cyano, (CH₂)m-OR₇, or optionally substituted (C₁-C₆)alkyl;
R₃ represents hydrogen, cyano or optionally substituted groups selected from (C₁-C₆)alkyl and (CH₂)m-O—R₇;
W represents —(CR₇R₈)k-;
k represents an integer from 0-4;
A represents hydrogen, optionally substituted (C₁-C₄)alkyl, phenyl or a heteroaromatic ring;
R₄ at each occurrence independently represents halogen, (C₁-C₄)alkylene-hydroxy, nitro, cyano, CONH₂, —(C₁-C₆)alkyl-OC(O)R₇ or optionally substituted groups selected from (C₁-C₆)alkyl, (C₁-C₆)alkoxy and amino;
p represents an integer from 0-5;
R₅ at each occurrence is independently selected from hydrogen, halogen, hydroxy, cyano and optionally substituted groups selected from linear or branched (C₁-C₆)alkyl and (C₁-C₆)alkoxy;
m represents an integer from 0-4;
X represents —CH₂—, O or S;
B represents a cyclic group selected from phenyl and a heteroaromatic ring, wherein said cyclic group is substituted with a group La, and said cyclic group being optionally further substituted with one or more groups independently selected from R₆ and Lb;
La represents (CH₂)q′-Z, (CH₂)r′-O—(CH₂)q′-Z or CH═CH—Z;
r′ and q′ independently represents an integer from 0-4;
Z represents optionally substituted groups selected from (C₁-C₆)alkyl, (C₂-C₆)alkenyl, (C₂-C₆)alkynyl, (C₃-C₇)cycloalkyl, (C₁-C₆)alkoxy, —OSO₂R₇, —S(C₁-C₆)alkyl, phenyl, or a saturated heterocyclic group or a heteroaromatic ring, said saturated heterocyclic group or heteroaromatic ring optionally being substituted with one or more halogen, cyano or (C₁-C₆)alkyl;
R₆ at each occurrence independently represents halogen, hydroxy, cyano, —S(C₁-C₄)alkyl, or optionally substituted groups selected from linear or branched (C₁-C₆)alkyl and (C₁-C₆)alkoxy;
Lb is selected from T-(CH₂)r-D, wherein T is optionally present and independently represents O, S, —CH₂—, —NH—, —NHCO—, —CONH—, —SO₂NH—, or —NHSO₂—, and r represents an integer from 0-5;
D represents hydroxy, NHSO₂Rc, NHCORc, CONH₂, CONHRc, CONRdRe, SO₂NH₂, SO₃H, COOH, COORc, C(NH)NHOH, C(O)NHOH, C(O)NHORc, C(O)NHNH₂, NHC(O)NH₂ or a heteroaromatic ring;
Rc represents optionally substituted (C₁-C₃)alkyl or (C₃-C₇)cycloalkyl;
Rd and Re independently represent hydrogen, optionally substituted (C₁-C₃)alkyl, —O(C₁-C₃)alkyl or Rd and Re together form a —(CH₂)₄— group or a —(CH₂)₂—O—(CH₂)₂— group;
provided that when p is 0; then X is O and B is phenyl substituted with a group La, wherein La represents (CH₂)r′-O—(CH₂)q′-Z, r′ is 0 and q′ represents an integer from 0-3, and Z represents —S-methyl, an unsubstituted saturated heterocyclic group or a heteroaromatic ring;
or a pharmaceutically acceptable salt thereof.

2. A compound as claimed in claim 1, wherein
R₁ represents —(CH₂)rSO₂R₇;
r represents 0;
R₇ represents optionally substituted (C₁-C₃)alkyl;
R₂ represents hydrogen or methyl;
R₃ represents hydrogen;
W represents —(CR₇R₈)k, wherein R₇ and R₈ represent hydrogen and k is 1;
A represents phenyl or pyridyl;
R₄ independently represents halogen, (C₁-C₄)alkylene-hydroxy, cyano, CONH₂, —(C₁-C₆)alkyl-OC(O)R₇ or optionally substituted (C₁-C₆)alkyl;
p represents an integer from 0-2;
R₅ represents hydrogen, halogen;
m represents 0 or 1;
X represents O;
B represents a cyclic group selected from phenyl and pyridyl, wherein said cyclic group is substituted with one La group, and said cyclic group being optionally further substituted with one R₆ group or one Lb group;
La represents (CH₂)q′-Z, (CH₂)r′-O—(CH₂)q′-Z or CH═CH—Z;
r′ and q′ independently represent an integer from 0-4;
Z represents —S(C₁-C₆)alkyl, phenyl optionally substituted with one hydroxy, or a saturated heterocyclic group or a heteroaromatic ring, said saturated heterocyclic group or heteroaromatic ring optionally being substituted with one (C₁-C₆)alkyl;
R₆ represents halogen, cyano, —S(C₁-C₄)alkyl, or optionally substituted (C₁-C₄)alkyl;
Lb is selected from T-(CH₂)r-D, wherein T is optionally present and independently represents —NHCO— or —CONH—, and r represents 0, 1 or 2;
D represents hydroxy, CONH₂, CONHRc, CONRdRe, COOH, COORc, C(O)NHORc, C(O)NHNH₂, or pyridyl;
Rc represents optionally substituted (C₁-C₃)alkyl or cyclopropyl;
Rd and Re independently represent hydrogen, optionally substituted (C₁-C₃)alkyl, —O(C₁-C₃)alkyl or Rd and Re together form a —(CH₂)₄— group or a —(CH₂)₂—O—(CH₂)₂— group;
provided that when p is 0; then B is selected from 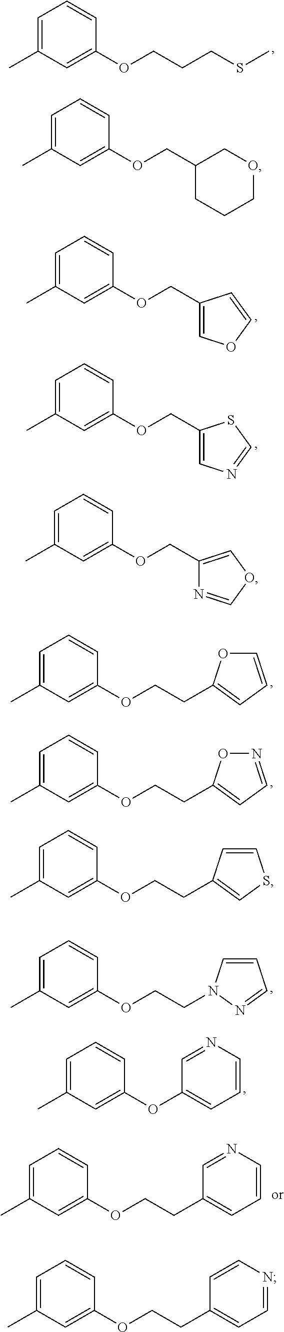
or a pharmaceutically acceptable salt thereof.

3. A compound as claimed in claim 2, wherein
B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re together form a —(CH₂)₄— group or a —(CH₂)₂—O—(CH₂)₂— group, provided that when La is 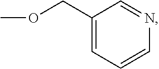
then the Lb group is not CONH₂;
phenyl or pyridyl substituted with one La group only, wherein the La group is selected from one of the following groups: 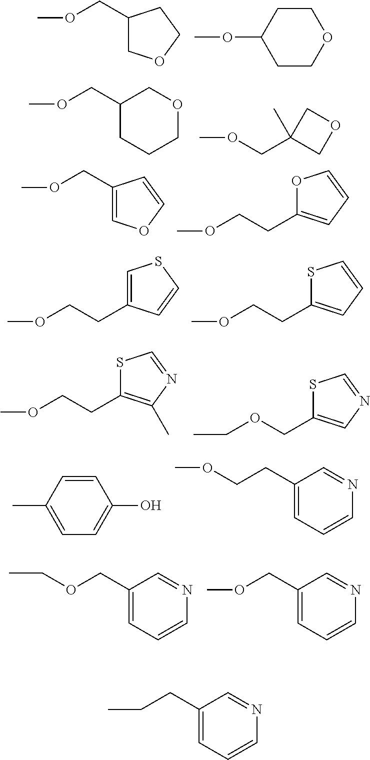
provided that
when La is 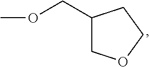
and R₇ is optionally substituted methyl,
then either p represents 1 and R₄ is a methyl, trifluoromethyl or cyano group which is attached at the 4-position of the phenyl or pyridyl A ring relative to group W, or p represents 2 and one of the two R₄ groups is attached at the 4-position of the phenyl or pyridyl A ring relative to group W;
when La is 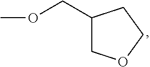
and R₇ is optionally substituted (C₂-C₃)alkyl,
then p represents 1 and R₄ is a cyano group which is attached at the 3-position of the phenyl or pyridyl A ring relative to group W;
when La is 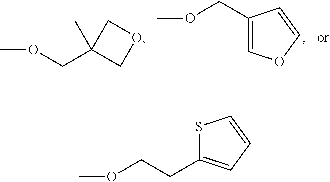
then p represents 1 and R₄ is a cyano group which is attached at the 4-position of the phenyl or pyridyl A ring relative to group W;
when La is 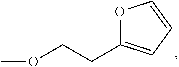
then either p represents 0, or p represents 1 and R₄ is a cyano group which is attached at the 4-position of the phenyl or pyridyl A ring relative to group W; and
when La is 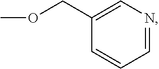
then either R₇ is methyl, p represents 1 and R₄ is a cyano group which is attached at the 4-position of the phenyl or pyridyl A ring relative to group W, or R₇ is ethyl, p represents 1 and R₄ is a cyano group which is attached at the 3-position of the phenyl or pyridyl A ring relative to group W.

4. A compound as claimed in claim 2, wherein
R₂ represents methyl;
W represents —(CR₇R₈)k, wherein R₇ and R₈ represent hydrogen and k is 1;
B is selected from one of the following:
phenyl substituted with one La group, said phenyl being further substituted with one R₅ group or one Lb group, wherein the R₆ group represents halogen, —S(C₁-C₄)alkyl, or optionally substituted (C₁-C₄)alkyl, and wherein the Lb group is selected from T-(CH₂)r-D, wherein T is optionally present and represents —CONH—; D represents hydroxy, CONH₂, CONHRc, C(O)NHORc, C(O)NHNH₂, or pyridyl, wherein Rc represents optionally substituted (C₁-C₃)alkyl or cyclopropyl, provided that
when La is 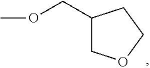
then the phenyl is further substituted one Lb group and the Lb group represents —(CH₂)r-D, wherein D represents CONH₂ or CONHRc;
phenyl substituted with one La group only, wherein the La group is selected from one of the following groups: 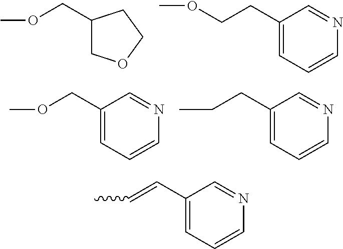
provided that
when La is 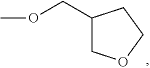
then R₇ is methyl, p represents 1, and R₄ is either a cyano group which is attached at the 3-position or the 4-position of the phenyl A ring relative to group W, or a methyl group which is attached at the 4-position of the phenyl A ring relative to group W;
when La is 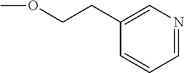
then R₇ represents unsubstituted (C₁-C₃)alkyl, and either p represents 0, or p represents 1 and R₄ is halogen, cyano or methyl; and
when La is 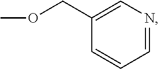
then either R₇ is methyl, p represents 1 and R₄ is a cyano; or R₇ is ethyl, p represents 1 and R₄ is a cyano group which is attached at the 4-position of the phenyl or A ring relative to group W; or
pyridyl substituted with one La group only, wherein the La group represents (CH₂)₂—Z, wherein Z represents pyridyl.

5. A compound as claimed in claim 2, wherein
R₂ represents methyl; and
B is selected from one of the following:
phenyl substituted with one La group, said phenyl being further substituted with one R₆ group or one Lb group, wherein the R₆ group represents cyano, halogen, or —S(C₁-C₄)alkyl, and wherein the Lb group is T-(CH₂)r-D, in which either:
T is absent, r represents 0 or 1, and D represents hydroxy, CONH₂, CONHRc, CONRdRe, C(O)NHNH₂, wherein Rc represents optionally substituted (C₁-C₃)alkyl or cyclopropyl, and Rd and Re independently represent optionally substituted (C₁-C₃)alkyl; or
T is present and represents —CONH—, r represents 1 or 2, and D represents COOH or COORc, wherein Rc represents optionally substituted (C₁-C₃)alkyl; or
T is present and represents r represents 1 or 2, and D represents COOH or COORc, wherein Rc represents optionally substituted (C₁-C₃)alkyl; or;
phenyl substituted with one La group only, wherein the La group is selected from one of the following groups: 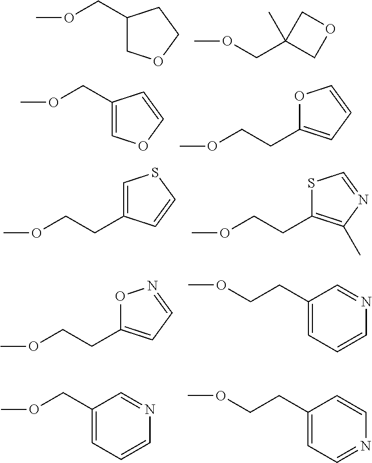
provided that
when La is 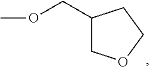
then either R₇ is ethyl, p is 0, 1 or 2, and R₄ is halogen, cyano, CONH₂, or optionally substituted (C₁-C₄)alkyl; or
R₇ is methyl, p represents 1, and either R₄ is a methyl or halogen group which is attached at the 3-position of the phenyl A ring relative to group W, or R₄ is a methyl, cyano or halogen group which is attached at the 4-position of the phenyl
A ring relative to group W; or
R₇ is methyl, p represents 2, R₄ is halogen, cyano, or methyl, and one of the two R₄ groups is attached at the 3-position of the phenyl A ring relative to group W;
when La is 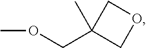
then R₄ is a halogen; and
when La is 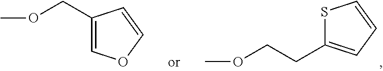
then R₇ is methyl, p represents 1, and R₄ is a cyano group which is attached at the 4-position of the phenyl A ring relative to group W; and
when La is 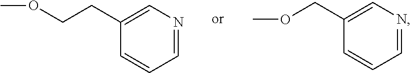
then R₇ represents unsubstituted (C₁-C₃)alkyl, and either p represents 0, or p represents 1 and R₄ is cyano, CONH₂, or methyl.

6. A compound as claimed in claim 2, wherein
B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re together form a —(CH₂)₄— group or a —(CH₂)₂—O—(CH₂)₂— group;
phenyl or pyridyl substituted with one La group only, wherein the La group is selected from one of the following groups: 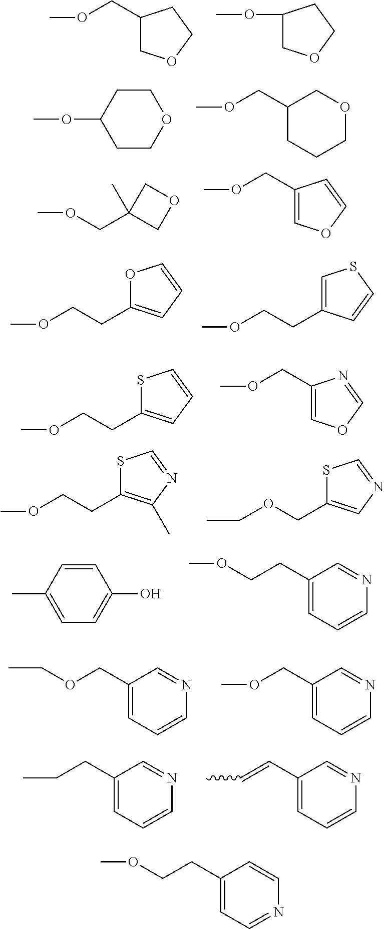
provided that
when La is 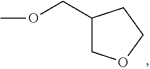
and R₇ is optionally substituted methyl,
then either p represents 1 and R₄ is selected from a cyano group which is attached at the 3-position or the 4-position of the phenyl or pyridyl A ring relative to group W, or a methyl or trifluoromethyl which is attached at the 4-position of the phenyl or pyridyl A ring relative to group W; or p represents 2 and one of the two R₄ groups is attached at the 4-position of the phenyl or pyridyl A ring relative to group W;
when La is 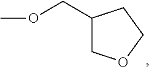
and R₇ is optionally substituted (C₂-C₃)alkyl,
then p represents 1 and R₄ is a cyano group which is attached at the 3-position of the phenyl or pyridyl A ring relative to group W;
when La is 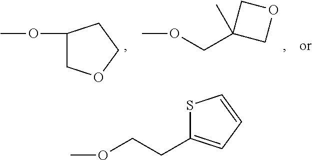
then p represents 1 and R₄ is a cyano group which is attached at the 4-position of the phenyl or pyridyl A ring relative to group W;
when La is 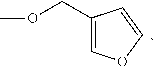
then either R₇ is methyl, p represents 1 and R₄ is a cyano group which is attached at the 4-position of the phenyl or pyridyl A ring relative to group W; or R₇ is ethyl, p represents 1 and R₄ is a cyano group;
when La is 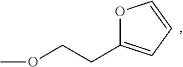
then either p represents 0, or p represents 1 and R₄ is a cyano group which is attached at the 4-position of the phenyl or pyridyl A ring relative to group W;
when La is 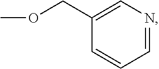
then R₇ represents unsubstituted (C₁-C₃)alkyl, and either p represents 0, or p represents 1 and R₄ is cyano, CONH₂, or methyl.

7. A compound as claimed in claim 2, wherein
R₂ represents methyl;
B is selected from one of the following:
phenyl substituted with one La group, wherein the La group is: 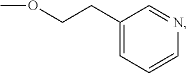
said phenyl being further substituted with one R₆ group or one Lb group, wherein the R₆ group represents cyano, halogen, or —S(C₁-C₄)alkyl, and wherein the Lb group is selected from T-(CH₂)r-D, in which either:
T is absent, r represents 0 or 1, and D represents hydroxy, CONH₂, CONHRc, C(O)NHORc, or C(O)NHNH₂, wherein Rc represents optionally substituted (C₁-C₃)alkyl or cyclopropyl; or
T is present and represents —CONH—, r represents 1 or 2, and D represents COORc or pyridyl, wherein Rc represents optionally substituted (C₁-C₃)alkyl; or
phenyl substituted with one La group, wherein the La group is 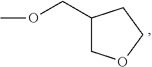
then the phenyl is further substituted one Lb group and the Lb group represents —(CH₂)r-D, wherein D represents CONH₂ or CONHRc
wherein Rc represents optionally substituted (C₁-C₃)alkyl; or
phenyl or pyridyl substituted with one La group only, wherein the La group is selected from one of the following groups: 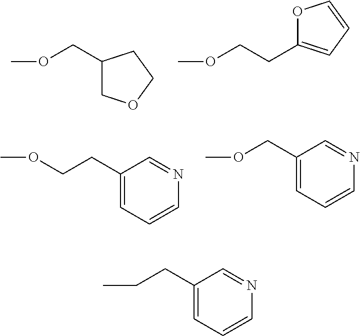
provided that
when La is 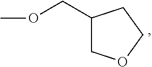
then R₇ is methyl, p represents 1, and R₄ is methyl;
when La is 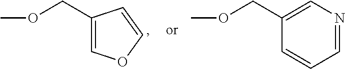
then R₇ is methyl, p represents 1, and R₄ is a cyano group which is attached at the 4-position of the phenyl A ring relative to group W; and
when La is 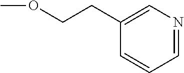
then R₇ represents unsubstituted (C₁-C₃)alkyl, and either p represents 0, or p represents I and R₄ is halogen, cyano or methyl.

8. A compound as claimed in claim 2, wherein
B is selected from one of the following:
phenyl substituted with one La group, said phenyl being further substituted with one R₆ group or one Lb group, wherein the Lb group is selected from T-(CH₂)r-D, wherein T is optionally present and independently represents —NHCO— or —CONH—; D represents hydroxy, CONH₂, CONHRc, CONRdRe, COOH, COORc, C(O)NHORc, C(O)NHNH₂, or pyridyl, wherein Rc represents optionally substituted (C₁-C₃)alkyl or cyclopropyl, and Rd and Re together form a —(CH₂)₄— group or a —(CH₂)₂—O—(CH₂)₂— group;
phenyl or pyridyl substituted with one La group only, wherein the La group is selected from one of the following groups: 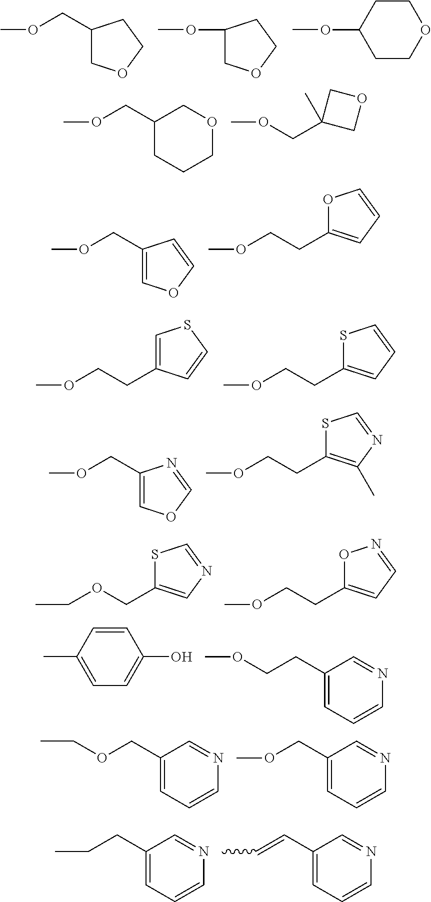
provided that
when La is 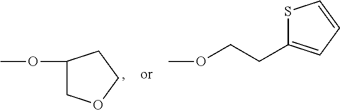
then p represents 1 and R₄ is a cyano group which is attached at the 4-position of the phenyl or pyridyl A ring relative to group W;
when La is 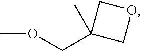
then p represents I and either R₄ is a halogen or R₄ is a cyano group which is attached at the 4-position of the phenyl or pyridyl A ring relative to group W;
when La is 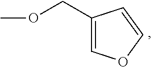
then either R₇ is methyl, p represents 1 and R₄ is a cyano group which is attached at the 4-position of the phenyl or pyridyl A ring relative to group W; or R₇ is ethyl, p represents 1 and R₄ is a cyano group; and
when La is 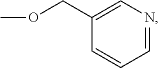
then R₇ represents unsubstituted (C₁-C₃)alkyl, and either p represents 0, or p represents 1 and R₄ is cyano, CONH₂, or methyl.

9. A compound as claimed in claim 1, said compound being selected from
N-(3-((2,4-difluorobenzyl)(4-(3-((tetrahydrofuran-3-yl)methoxy)phenoxy)benzyl)amino)-2-methylphenyl)methanesulfonamide;
N-(3-((2-cyanobenzyl)(4-(3-(2-(thiophen-3-yl)ethoxy)phenoxy)benzyl)amino)-2-methylphenyl)methanesulfonamide;
N-(2-methyl-3-((4-(3-((tetrahydrofuran-3-yl)methoxy)phenoxy)benzyl)(4-(trifluoromethyl)benzyl)amino)phenyl)methanesulfonamide;
N-(3-((3-cyanobenzyl)(4-(3-(furan-3-ylmethoxy)phenoxy)benzyl)amino)-2-methylphenyl)ethanesulfonamide;
N-(3-((3-cyanobenzyl)(4-(3-(tetrahydrofuran-3-yloxy)phenoxy)benzyl)amino)-2-methylphenyl)methanesulfonamide;
N-(3-((3-cyanobenzyl)(4-(3-((3-methyloxetan-3-yl)methoxy)phenoxy)benzyl)amino)-2-methylphenyl)methanesulfonamide;
N-(3-(((4-cyanobenzyl)(4-(3-((tetrahydrofuran-3-yl)oxy)phenoxy)benzyl)amino)-2-methylphenyl)ethanesulfonamide;
N-(3-((4-cyanobenzyl)(4-(3-((tetrahydrofuran-3-yl)oxy)phenoxy)benzyl)amino)-2-methylphenyl)methanesulfonamide;
N-(3-((3-cyanobenzyl)(4-(3-((tetrahydrofuran-3-yl)methoxy)phenoxy)benzyl)amino)-2-methylphenyl)propane-2-sulfonamide;
N-(3-((4-cyanobenzyl)(4-(3-(2-(pyridin-3-yl)ethoxy)phenoxy)benzyl)amino)-2-methylphenyl)-1,1,1-trifluoromethanesulfonamide;
N-(3-((3-cyanobenzyl)(4-(3-(2-(thiophen-3-yl)ethoxy)phenoxy)benzyl)amino)-2-methylphenyl)methanesulfonamide;
N-(3-(benzyl(4-(3-(2-(thiophen-3-yl)ethoxy)phenoxy)benzyl)amino)-2-methylphenyl)ethanesulfonamide;
N-(3-(benzyl(4-(3-(2-(thiophen-3-yl)ethoxy)phenoxy)benzyl]amino)-2-methylphenyl)methanesulfonamide hydrochloride;
N-(3-((4-cyanobenzyl)(4-(3-(2-(pyridin-3-yl)ethoxy)-5-(pyrrolidine-1-carbonyl)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-5-(2-(pyridin-3-yl)ethoxy)benzoic acid;
N-(3-((3-cyanobenzyl)(4-(3-((tetrahydrofuran-3-yl)methoxy)phenoxy)benzyl)amino)-2-methylphenyl)ethanesulfonamide;
N-(3-((3-cyanobenzyl)(4-(3-(2-(thiophen-2-yl)ethoxy)phenoxy)benzyl)amino)-2-methylphenyl)methanesulfonamide];
N-(3-((4-cyanobenzyl)(4-(3-(2-(pyridin-3-yl)ethoxy)phenoxy)benzyl)amino)phenyl)methanesulfonamide;
N-(3-((3-cyanobenzyl)(4-(3-((tetrahydrofuran-3-yl)methoxy)phenoxy)benzyl)amino)-2-methylphenyl)propane-1-sulfonamide;
N-(3-((4-cyanobenzyl)(4-(3-(furan-3-ylmethoxy)phenoxy)benzyl)amino)-2-methylphenyl)ethanesulfonamide;
3-(4-(((4-cyanobenzyl)(2-methyl-3-(methylsulfonamido)phenyl)amino)methyl)phenoxy)-N-(2-hydroxyethyl)-5-((tetrahydrofuran-3-yl)methoxy)benzamide;
N-(3-((4-cyanobenzyl)(4-(3-((tetrahydro-2H-pyran-4-yl)oxy)phenoxy)benzyl)amino)-2-methylphenyl)methanesulfonamide;
N-(3-((4-cyanobenzyl)(4-(3-(2-(4-methylthiazol-5-yl)ethoxy)phenoxy)benzyl)amino)-2-methylphenyl)methanesulfonamide;
N-(3-((4-cyanobenzyl)(4-(3-((3-methyloxetan-3-yl)methoxy)phenoxy)benzyl)amino)-2-methylphenyl)methanesulfonamide;
N-(3-((3-cyanobenzyl)(4-(3-(furan-3-ylmethoxy)phenoxy)benzyl)amino)-2-methylphenyl)methanesulfonamide;
N-(3-(benzyl(4-(3-(furan-3-ylmethoxy)phenoxy)benzyl)amino)-2-methylphenyl)ethanesulfonamide;
N-(3-((4-cyanobenzyl)(4-(3-(2-(thiophen-3-yl)ethoxy)phenoxy)benzyl)amino)-2-methylphenyl)methanesulfonamide;
N-(3-((3-fluorobenzyl)(4-(3-(furan-3-ylmethoxy)phenoxy)benzyl)amino)-2-methylphenyl)methanesulfonamide;
N-(3-((3,5-difluorobenzyl)(4-(3-((tetrahydrofuran-3-yl)methoxy)phenoxy)benzyl)amino)-2-methylphenyl)methanesulfonamide;
N-(3-((4-cyanobenzyl)(4-(3-(morpholine-4-carbonyl)-5-(2-(pyridin-3-yl)ethoxy)phenoxy)benzyl)amino)-2-methylphenyl)methanesulfonamide;
N-(3-((3-fluorobenzyl)(4-(3-((3-methyloxetan-3-yl)methoxy)phenoxy)benzyl)amino)-2-methylphenyl)methanesulfonamide;
N-(3-((4-cyanobenzyl)(4-(3-((tetrahydrofuran-3-yl)methoxy)phenoxy)benzyl)amino)-2-methylphenyl)ethanesulfonamide;
N-(3-((4-cyanobenzyl)(4-(3-(furan-3-ylmethoxy)phenoxy)benzyl)amino)-2-methylphenyl)methanesulfonamide;
N-(3-((3-fluorobenzyl)(4-(3-((tetrahydrofuran-3-yl)methoxy)phenoxy)benzyl)amino)-2-methylphenyl)ethanesulfonamide;
N-(3-((4-cyanobenzyl)(4-(3-(2-(thiophen-2-yl)ethoxy)phenoxy)benzyl)amino)-2-methylphenyl)methanesulfonamide;
2-(3-(4-(((4-cyanobenzyl)(2-methyl-3-(methylsulfonamido)phenyl)amino)methyl)phenoxy)-5-(2-(pyridin-3-yl)ethoxy)benzamido)acetic acid;
N-(3-((4-chloro-3-fluorobenzyl)(4-(3-((tetrahydrofuran-3-yl)methoxy)phenoxy)benzyl)amino)-2-methylphenyl)methanesulfonamide;
4-(((2-methyl-3-(methylsulfonamido)phenyl)(4-(3-(2-(pyridin-3-yl)ethoxy)phenoxy)benzyl)amino)methyl)benzamide;
methyl 3-(4-(((4-cyanobenzyl)(2-methyl-3-(methylsulfonamido)phenyl)amino)methyl)phenoxy)-5-(2-(pyridin-3-yl)ethoxy)benzoate;
3-(4-(((4-cyanobenzyl)(2-methyl-3-(methylsulfonamido)phenyl)amino)methyl)phenoxy)-N-methyl-5-(pyridin-3-ylmethoxy)benzamide;
N-(2-methyl-3-((3-methylbenzyl)(4-(3-((tetrahydrofuran-3-yl)methoxy)phenoxy)benzyl)amino)phenyl)methanesulfonamide;
ethyl 2-(3-(4-(((4-cyanobenzyl)(2-methyl-3-(methylsulfonamido)phenyl)amino)methyl)phenoxy)-5-((tetrahydrofuran-3-yl)methoxy)benzamido)acetate;
3-(4-(((4-cyanobenzyl)(2-methyl-3-(methylsulfonamido)phenyl)amino)methyl)phenoxy)-N-(pyridin-2-ylmethyl)-5-((tetrahydrofuran-3-yl)methoxy)benzamide;
N-(3-((3-fluorobenzyl)(4-(3-((tetrahydrofuran-3-yl)methoxy)phenoxy)benzyl)amino)-2-methylphenyl)methanesulfonamide;
N-(3-((3-chlorobenzyl)(4-(3-((tetrahydrofuran-3-yl)methoxy)phenoxy)benzyl)amino)-2-methylphenyl)methanesulfonamide;
N-(3-((3-cyanobenzyl)(4-(3-(pyridin-3-ylmethoxy)phenoxy)benzyl)amino)-2-methylphenyl)ethanesulfonamide;
N-(3-((4-chlorobenzyl)(4-(3-((tetrahydrofuran-3-yl)methoxy)phenoxy)benzyl)amino)-2-methylphenyl)methanesulfonamide;
N-(3-((3-fluorobenzyl)(4-(3-(pyridin-3-ylmethoxy)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-N,N-dimethyl-5-(2-(pyridin-3-yl)ethoxy)benzamide;
3-(4-(((4-cyanobenzyl)(2-methyl-3-(methylsulfonamido)phenyl)amino)methyl)phenoxy)-5-(pyridin-3-ylmethoxy)benzamide;
3-(4-(((4-cyanobenzyl)(2-methyl-3-(methylsulfonamido)phenyl)amino)methyl)phenoxy)-N-methyl-5-((tetrahydrofuran-3-yl)methoxy)benzamide;
N-(2-methyl-3-((4-methylbenzyl)(4-(3-((tetrahydrofuran-3-yl)methoxy)phenoxy)benzyl)amino)phenyl)methanesulfonamide;
N-(3-(((4-cyanobenzyl)(4-(3-(pyridin-3-ylmethoxy)phenoxy)benzyl)amino)-2-methylphenyl)ethanesulfonamide;
N-(3-((3-cyanobenzyl)(4-(3-(pyridin-3-ylmethoxy)phenoxy)benzyl)amino)-2-methylphenyl)methanesulfonamide;
N-(3-((3-cyanobenzyl)(4-(3-((tetrahydrofuran-3-yl)methoxy)phenoxy)benzyl)amino)-2-methylphenyl)methanesulfonamide;
N-(3-((3-fluorobenzyl)(4-(3-(2-(pyridin-3-yl)ethoxy)phenoxy)benzyl)amino)-2-methylphenyl)methanesulfonamide;
N-(3-((4-cyanobenzyl)(4-(4-fluoro-3-(2-(pyridin-3-yl)ethoxy)phenoxy)benzyl)amino)-2-methylphenyl)methanesulfonamide;
N-(3-((4-cyanobenzyl)(4-(3-((tetrahydrofuran-3-yl)methoxy)phenoxy)benzyl)amino)-2-methylphenyl)methanesulfonamide;
N-(3-((4-cyanobenzyl)(4-(3-(pyridin-3-ylmethoxy)phenoxy)benzyl)amino)-2-methyl phenyl)methanesulfonamide;
N-(3-((3-cyanobenzyl)(4-(3-(2-(pyridin-3-yl)ethoxy)phenoxy)benzyl)amino)-2-methyl phenyl)ethanesulfonamide;
3-(4-(((4-cyanobenzyl)(2-methyl-3-(methylsulfonamido)phenyl)amino)methyl)phenoxy)-5-((tetrahydrofuran-3-yl)methoxy)benzamide;
N-(3-((3-cyanobenzyl)(4-(3-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)-3-fluorophenoxy)-5-((tetrahydrofuran-3-yl)methoxy)benzamide;
N-(3-((4-cyanobenzyl)(4-(3-(2-(pyridin-3-yl)ethoxy)phenoxy)benzyl)amino)-2-methylphenyl)ethanesulfonamide;
3-(4-(((4-cyanobenzyl)(2-methyl-3-(methylsulfonamido)phenyl)amino)methyl)phenoxy)-N-cyclopropyl-5-(2-(pyridin-3-yl)ethoxy)benzamide;
3-(4-(((4-cyanobenzyl)(2-methyl-3-(methylsulfonamido)phenyl)amino)methyl)phenoxy)-N-(pyridin-2-ylmethyl)-5-(2-(pyridin-3-yl)ethoxy)benzamide;
3-(4-(((4-cyanobenzyl)(2-methyl-3-(methylsulfonamido)phenyl)amino)methyl)phenoxy)-5-(2-(pyridin-3-yl)ethoxy)-N-(pyridin-4-ylmethyl)benzamide;
3-(4-(((4-cyanobenzyl)(2-methyl-3-(methylsulfonamido)phenyl)amino)methyl)phenoxy)-5-(2-(pyridin-3-yl)ethoxy)-N-(pyridin-3-ylmethyl)benzamide;
N-(3-((4-cyanobenzyl)(4-(3-(2-(pyridin-3-yl)ethoxy)phenoxy)benzyl)amino)-2-methylphenyl)methanesulfonamide;
ethyl 2-(3-(4-(((4-cyanobenzyl)(2-methyl-3-(methylsulfonamido)phenyl)amino)methyl)phenoxy)-5-(2-(pyridin-3-yl)ethoxy)benzamido)acetate;
3-(4-(((4-cyanobenzyl)(2-methyl-3-(methylsulfonamido)phenyl)amino)methyl)phenoxy)-N-ethyl-5-(2-(pyridin-3-yl)ethoxy)benzamide;
N-(3-((4-cyanobenzyl)(4-(3-(hydrazinecarbonyl)-5-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)phenoxy)-N-methoxy-5-(2-(pyridin-3-yl)ethoxy)benzamide;
3-(4-(((4-cyanobenzyl)(2-methyl-3-(methylsulfonamido)phenyl)amino)methyl)phenoxy)-N-methyl-5-(2-(pyridin-3-yl)ethoxy)benzamide;
3-(4-(((4-cyanobenzyl)(2-methyl-3-(methylsulfonamido)phenyl)amino)methyl)phenoxy)-5-(2-(pyridin-3-yl)ethoxy)benzamide;
N-(3-((4-cyanobenzyl)(4-(3-(hydroxymethyl)-5-(2-(pyridin-3-yl)ethoxy)phenoxy)benzyl)amino)-2-methylphenyl)methanesulfonamide;
3-(4-(((4-cyanobenzyl)(2-methyl-3-(methylsulfonamido)phenyl)amino)methyl)-2-fluorophenoxy)-5-((tetrahydrofuran-3-yl)methoxy)benzamide;
N-(3-(benzyl(4-(3-(2-(pyridin-3-yl)ethoxy)phenoxy)benzyl)amino)-2-methylphenyl)methanesulfonamide;
N-(3-((4-cyanobenzyl)(4-(3-(hydroxymethyl)-5-(pyridin-3-ylmethoxy)phenoxy)benzyl)amino)-2-methylphenyl)methanesulfonamide;
N-(3-((3-cyanobenzyl)(4-(3-(hydroxymethyl)-5-((tetrahydrofuran-3-yl)methoxy)phenoxy)benzyl)amino)-2-methylphenyl)methanesulfonamide;
N-(3-((4-cyanobenzyl)(4-(3-(hydroxymethyl)-5-((tetrahydrofuran-3-yl)methoxy)phenoxy)benzyl)amino)-2-methylphenyl)methanesulfonamide;
N-(3-(((4-(3-cyano-5-((tetrahydrofuran-3-yl)methoxy)phenoxy)benzyl)(4-cyanobenzyl)amino)-2-methylphenyl)methanesulfonamide;
3-(4-(((4-cyano-3-fluorobenzyl)(2-methyl-3-(methylsulfonamido)phenyl)amino)methyl)phenoxy)-5-(2-(pyridin-3-yl)ethoxy)benzamide;
N-(3-((4-(3-cyano-5-(2-(pyridin-3-yl)ethoxy)phenoxy)benzyl)(4-cyanobenzyl)amino)-2-methylphenyl)methanesulfonamide;
N-(3-((4-cyanobenzyl)(4-(3-(hydroxymethyl)-5-(pyridin-3-yl)methoxy)phenoxy)benzyl)amino)-2-methylphenyl)ethanesulfonamide;
N-(3-((3-cyanobenzyl)(4-(3-(hydroxymethyl)-5-((tetrahydrofuran-3-yl)methoxy)phenoxy)benzyl)amino)-2-methylphenyl)ethanesulfonamide;
N-(3-(benzyl(4-(3-((tetrahydro-2H-pyran-3-yl)methoxy)phenoxy)benzyl)amino)-2-methylphenyl)methanesulfonamide;
N-(3-(benzyl(4-(3-(3-(methylthio)propoxy)phenoxy)benzyl)amino)-2-methylphenyl)methanesulfonamide;
N-(3-(benzyl(4-(3-(pyridin-3-yloxy)phenoxy)benzyl)amino)-2-methylphenyl)methanesulfonamide;
N-(3-(benzyl(4-(3-(2-(pyridin-4-yl)ethoxy)phenoxy)benzyl)amino)-2-methylphenyl)methanesulfonamide;
N-(3-((4-(3-(2-(1H-pyrazol-1-yl)ethoxy)phenoxy)benzyl)(benzyl)amino)-2-methylphenyl)methanesulfonamide;
N-(3-(benzyl(4-(3-(2-(isoxazol-5-yl)ethoxy)phenoxy)benzyl)amino)-2-methylphenyl)methanesulfonamide;
N-(3-(benzyl(4-(3-(2-(furan-2-yl)ethoxy)phenoxy)benzyl)amino)-2-methylphenyl)methanesulfonamide;
N-(3-(benzyl(4-(3-(thiazol-5-ylmethoxy)phenoxy)benzyl)amino)-2-methylphenyl)methanesulfonamide;
N-(3-(benzyl(4-(3-(oxazol-4-ylmethoxy)phenoxy)benzyl)amino)-2-methylphenyl)methanesulfonamide;
N1-(4-(4-(((4-cyanobenzyl)(2-methyl-3-(methylsulfonamido)phenyl)amino)methyl)phenoxy)-2-(2-(pyridin-3-yl)ethoxy)phenyl)-N3-ethylmalonamide;
N1-(4-(4-(((4-cyanobenzyl)(2-methyl-3-(methylsulfonamido)phenyl)amino)methyl)phenoxy)-2-(2-(pyridin-3-yl)ethoxy)phenyl)-N3-methylmalonamide;
N-(3-((4-cyanobenzyl)(4-(4′-hydroxybiphenyl-3-yloxy)benzyl)amino)-2-methylphenyl)methanesulfonamide;
3-(3-(4-(((4-cyanobenzyl)(2-methyl-3-(methylsulfonamido)phenyl)amino)methyl)phenoxy)-5-(2-(pyridin-3-yl)ethoxy)phenylamino)-3-oxopropanoic acid;
N-(3-((4-cyanobenzyl)(4-(3-(2-(furan-2-yl)ethoxy)phenoxy)benzyl)amino)-2-methylphenyl)methanesulfonamide;
N-(3-((4-cyanobenzyl)(4-(2-(2-(pyridin-3-yl)ethoxy)pyridin-4-yloxy)benzyl)amino)-2-methylphenyl)methanesulfonamide;
N-(3-((3-cyanobenzyl)(4-(3-(2-(furan-2-yl)ethoxy)phenoxy)benzyl)amino)-2-methylphenyl)methanesulfonamide;
N-(3-((4-cyanobenzyl)(4-(5-(2-(pyridin-3-yl)ethoxy)pyridin-3-yloxy)benzyl)amino)-2-methylphenyl)methanesulfonamide;
(E)-N-(3-((4-cyanobenzyl)(4-(3-(2-(pyridin-3-yl)vinyl)phenoxy)benzyl)amino)-2-methylphenyl)methanesulfonamide;
N-(3-((4-cyanobenzyl)(4-(3-(2-(pyridin-3-yl)ethyl)phenoxy)benzyl)amino)-2-methylphenyl)methanesulfonamide;
N-(3-((4-cyanobenzyl)(4-(3-((pyridin-3-ylmethoxy)methyl)phenoxy)benzyl)amino)-2-methylphenyl)methanesulfonamide;
N-(3-(((4-cyanobenzyl)(4-(3-((thiazol-5-ylmethoxy)methyl)phenoxy)benzyl)amino)-2-methylphenyl)methanesulfonamide;
N-(3-((4-cyanobenzyl)(4-(2-fluoro-6-(2-(pyridin-3-yl)ethoxy)pyridin-4-yloxy)benzyl)amino)-2-methylphenyl)methanesulfonamide;
N-(3-((4-cyanobenzyl)(4-(2-(propylthio)-6-(2-(pyridin-3-yl)ethoxy)pyridin-4-yloxy)benzyl)amino)-2-methylphenyl)methanesulfonamide;
N-(3-((4-(3-cyano-5-((tetrahydrofuran-3-yl)methoxy)phenoxy)-3-fluorobenzyl)(4-cyanobenzyl)amino)-2-methyl phenyl)methanesulfonamide;
N-(3-((4-(3-cyano-5-(pyridin-3-ylmethoxy)phenoxy)benzyl)(4-cyanobenzyl)amino)-2-methylphenyl)methanesulfonamide;
or a pharmaceutically acceptable salt thereof.

10. A pharmaceutical composition which comprises a compound as claimed in claim 1, together with a pharmaceutically acceptable carrier.

11. A pharmaceutical composition as claimed in claim 10, which further comprises one or more additional active agents.

12. (canceled)

13. (canceled)

14. (canceled)

15. A method for the treatment or prophylaxis of a disease or disorder for which glucocorticoid treatment is indicated or is likely to be effective in a mammal, which comprises administering to the mammal a therapeutically effective amount of a compound as claimed in claim 1.

16. A method as claimed in claim 15, wherein the condition associated with a disease or disorder for which glucocorticoid treatment is indicated or is likely to be effective is selected from inflammatory, immune, and allergic conditions.

17. A method as claimed in claim 15, wherein the condition associated with a disease or disorder for which glucocorticoid treatment is indicated or is likely to be effective is selected from inflammation and arthritis.

18. A pharmaceutical composition which comprises a compound as claimed in claim 9, together with a pharmaceutically acceptable carrier.
